SEQUENCE LISTING (32 sequences)

SEQ ID NO: 1 - DNA, 1977 bp, unknown
  Solanum lycopersicum ARF9 coding sequence from cv Moneymaker
atggcaactataaatgggtggtgttatgagtctcagccgaatatgaattctccaggtaaaaaagatgctctgtatcatgagctatggcagttgtgtgcaggtccagtagttgatgttcccagggaaggagaaagagtttattattttcctcaaggccacatggaacaattggtagcatcaattaatcaagaaatggatcaaagagttccatcattcaatctcaaatcaaaggtcctttgtcgagttatcaatagtcatttcctggctgaagaagacaatgatgaggtctatgtccagatcactttgatgccagaggcaccacatgtacccgagccgactactccggatccattaattccgcaggatgtaaagcctagattccattctttctgcaaggtcctgacagcctccgatacaagcactcatggtggattttctgttctaaggaaacatgctaatgaatgccttcctccattggacttgaaccagcagactccgacccaggaattgattgcgaaagaccttcatgacgtggagtggcgcttcaagcatatatttagaggccaacctcggagacatttacttaccacagggtggagtacctttgtttcttcaaagaaattagtggcaggggattcttttgtattcttgaggggtaataatggacagctgcgagttggggtcaaaaggcttgttcgccagcagagctcaatgccgtcatcggtgatgtcaagccagagcatgcacctaggagtcttggctacagcatctcatgctgttacaacccagacaatgtttgttgtttactacaaaccgagaaccactcagttcatcgtaggcgtcaacaaatacttagaggctcttaaacatgaatatgcagttggcatgcgattcaaaatgcagtttgaagccgaagggaatcctgatagaagatttatgggcactatagttggaattgatgatctttcttcacagtggaaaaattctgcgtggcgatccttgaaggtccgatgggacgagcctgcagccattgcaaggcctgacagagtttctccttgggaaattaaaccttatgtgtgttcaattccaaatgtccttgtcccaccaaccgcagagaagaacaaaaggcatcggctacatagtgaaatcaaaatatcagaacaaccttcatcctcaaatgcttcggcggtttggaatccttcccttcgatcgcctcagtttaacacctttggcatcaacagcagtactaattgcgcattagcgtctcttacagagagtggttggcagcttcctcatttaaatacttcaggtatgcttgtggatgagccagaagacggtaggagtgctccaacttggtgtggttttccatgcgttttggccccgcagttcggtcaagggactaatcagccgattgttattcctactgatgggagaaaatgtgataccaaaaaaacctgtagattatttggtattgacttgaaaagttcctcaattagcactactgaggcacgactacagctacagccagccggcatttcttgtgtctttgcagagagagcacctccaaacacggtgcctgctggtgattcagatcaaaagtctgagctttcagtagacttcaaagatcaaatgcaaggccatttgcggttacccctaaaggaggttcaaagcaagcagagttgttccaccaggtctcgcacaaaggtgcaaatgcaaggcgtagctgtaggtcgtgcagtggatttaaccatattgaaaggatacgatgagcttacaaaggagcttgaggagatgtttgaaatccaaggagagcttcagtcacgacagaaatgggggatcttgtttacagatgatgaaggggatacaatgcttatgggtgattatccgtggcaagacttttgcaatgtggtgaggaagattttcatttgttcaagtcaggatatgaaaaaattgaccctgtctcgcgcagaccattaa

SEQ ID NO: 2 - protein, 658 aa, unknown
  Solanum lycopersicum ARF9 protein from cv Moneymaker
MATINGWCYESQPNMNSPGKKDALYHELWQLCAGPVVDVPREGERVYYFPQGHMEQLVASINQEMDQRVPSFNLKSKVLCRVINSHFLAEEDNDEVYVQITLMPEAPHVPEPTTPDPLIPQDVKPRFHSFCKVLTASDTSTHGGFSVLRKHANECLPPLDLNQQTPTQELIAKDLHDVEWRFKHIFRGQPRRHLLTTGWSTFVSSKKLVAGDSFVFLRGNNGQLRVGVKRLVRQQSSMPSSVMSSQSMHLGVLATASHAVTTQTMFVVYYKPRTTQFIVGVNKYLEALKHEYAVGMRFKMQFEAEGNPDRRFMGTIVGIDDLSSQWKNSAWRSLKVRWDEPAAIARPDRVSPWEIKPYVCSIPNVLVPPTAEKNKRHRLHSEIKISEQPSSSNASAVWNPSLRSPQFNTFGINSSTNCALASLTESGWQLPHLNTSGMLVDEPEDGRSAPTWCGFPCVLAPQFGQGTNQPIVIPTDGRKCDTKKTCRLFGIDLKSSSISTTEARLQLQPAGISCVFAERAPPNTVPAGDSDQKSELSVDFKDQMQGHLRLPLKEVQSKQSCSTRSRTKVQMQGVAVGRAVDLTILKGYDELTKELEEMFEIQGELQSRQKWGILFTDDEGDTMLMGDYPWQDFCNVVRKIFICSSQDMKKLTLSRADH

SEQ ID NO: 3 - DNA, 7510 bp, unknown
  ARF9 promoter and gene from Solanum lycopersicum cv Moneymaker
acagagtcactcgtaatgtaaattttttcatatctaatatttaatttaatgttctcgatttagagtaaagaattctcaacatgataattagtactttttaattttcgcacgcttttactttttaatatgatagcggagtcacttatgacgtaaatttctccacatctaagactacaaatctaatgtcttcgattgagacttaagaattctcaccgttacatattaaattataatcttgacttttatgcaatttttacggtagttattccaccattacacgacaggcggtgttaactttacttagttaataaccagcccgctacttacccaaccaacgccccacacacttaaatccaaggtccccaattaagactacagaaattttgccgttacataataattaattctcttgactttcacacttttttacttcttaataatataacagagtcacccgtaacgtaaatttttccatatctaagatttaaatctaatgtcctcgattaagagcaaaaaactcccaccattacatgataattagtactttttgactttcgcacgctttcactttttaatacaataacgggatcactcctaagtcgtaacataaatgtcaccgcatctaacacttaaatctaatatcctctattaagactaaagaaatccaaccattacatgataattagtacttctcgagtttcgcacgcttttacttcttaacataataactgagtcatttgtaacgtaaatttctccacatctaaaactcacatccaatatctttgattaagacttaagaattctcaccgttacataataattaataatcttgacttttatgcaatttttacggtagttgttccaccattgcttgacaggtggtgttaactttacttagttaataaccacccgttacttacccaaccaacccccccccccgcacacacacttaaatccaaggtccccgattaaagactaaagaaatctcgccgttacataataattaatactcttgactttcacacctttttacttcttaatacaataacactcgtaacgtaaatttttccatatctaagatttaaatctaatgtcctcgattaagagtaaaatctcttaccattacatgataattagtactttttgactttcgcacgcttttactttttaatacaataacggggtcacttccaattagtcgtaacgtaaatatcaccgcatctaagacataaatccaatgtactcgattaagactaaaaaaatccaaccattacatgataattaacacttctggactttcgcacgcttttacttcttaacacaaataatggagtcacttgtaaaccataaatttctccacctttaacactcaaatctactatctccgattaggactaaaaaacaaaaaactccacggttacatactaattaatatgtttgacttttaaccacaattttttacagtagttattccgcccaatacacgaaagcggggtttactctacttttaaccaagcttataaaacccaaccaaccccccactttacttcacactacacagatgaaaaaaaggagagaaacacgttctctgcagggagcggtagaaagcaacaaatcaagccatatattaagctgtttttgggtaaactccgttacagagttttctctttctctctctcactttctctctttctctcacgtacaaaatagaaagaaaaaaaaaatgaaattattaagctgttgcttcttctgttgttgctcctgtttgttccagtagaactagtactactattgttagttaatttttttttatattcagcactactttaaactttatttttagttgttgggggtgtttctttaaggcttttttatgtgttgttgttatagcagggaagaaatgtggtgaattttagctagttttgtgatgtttttttgagctaattacttgattttgaattctgggtgttctttagtttttggaattgaaggttgattatactcatggcaactataaatgggtggtgttatgagtctcagccgaatatgaattctccaggtgaaagtttgattttttgaagtttaatttgagaattttggagctttttttgttttttttgtttaaaatgtggattttttattttttgagtttcaggtaaaaaagatgctctgtatcatgagctatggcagttgtgtgcaggtccagtagttgatgttcccagggaaggagaaagagtttattattttcctcaaggccacatggaacaagtaaaaaattttattttaaaaaaataattgaattctgtgtttttttttctttctgttttaggtgaatctggttgattcaaggctaagttcttgtttttgagttatggagttgtgaatttttattgaatttctcaatttgagtgtatatttttagttatgttttctggggattgtaaaatctgtatctttctctttttgtctcactttttttggttttgtggaaaagttggtagcatcaattaatcaagaaatggatcaaagagttccatcattcaatctcaaatcaaaggtcctttgtcgagttatcaatagtcatttcctggtatgtttatatttcatggtgttatttgatcagtttattttacatacattgtattcatacttgttagtaattcatccaaaaaaagtgatgaatttattgttgggttcaaaattcaaacatcggaaggtgagccttggcgtattcgtagctggcaaagttgtcggtttgtcgccaagtgaacgaccatgaggtcaccgttcaatgcgtgtaagcaatctcttgcagaaatgcagggtaaggttgtgtccaacagacccttatagtccagtctagcgggagctttagtgcacctgaatgcgtgtaaagttcatacaattttgtatttgaaatgtaaaatgtttttgatgattgcttgtaggctgaagaagacaatgatgaggtctatgtccagatcactttgatgccagaggcaccacatgtaagaagttaaaattgttgtagtattcacttgtttatatgcattttattgatcaatttggttttaattattgatctgatttttaggtacccgagccgactactccggatccattaattccgcaggatgtaaagcctagattccattctttctgcaaggtcctgacagcctccgatacaagcactcatggtggattttctgttctaaggaaacatgctaatgaatgccttcctccattggtaatataagtgtgatcattttgtataggctagactacgtttccccccctgttttgacaagttgttgaattagctgtcgtgtttgttatactttgttaggacttgaaccagcagactccgacccaggaattgattgcgaaagaccttcatgacgtggagtggcgcttcaagcatatatttagaggtaattgttttgtttgtagagtgtaacagaagatatcaatgaagctttatcatgtattgtaaaggtcaaaagtcatccgtctttgttttaattataacaggccaacctcggagacatttacttaccacagggtggagtacctttgtttcttcaaagaaattagtggcaggggattcttttgtattcttgaggtatctctctctggtcctagcttctcgtatgagtatgatatttgattgcattgtctatcactattgcatgtcatgtcatatatccagaacagttaatgttggtttgttaataatcatattctaatggatatgtacgctctaggggtaataatggacagctgcgagttggggtcaaaaggcttgttcgccagcagagctcaatgccgtcatcggtgatgtcaagccagagcatgcacctaggagtcttggctacagcatctcatgctgttacaacccagacaatgtttgttgtttactacaaaccgaggtttgtcacatacacctcctttcaattatcccgtcatccttacgcagtagagatcattttcatgccagagaacatactaaatctttcatatgctcttccaattttctgctttgacaaccttcagaaccactcagttcatcgtaggcgtcaacaaatacttagaggctcttaaacatgaatatgcagttggcatgcgattcaaaatgcagtttgaagccgaagggaatcctgatagaaggtagtgatatcaattactcgctagctccattttgatttatctgtcttactttcctttatagtttgtttaaaagaatgcatctttccctttttggctttaattcaacttaaatcccgtgccaagttaaaaccagcaaataaattgaaatggtgggagtattcagaagtcctccaattagatgtggaagtctttgatcaagatgtttatttgccaactgcttagttctgacatgtatttgtttgggctctcccctattatttctcagatttatgggcactatagttggaattgatgatctttcttcacagtggaaaaattctgcgtggcgatccttgaaggttgatatctacatttatatttcagcaatattctttaagttcaattggaaatcaccaattctcccttcgttccaggtccgatgggacgagcctgcagccattgcaaggcctgacagagtttctccttgggaaattaaaccttatgtgtgttcaattccaaatgtccttgtcccaccaaccgcagagaagaacaaaaggcatcggctacatagtgaaatcaaaatatcaggtctgaatgaaaattctcaacagcatggtggtttttcatgtgttggttgctgatattttaatttccattttcattttcttaattatcttgcctttcttttcctgttagaacaaccttcatcctcaaatgcttcggcggtttggaatccttcccttcgatcgcctcagtttaacacctttggcatcaacagcagtactaattgcgcattagcgtctcttacagagagtggttggcagcttcctcatttaaatacttcaggtatgcttgtggatgagccagaagacggtaggagtgctccaacttggtgtggttttccatgcgttttggccccgcagttcggtcaagggactaatcagccgattgttattcctactgatgggagaaaatgtgataccaaaaaaacctgtagattatttggtattgacttgaaaagttcctcaattagcactactgaggcacgactacagctacagccagccggcatttcttgtgtctttgcagagagagcacctccaaacacggtgcctgctggtgattcagatcaaaagtctgagctttcagtagacttcaaagatcaaatgcaaggccatttgcggttacccctaaaggaggttcaaagcaagcagagttgttccaccaggtctcgcacaaaggtattaaatcaaaagccaaaaaattgcattcttaccaactcattttcgccacaaaacatattattaaactctctaacatcttttcacatgtggaatcccttgagaaggtgcaaatgcaaggcgtagctgtaggtcgtgcagtggatttaaccatattgaaaggatacgatgagcttacaaaggagcttgaggagatgtttgaaatccaaggagagcttcagtcacgacagaaatgggggatcttgtttacagatgatgaaggggatacaatgcttatgggtgattatccgtggcagtaagttctctctcacttaaaaaaatttaagactgaatacatatacaaccactgcataagatcttttctacaacctgtaatttaacacatcacttaaatcacttaattgcagagacttttgcaatgtggtgaggaagattttcatttgttcaagtcaggatatgaaaaaattgaccctgtctcgcgcagaccattaagcagctcctttcaaatgaaatgatggcggaagatggtgaaagttaaagactccgccttttgaagtggtaccgttttgtgatctctttggcttcattctctatatatagtaggaaagaacgacaggttgttgacatagcaaggcgtagaagcttgtttttacgctctttttagcctagtgcagctccgttttgagagcagttaagtaagtaactagttataataggataagactagtaaaaatatagtactagaagtagtaaaaagttaagggtgttctctaatggggagtctatatagtagtagtttatgtagttttctattcgcgcgttaacctgtaaatactcatgtgccattgtagtattctggaaatactacaatgtatggtcttgttaacgtgctttatgccttatcttatatgaaaactccatggtggagattgctctgtacttgctctttctgtatgacttttgtgatgcacaatagggatttcttgtctaaatttagactcttttggcacaatcatgcatcactttgtaactttcccctttactgaacaaaaaaatctttcaaaacgctccttcgcgtgtgtgtttgttttattctttgctaaagggtgatcctaacatttaaattagataccgttttctttactataagggtgacttgaactcaaaacctgttttattagtatgttactctatttagaagtttaataatttattagatatgacatttctatttaccttgctatttcttttaacaatatttacctttagatttatataagtttttgattcattaactgactttggttaagatgttgagtgcaaaataaatagatggttcatcttttgagctagcgtagagtcattttcctttaaactttataaagcacttacaatcacatatttgtgcattcttagtgcacgtttgaccatggataattttcactattttatggaactgtatattataatttttttggaattagaagaactcaaaaagactcttttcataattttattccgaatcactcgtacaaaaatcaaaaaacaatttcaagttgtattcattttcaagcaaaagttcaatttgtcactatcacttgtaacacaaatattttccctttcgaatttcacaattttaatattctaagttcctttttgagaacttttagtgaaaggaaaattgggctttgtgaattgtttggtcttcaatatccctatcctagctaggaagagtgtccatatagtcatgggcctcatgagttgtcaaatgttacgtaaaggtatttatctttgcatgagtgtttggtcacgctcaaaacttctttattataagtctgagtctttgtgttatgtatccatgcttggcttgcatgatgtttgtgtcattcctaatcttattgaatggactataaaggacttagtcaagtataactataaaatgacgatagcttatagacaacttcgtagttgttggagtatacaacacgctgacatcgacttcttggagtttagtaatcataattttttttaaaaaaaacaaatctcctcaatcaacttcttgattttagtagtaatccatcgattcaggtacattcatgacgtgatgtgttcgattaatattt

SEQ ID NO: 4 - DNA, 7500 bp, unknown
  ARF9 promoter and gene from Solanum lycopersicum cv Heinz 1706
acagagtcactcgtaatgtaaattttttcatatctaatatttaatttaatgttctcgatttagagtaaagaattctcaacatgataattagtactttttaattttcgcacgcttttactttttaatatgatagcggagtcacttatgacgtaaatttctccacatctaagactacaaatctaatgtcttcgattaagacttaagaattctcaccgttacatattaaattataatcttgacttttatgcaatttttacggtagttattccaccattacacgacaggcggtgttaactttacttagttaataaccagcccgttacttacccaaccaacgccccacacacttaaatccaaggtccccaattaagactacagaaattttgccgttacataataattaattctcttgactttcacacttttttacttcttaataatataacagagtcacccgtaacgtaaatttttccatatctaagatttaaatctaatgtcctcgattaagagcaaaaaactcccaccattacatgataattagtactttttgactttcgcacgctttcactttttaatacaataacgggatcactcctaagtcgtaacataaatgtcaccgcatctaacacttaaatctaatatcctctattaagactaaagaaatccaaccattacatgataattagtacttctcgagtttcgcacgcttttacttcttaacataataactgagtcatttgtaacgtaaatttctccacatctaaaactcacatccaatatctttgattaagacttaagaattctcaccgttacataataattaataatcttgacttttatgcaatttttacggtagttgttccaccattgcttgacaggtggtgttaactttacttagttaataaccacccgttacttacccaaccaaccccccccccccccccgcacacacacttaaatccaaggtccccgattaaagactaaagaaatctcgccgttacataataattaatactcttgactttcacacctttttacttcttaatacaataacactcgtaacgtaaatttttccatatctaagatttaaatctaatgtcctcgattaagagtaaaatctcttaccattacatgataattagtactttttgactttcgcacgcttttactttttaatacaataacggggtcacttccaattagtcgtaacgtaaatatcaccgcatctaagacataaatccaatgtactcgattaagactaaaaaaatccaaccattacatgataattaacacttctggactttcgcacgcttttacttcttaacacaataatggagtcacttgtaacataaatttctccacctttaacactcaaatctactatctccgattaggactaaaacaaaactcacggttacatactaattaatatgtttgacttttacacaatttttacagtagttattccgccaatacacgaaagcggtgttaactctacttctaaccaagcttataaaacccaaccaaccccccactctacttcacactacacagatgaaaaaaggagagaaacacgttctctgcagggagcggtagaaagcaacaaatcaagccatatattaagctgtttttgggtaaactccgttacagagttttctctttctctctctcactttctctctttctctcacatacaaaatagaaagaaaaaaaaaatgaaattattaagctgttgcttcttctgttgttgctcctgtttgttccagtagaactagtactactattgttagttaattttttttatattcagcactactttaaactttatttttagttgttgggggtgtttctttaaggcttttttatgtgttgttgttatagcagggaagaaatgtggtgaattttagctagttttgtgatgtttttttgagctaattacttgattttgaattctgggtgttctttagtttttggaattgaaggttgattatactcatggcaactataaatgggtggtgttatgagtctcagccgaatatgaattctccaggtgaaagtttgattttttgaagtttaatttgagaattttggagctttttttgtttttttgtttaaaatgtggattttttattttttgagtttcaggtaaaaaagatgctctgtatcatgagctatggcagttgtgtgcaggtccagtagttgatgttcccagggaaggagaaagagtttattattttcctcaaggccacatggaacaagtaaaaaattttattttaaaaaaataattgaattctgtgttgttgtttttttctgttttaggtgaatctggttgattcaaggctaagttcttgtttttgagttatggagttgtgaatttttattgaatttctcaatttgagtgtatatttttagttatgttttctggggattgtaaaatctgtatctttctctttttgtctcactttttttggttttgtggaaaagttggtagcatcaattaatcaagaaatggatcaaagagttccatcattcaatctcaaatcaaaggtcctttgtcgagttatcaatagtcatttcctggtatgtttatatttcattgtgttatttgatcagtttattttacatacattgtattcatacttgttagtaattcatccaaaaaaagtgatgaatttattgttgggttcaaaattcaaacatcggaaggtgagccttggcgtattcgtagctggcaaagttgtcggtttgtcgccaagtgaacgaccatgaggtcaccgttcaatgcgtgtaagcaatctcttgcagaaatgcagggtaaggttgtgtccaacagacccttatagtccagtctagcgggagctttagtgcacctgaatgcgtgtaaagttcatacaattttgtatttgaaatgtaaaatgtttttgatgattgcttgtaggctgaagaagacaatgatgaggtctatgtccagatcactttgatgccagaggcaccacatgtaagaagttaaaattgttgtagtattcacttgtttatatgcattttattgatcaatttggttttaattattgatctgatttttaggtacccgagccgactactccggatccattaattccgcaggatgtaaagcctagattccattctttctgcaaggtcctgacagcctccgatacaagcactcatggtggattttctgttctaaggaaacatgctaatgaatgccttcctccattggtaatataagtgtgatcattttgtataggctagactacgtttccccccctgttttgacaagttgttgaattagctgtcgtgtttgttatactttgttaggacttgaaccagcagactccgacccaggaattgattgcgaaagaccttcatgacgtggagtggcgcttcaagcatatatttagaggtaattgttttgtttgtagagtgtaacagaagatatcaatgaagctttatcatgtattgtaaaggtcaaaagtcatccgtctttgttttaattataacaggccaacctcggagacatttacttaccacagggtggagtacctttgtttcttcaaagaaattagtggcaggggattcttttgtattcttgaggtatctctctctggtcctagcttctcgtatgagtatgatatttgattgcattgtctatcactattgcatgtcatgtcatatatccagaacagttaatgttggtttgttaataatcatattctaatggatatgtacgctctaggggtaataatggacagctgcgagttggggtcaaaaggcttgttcgccagcagagctcaatgccgtcatcggtgatgtcaagccagagcatgcacctaggagtcttggctacagcatctcatgctgttacaacccagacaatgtttgttgtttactacaaaccgaggtttgtcacatacacctcctttcaattatcccgtcatccttacgcagtagagatcattttcatgccagagaacatactaaatctttcatatgctcttccaattttctgctttgacaaccttcagaaccactcagttcatcgtaggcgtcaacaaatacttagaggctcttaaacatgaatatgcagttggcatgcgattcaaaatgcagtttgaagccgaagggaatcctgatagaaggtagtgatatcaattactcgctagctccattttgatttatctgtcttactttcctttatagtttgtttaaaagaatgcatctttccctttttggctttaattcaacttaaattccgtgccaagttaaaaccagcaaataaattgaaatggtgggagtattcagaagtcctccaattagatgtggaagtctttgatcaagatgtttatttgccaactgcttagttctgacatgtatttgtttgggctctcccctattatttctcagatttatgggcactatagttggaattgatgatctttcttcacagtggaaaaattctgcgtggcgatccttgaaggttgatatctacatttatatttcagcaatattctttaagttcaattggaaatcaccaattctcccttcgttccaggtccgatgggacgagcctgcagccattgcaaggcctgacagagtttctccttgggaaattaaaccttatgtgtgttcaattccaaatgtccttgtcccaccaaccgcagagaagaacaaaaggcatcggctacatagtgaaatcaaaatatcaggtctgaatgaaaattctcaacagcatggtggtttttcatgtgttggttgctgatattttaatttccattttcattttcttaattatcttgcctttcttttcctgttagaacaaccttcatcctcaaatgcttcggcggtttggaatccttcccttcgatcgcctcagtttaacacctttggcatcaacagcagtactaattgcgcattagcgtctcttacagagagtggttggcagcttcctcatttaaatacttcaggtatgcttgtggatgagccagaagacggtaggagtgctccaacttggtgtggttttccatgcgttttggccccgcagttcggtcaagggactaatcagccgattgttattcctactgatgggagaaaatgtgataccaaaaaaacctgtagattatttggtattgacttgaaaagttcctcaattagcactactgaggcacgactacagctacagccagccggcatttcttgtgtctttgcagagagagcacctccaaacacggtgcctgctggtgattcagatcaaaagtctgagctttcagtagacttcaaagatcaaatgcaaggccatttgcggttacccctaaaggaggttcaaagcaagcagagttgttccaccaggtctcgcacaaaggtattaaatcaaaagccaaaaaattgcattcttaccaactcattttcgccacaaaacatattattaaactctctaacatcttttcacatgtggaatcccttgagaaggtgcaaatgcaaggcgtagctgtaggtcgtgcagtggatttaaccatattgaaaggatacgatgagcttacaaaggagcttgaggagatgtttgaaatccaaggagagcttcagtcacgacagaaatgggggatcttgtttacagatgatgaaggggatacaatgcttatgggtgattatccgtggcagtaagttctctctcacttaaaaaaatttaagactgaatacatatacaaccactgcataagatcttttctacaacctgtaatttaacacatcacttaaatcacttaattgcagagacttttgcaatgtggtgaggaagattttcatttgttcaagtcaggatatgaaaaaattgaccctgtctcgcgcagacagttaagcagctcctttcaaatgaaatgatggcggaagatggtgaaagttaaagactccgccttttgaagtggtaccgttttgtgatctctttggcttcattctctatatatagtaggaaagaacgacaggttgttgacatagcaaggcgtagaagcttgtttttacgctctttttagcctagtgcagctccgttttgagagcagttaagtaagtaactagttataataggataagactagtaaaaatatagtactagaagtagtaaaaagttaagggtgttctctaatggggagtctatatagtagtagtttatgtagttttctattcgcgcgttaacctgtaaatactcatgtgccattgtagtattctggaaatactacaatgtatggtcttgttaacgtgctttatgccttatcttatatgaaaactccatggtggagattgctctgtacttgctctttctgtatgacttttgtgatgcacaatagggatttcttgtctaaatttagactcttttggcacaatcatgcatcactttgtaactttcccctttactgaacaaaaaaatctttcaaaacgctccttcgcgtgtgtgtttgttttattctttgctaaagggtgatcctaacatttaaattagataccgttttctttactataagggtgacttgaactcaaaacctgttttattagtatgttactctatttagaagtttaataatttattagatatgacatttctatttaccttgctatttcttttaacaatatttacctttagatttatataagtttttgattcattaactgactttggttaagatgttgagtgcaaaataaatagatggttcatcttttgagctagcgtagagtcattttcctttaaactttataaagcacttacaatcacatatttgtgcattcttagtgcacgtttgaccatggataattttcactattttatggaactgtatattataatttttttggaattagaagaactcaaaaagactcttttcataattttattccgaatcactcgtacaaaaatcaaaaaacaatttcaagttgtattcattttcaagcaaaagttcaatttgtcactatcacttgtaacacaaatattttccctttcgaatttcacaattttaatattctaagttcctttttgagaacttttagtgaaaggaaaattgggctttgtgaattgtttggtcttcaatatccctatcctagctaggaagagtgtccatatagtcatgggcctcatgagttgtcaaatgttacgtaaaggtatttatctttgcatgagtgtttggtcacgctcaaaacttctttattataagtctgagtctttgtgttatgtatccatgcttggcttgcatgatgtttgtgtcattcctaatcttattgaatggactataaaggacttagtcaagtataactataaaatgacgatagcttatagacaacttcgtagttgttggagtatacaacacgctgacatcgacttcttggagtttagtaatcataattttttttaaaaaaaacaaatctcctcaatcaacttcttgattttagtagtaatccatcgattcaggtacattcatgacgtgatgtgttcgattaatattt

SEQ ID NO: 5 - DNA, 21 bp, Artificial Sequence
  primer Slactin forward
ggactctggtgatggtgttag

SEQ ID NO: 6 - DNA, 19 bp, Artificial Sequence
  primer Slactin reverse
ccgttcagcagtagtggtg

SEQ ID NO: 7 - DNA, 26 bp, Artificial Sequence
  primer SlARF9 forward
cgtaggcgtcaacaaatacttagagg

SEQ ID NO: 8 - DNA, 28 bp, Artificial Sequence
  primer SlARF9 reverse
tccactgtgaagaaagatcatcaattcc

SEQ ID NO: 9 - DNA, 27 bp, Artificial Sequence
  primer SlARF9 forward
caccatggcaactataaatgggtggtg

SEQ ID NO: 10 - DNA, 23 bp, Artificial Sequence
  primer SlARF9 reverse
ttaactgtctgcgcgagacaggg

SEQ ID NO: 11 - DNA, 36 bp, Artificial Sequence
  primer SlARF9 forward
aaaaagcaggctgtcccaccaaccgcagagaagaac

SEQ ID NO: 12 - DNA, 37 bp, Artificial Sequence
  primer SlARF9 reverse
agaaaagctgggtgctgtagtcgtgcctcagtagtgc

SEQ ID NO: 13 - DNA, 34 bp, Artificial Sequence
  primer SlARF9 promoter forward
caccttttcaaagaggtgtgacattttcaataac

SEQ ID NO: 14 - DNA, 32 bp, Artificial Sequence
  primer SlARF9 promoter reverse
caaccttcaattccaaaaactaaagaacaccc

SEQ ID NO: 15 - DNA, 24 bp, Artificial Sequence
  forward primer F-4008
aatttgagaattttggagcttttt

SEQ ID NO: 16 - DNA, 23 bp, Artificial Sequence
  reverse primer R-4009
agaacttagccttgaatcaacca

SEQ ID NO: 17 - DNA, 23 bp, Artificial Sequence
  Forward primer F-4010
tgagtttcaggtaaaaaagatgc

SEQ ID NO: 18 - DNA, 24 bp, Artificial Sequence
  reverse primer R-4011
acagaaaaaaacaacaacacagaa

SEQ ID NO: 19 - DNA, 22 bp, Artificial Sequence
  Forward primer F-4030
cccctgttttgacaagttgttg

SEQ ID NO: 20 - DNA, 26 bp, Artificial Sequence
  reverse primer R-4031
cattgatatcttctgttacactctac

SEQ ID NO: 21 - DNA, 26 bp, Artificial Sequence
  Forward primer F-4032
gtagagtgtaacagaagatatcaatg

SEQ ID NO: 22 - DNA, 25 bp, Artificial Sequence
  reverse primer R-4033
ccagagagagatacctcaagaatac

SEQ ID NO: 23 - DNA, 33 bp, Artificial Sequence
  gene-specific primer for genome walking
ttcttcagccaggaaatgactattgataactcg

SEQ ID NO: 24 - DNA, 24 bp, Artificial Sequence
  nested primer for genome walking
ggagaattcatattcggctgagac

SEQ ID NO: 25 - DNA, 22 bp, Artificial Sequence
  forward primer for kanamycin resistance gene
gactgggcacaacagacaatcg

SEQ ID NO: 26 - DNA, 24 bp, Artificial Sequence
  reverse primer for kanamycin resistance gene
gctcagaagaactcgtcaagaagg

SEQ ID NO: 27 - DNA, 34 bp, Artificial Sequence
  forward primer for AtARF9 promoter amplification
aaaaagcaggcttggtggtgggttttaaggcatc

SEQ ID NO: 28 - DNA, 37 bp, Artificial Sequence
  reverse primer for AtARF9 promoter amplification
agaaaagctgggtcacacagtctctctatctctctcc

SEQ ID NO: 29 - DNA, 30 bp, Artificial Sequence
  forward primer for AtARF9 RT-PCR
agaagccatgagcaataagttctctgtagg

SEQ ID NO: 30 - DNA, 26 bp, Artificial Sequence
  reverse primer for AtARF9 RT-PCR
gggagcagtctttcacaccaataacc

SEQ ID NO: 31 - DNA, 22 bp, Artificial Sequence
  forward primer uidA
ctcctaccgtacctcgcattac

SEQ ID NO: 32 - DNA, 19 bp, Artificial Sequence
  reverse primer uidA
ccgttgactgcctcttcgc

(19) United States
(12) Patent Application Publication
(10) Pub. No.: US 2013/0145499 A1
(43) Pub. Date: Jun. 6, 2013

(54) Plants with Increased Fruit Size

(71) Applicants: Willem Hendrik Vriezen, Haelen (NL); Celestina Mariani, Nijmegen (NL); Maaike De Jong, Trumpington (GB)

(72) Inventors: Willem Hendrik Vriezen, Haelen (NL); Celestina Mariani, Nijmegen (NL); Maaike De Jong, Trumpington (GB)

(21) Appl. No.: 13/700,684

(22) PCT Filed: May 27, 2011
(86) PCT No.: PCT/EP11/58731

§ 371 Date: Feb. 20, 2013

(30) Foreign Application Priority Data
May 28, 2010 (EP) 10005603.5

(51) Int. Cl. C12N 15/82

(52) U.S. Cl. CPC C12N 15/8262
USPC 800/290; 800/298; 800/317; 800/317.2; 800/307; 800/308; 800/317.1; 800/315; 800/320.2; 800/320.3; 800/320; 435/320.1; 800/317.4

(57) ABSTRACT

The present invention relates to the field of transgenic and non-transgenic plants with novel phenotypes. Provided are Solanum lycopersicum Auxin Response Factor 9 (SlARF9) proteins and nucleic acid sequences encoding these, which are useful in conferring novel phenotypes to plants, especially increased fruit size.

### FIELD OF THE INVENTION

The present invention relates to the field of plant biotechnology and plant breeding. Provided are plants with increased fruit size, especially tomato plants (Solanum lycopersicum) with larger and heavier tomato fruits, and methods for making genetically modified or mutant plants producing fruits having increased fruit size. The invention provides a novel use of a gene, referred to as SlARF9, encoding the SlARF9 protein, which was found to be a negative regulator of cell division during fruit development. Down-regulation, knock-out or silencing of the SlARF9 gene results in plants having significantly larger fruits at the end of the fruit growth phase. The fruits are larger due to an increase in cell division of the pericarp tissue, resulting in large fruits with more cells (and thus containing more cellulose, hemi-cellulose, pectin, etc.). Provided are also plants, seeds, fruit and plant parts, comprising a mutant SlARF9 allele (designated slarf9) herein) in their genome and having significantly larger fruits at the end of the fruit growth phase as a result of the mutation(s) in the slarf9) allele(s). In another embodiment methods for making or identifying plants comprising one or more mutant slarf9) alleles in their genome are provided herein.

### BACKGROUND OF THE INVENTION

The Angiosperms, the flowering plants are the largest group of terrestrial plants. In angiosperms, the carpel is the female reproductive organ that has differentiated into stigma, style, and ovary, which encloses the ovules. After successful completion of pollination and fertilization, the ovules develop into seeds, and the ovary develops into a fruit. The transformation from an ovary to a rapidly growing fruit includes molecular, biochemical and structural changes that must be tightly coordinated. Depending on the phase of fruit development, the temporal and spatial organization of these changes is mediated by phytohormones, such as auxin, gibberellin, cytokinin, abscisic acid and ethylene. That auxin and gibberellin also act as important factors early in the initiation of fruit development was already established in the early 20th century. However, to date, the complex regulatory network that is controlled by these hormones is still poorly understood.

“Fruit set” is defined as the transition of a quiescent ovary to a rapidly growing young fruit, which is an important process in the sexual reproduction of flowering plants. The tomato (Solanum lycopersicum L.) is one of the most studied fleshy fruits, representing the Solanaceae, a family that contains several other important fruit crops, such as the eggplant (Solanum melongena L.) and peppers (Capsicum spp.). The biology of tomato is highly favourable. It has a relatively short life cycle, has uncomplicated requirements for growth and maintenance, and although tomato is a self-pollinator, it is easy to cross-pollinate. Furthermore, a wide range of genetic resources, such as phenotypic divergent cultivars, intercrossable wild relatives, mutants, and genomic tools (Mueller et al., Plant Physiology 138, 1310-1317; Mueller et al., Comparative and Functional Genomics 6,

153-158), such as BAC libraries and expressed sequence tags (ESTs), are available.

Tomato fruit set is very sensitive to environmental conditions, in particular, to too low or too high temperatures that affect pollen development and anther dehiscence. Adams et al. (2001, Annals of Botany 88, 869-877) showed that a constant temperature regime of 14° C. or 26° C. strongly reduced tomato fruit set as compared to a regime of 22° C. Nevertheless, the optimum growth temperature may vary, depending on the cultivar. As a consequence of this temperature-sensitivity, efficient tomato production is restricted to certain climatic zones. For this reason, tomato seed companies breed at different places in the world to develop cultivars suited for optimal fruit production under the local climate conditions. Nevertheless, even with these optimized lines it is often not possible to grow tomatoes during the summer in warm regions such as the Southern parts of Europe. In the more Northern parts, tomato production is only possible during the warm season, and even then only in modern greenhouses at the expense of a huge amount of energy for heating.

Fruit set depends on the successful completion of pollination and fertilization (Gillaspy et al., 1993, The Plant Cell 5, 1439-1451). When the flower of tomato is fully opened at the stage of anthesis, compatible pollen has to germinate on the pistil and form a pollen tube. This pollen tube then grows through the style and the ovular micropyle to deliver two sperm cells in the embryo sac. There a double fertilization occurs; one of the two sperm cells fertilizes the egg cell, while the other fuses with two haploid polar nuclei in the central cell. Consequently, both embryo and surrounding tissues may generate signals that stimulate fruit growth. The tomato ovary is composed of two or more carpels, which enclose the locular cavities containing the ovules. After successful fertilization, the development of the ovary into a fruit starts with a period of cell division which continues for 10-14 days. During the following 6-7 weeks, fruit growth mainly depends on cell expansion (Mapelli et al., 1978, Plant and Cell Physiology 19, 1281-1288; Bünger-Kibler and Bangerth, 1982, Plant Growth Regulation 1, 143-154; Gillaspy et al., 1993, supra). The carpel wall develops into the pericarp, and the placenta, to which the ovules are attached, develops into a gel-like substance, consisting of large, thin-walled cells that are highly vacuolated. At the end of the cell-expansion period, the fruit has reached its final size and will start to ripen (Gillaspy et al., 1993, supra). Six ripening stages are differentiated: immature, mature, mature green, breaker, pink and red. Processing tomatoes are generally harvested at the red stage, while tomatoes for the fresh market are harvested earlier, either at the breaker stage (which do not need to be treated with ethylene to ripen to the red stage) or green mature stage (which need exposure to ethylene gas to ripen through to the red stage).

Although the influence of phytohormones, such as auxin and gibberellin, over fruit development was already acknowledged back in the early 20^{th} century (Gustafson, 1937, American Journal of Botany 24, 102-107; Gustafson 1939 American Journal of Botany 26, 135-138; Wittwer et al., 1957, Plant Physiology 32, 39-41), the molecular mechanisms that underlie fruit set are still largely unknown and are now starting to be unravelled.

Auxin acts as an important regulator in a wide range of developmental processes throughout a plant's life cycle by affecting the expression of many genes (Theologis, 1986, Annual Reviews of Plant Physiology 37, 407-438). This auxin-mediated gene expression is controlled by two families of transcription factors, the Auxin Response Factors (ARFs) and the Auxin/Indole-3-Acetic Acids (Aux/IAAs), which are represented by two large gene families in plant species such as Arabidopsis and rice (Hagen and Guilfoyle, 2002, Plant Molecular Biology 49, 373-385, Plant Molecular Biology 49, 387-400; Liscum and Reed, 2002, infra; Wang et al., 2007, Gene 394, 13-24). The proteins encoded by these families share two conserved C-terminal domains, the domains III and IV that serve as interaction domains between the Aux/IAAs and ARFs, which allow interaction between ARFs and Aux/IAAs with the formation of homo- or heterodimers, respectively (Kim et al., 1997, Proceedings of the National Academy of Sciences, USA 94, 11786-11791; Ulmasov et al., 1997, Science 276, 1865-1868; Ulmasov et al., 1999, The Plant Journal 19, 309-319). Furthermore, the ARFs contain an N-terminal B3-derived DNA binding domain (DBD) that binds the Auxin Response Elements (AuxRE) in the promoter regions of auxin-regulated genes (Ulmasov et al., 1999 supra), and a middle region (MR), which functions as a transcriptional activation or repression domain depending on its amino acid composition (Ulmasov et al., 1999, Proceedings of the National Academy of Sciences, USA 96, 5844-5849; Tiwari et al., 2003, The Plant Cell 15, 533-543). The Aux/IAA proteins act as repressors by blocking the transcriptional activity of ARFs (Liscum and Reed, 2002, Plant Molecular Biology 49, 387-400). The repressing activity of the Aux/IAAs is conferred by the N-terminal domain I (Tiwari et al., 2004, The Plant Cell 16, 533-543). Recently, Szemenyei et al. (2008, Science 319, 1384-1386) have shown that in a number of Aux/IAAs, this domain contains an ERF-associated amphiphilic repression (EAR) motif that recruits TOPLESS (TPL), a transcriptional corepressor. Additionally, the Aux/IAAs contain a fourth conserved region, the domain II (Tiwari et al., 2001, The Plant Cell 13, 2809-2822). Auxin enhances the interactions between this domain and the SCFTIR1 ubiquitin ligase complex, containing the F-box auxin receptor protein TIR1 (TRANSPORT INHIBITOR RESISTANT1), resulting in the ubiquitin-mediated degradation of the Aux/IAAs (Dharmasiri et al., 2005, Nature 435, 441-445; Kepinski and Leyser, 2005, Nature 435, 446-451; Tan et al., 2007, Nature 446, 640-645; dos Santos Maraschin et al., 2009, The Plant Journal 59, 100-109). Consequently, the ARFs are released from repression, resulting in transcription of auxin response genes (Woodward and Bartel, 2005, Annals of Botany 95, 707-735). However, this model only supports the function of transcriptional activating ARFs.

The mechanism by which ARF repressors regulate the expression of auxin-dependent genes is still unclear, since their interactions with Aux/IAAs, or with activating ARFs are very weak (Tiwari et al., 2003, supra; Hardtke et al., 2004, Development 131, 1089-1100). Alternatively, the ARF repressors may compete with the ARF activators for the AuxRE binding sites in the promoters of the auxin response genes, thus inhibiting the expression of these genes independently of Aux/IAAs and providing an alternative mechanism of gene regulation (Guilfoyle and Hagen, 2007, Plant Biology 10, 453-460).

In tomato, auxin plays an important role in fruit set and fruit development. Iwahori (1967, Plant and Cell Physiology 8, 15-22) and Mapelli et al. (1978, Plant and Cell Physiology 19, 1281-1288) showed that the auxin concentration in the ovary rapidly increased after pollination, reaching its maximum 7-8 days after pollination (DAP). A second peak of auxin activity was observed 30 DAP. The importance of auxin in tomato fruit set was demonstrated by the ovary-specific expression of the iaaM or rolB genes from Agrobacterium spp., affecting auxin synthesis or responsiveness, which resulted in the formation of seedless (parthenocarpic) tomato fruits (Ficcadenti et al., 1999, Molecular Breeding 5, 463-470; Carmi et al., 2003, Planta 217, 726-735). Also the application of auxin on unpollinated ovaries led to the formation of fruits without the need for pollination and fertilization (Gustafson, 1936, Proceedings of the National Academy of Sciences, USA 22, 628-636; Bünger-Kibler and Bangerth, 1982, supra). Normally, during the first 10-14 days (after fertilization) of development, tomato fruit growth mainly depends on cell division. During the following 6-7 weeks, fruit growth essentially depends on cell expansion (Mapelli et al., 1978, supra; Bünger-Kibler and Bangerth, 1982, supra; Gillaspy et al., 1993, supra). However, in fruits induced by the auxin indole-3-acetic acid (IAA) the period of cell division was shorter, only lasting 10 days, although cell division took place at a higher rate as compared to that in seeded control fruits. Nevertheless, these IAA-induced fruits remained smaller than control fruits as cell expansion was strongly impaired (Bünger-Kibler and Bangerth, 1982, supra). Treatments with synthetic auxins stimulated cell division for an extended period, resulting in the formation of fruits with a higher number of pericarp cells (Bünger-Kibler and Bangerth, 1982, supra; Serrani et al., 2007, Journal of Plant Growth Regulation 26, 211-221). These findings suggest that during the early stages of tomato fruit development, cell division activity is tightly regulated by auxin.

Previously, cDNA-amplified fragment length polymorphism based transcript profiling (cDNA-AFLP) was used to identify genes that are differentially expressed during fruit set (Vriezen et al., 2008, New Phytologist 177, 60-76). One of the 874 genes induced in the ovaries by pollination had some sequence similarity to Arabidopsis thaliana ARF9 (AtARF9) and GenBank accession number BT013639.1 (a tomato clone with unknown gene function). Despite its not very high amino acid identity to AtARF9 (52% sequence identity using the pairwise sequence alignment program Emboss “Needle”), the gene was named SlARF9 (Solanum lycopersicum ARF9).

Arabidopsis thaliana has 23 ARF genes. AtARF9 is thought to be involved in the gravitropic signal transduction because an Arabidopsis arf9 mutant line, lacking the 3′-end of the transcript, over-responded after gravistimulation (Roberts et al., 2007, Gravitational and Space Biology Bulletin 20, 103-104). Furthermore, the AtARF9 gene was found to be expressed in the suspensor of the Arabidopsis embryo, and double knock-out lines, in which both ARF9 and ARF13 were silenced, showed that AtARF9 is necessary for the control of suspensor development (Liu et al., 2008, 19th International Conference on Arabidopsis Research, Montreal, Canada). So far, the only ARF known to be involved in the fruit development process is FRUIT WITHOUT FERTILIZATION (FWF)/ARF8, as fwf/arf8 mutant lines form parthenocarpic siliques (Goetz et al., 2006, The Plant Cell 18, 1873-1886). In tomato, transgenic lines with reduced SlARF7 transcript levels also form parthenocarpic fruits, which indicate that SlARF7 acts as a negative regulator of fruit set (de Jong et al., 2009, The Plant Journal 57, 160-170). The only other member of the tomato ARF family characterized so far is the DEVELOPMENTALLY REGULATED GENE 12 (DR12), the homologue of AtARF4. The mRNA levels of DR12 increased throughout fruit development, and reached the highest level at the early red-stage fruit. Down-regulation of this gene by anti-sense approach affected the fruit firmness at the red stage (Jones et al., 2002, The Plant Journal 32, 603-613).

Despite the fact that sequence similarity of SlARF9 to AtARF9 might suggest that these genes could potentially be orthologs (although sequence similarity is low), SlARF9 has by the present inventors been found to have a completely different function than AtARF9 and to be expressed in different tissues than AtARF9. The present inventors found that the SlARF9 gene encodes a transcription factor protein which negatively regulates cell division during the fruit growth. Transgenic tomato plants in which SlARF9 mRNA transcript level were reduced by RNAi (gene silencing) formed significantly bigger and heavier fruits than wild type control lines. In contrast, SlARF9 overexpressing lines had significantly smaller and lighter fruits than the wild type. Further, the number of cells and the number of cell layers in the pericarp was significantly higher in the SlARF9-RNAi fruits compared to wild type fruits. Larger fruits with more, smaller cells have the benefit of a relative increase in not only yield, but also in solid components (cell walls, comprising pectin, hemi-cellulose and cellulose).

### GENERAL DEFINITIONS

The term “nucleic acid sequence” (or nucleic acid molecule) refers to a DNA or RNA molecule in single or double stranded form, particularly a DNA encoding a protein or protein fragment according to the invention. An “isolated nucleic acid sequence” refers to a nucleic acid sequence which is no longer in the natural environment from which it was isolated, e.g. the nucleic acid sequence in a bacterial host cell or in the plant nuclear or plastid genome.

The terms “protein” or “polypeptide” are used interchangeably and refer to molecules consisting of a chain of amino acids, without reference to a specific mode of action, size, 3 dimensional structure or origin. A “fragment” or “portion” of a SlARF9 protein may thus still be referred to as a “protein”. An “isolated protein” is used to refer to a protein which is no longer in its natural environment, for example in vitro or in a recombinant bacterial or plant host cell. The term “gene” means a DNA sequence comprising a region (transcribed region), which is transcribed into an RNA molecule (e.g. an mRNA or an RNAi molecule) in a cell, operably linked to suitable regulatory regions (e.g. a promoter). A gene may thus comprise several operably linked sequences, such as a promoter, a 5′ leader sequence comprising e.g. sequences involved in translation initiation, a (protein) coding region (cDNA or genomic DNA) and a 3′ non-translated sequence comprising e.g. transcription termination sites.

A “chimeric gene” (or recombinant gene) refers to any gene, which is not normally found in nature in a species, in particular a gene in which one or more parts of the nucleic acid sequence are present that are not associated with each other in nature. For example the promoter is not associated in nature with part or all of the transcribed region or with another regulatory region. The term “chimeric gene” is understood to include expression constructs in which a promoter or transcription regulatory sequence is operably linked to one or more coding sequences or to an antisense (reverse complement of the sense strand) or inverted repeat sequence (sense and antisense, whereby the RNA transcript forms double stranded RNA upon transcription). A “cis-gene” is a chimeric gene wherein preferably all of the gene sequences, but at least the transcribed sequence, are/is from a plant species which is sexually compatible with the species into which the gene is introduced.

“Expression of a gene” refers to the process wherein a DNA region, which is operably linked to appropriate regulatory regions, particularly a promoter, is transcribed into an RNA, which is biologically active, i.e. which is capable of being translated into a biologically active protein or peptide (or active peptide fragment) or which is active itself (e.g. in posttranscriptional gene silencing or RNAi). The coding sequence may be in sense-orientation and encodes a desired, biologically active protein or peptide, or an active peptide fragment. In gene silencing approaches, the DNA sequence is preferably present in the form of an antisense DNA or an inverted repeat DNA, comprising a short sequence of the target gene in antisense or in sense and antisense orientation (inverted repeat). “Ectopic expression” refers to expression in a tissue in which the gene is normally not expressed.

An “active protein” or “functional protein” is a protein which has protein activity as measurable in vitro, e.g. by an in vitro activity assay, and/or in vivo, e.g. by the phenotype conferred by the protein. A “wild type” protein is a fully functional protein, as present in the wild type plant. A “mutant protein” is herein a protein comprising one or more mutations in the nucleic acid sequence encoding the protein, whereby the mutation results in (the mutant nucleic acid molecule encoding) a “reduced-function” or “loss-of-function” protein, as e.g. measurable in vivo, e.g. by the phenotype conferred by the mutant allele.

A “mutation” in a nucleic acid molecule coding for a protein is a change of one or more nucleotides compared to the wild type sequence, e.g. by replacement, deletion or insertion of one or more nucleotides. A “point mutation” is the replacement of a single nucleotide, or the insertion or deletion of a single nucleotide.

A “non-sense” mutation is a (point) mutation in a nucleic acid sequence encoding a protein, whereby a codon is changed into a stop codon. This results in a premature stop codon being present in the mRNA and in a truncated protein. A truncated protein may have reduced function or loss of function.

A “mis-sense” mutation is a (point) mutation in a nucleic acid sequence encoding a protein, whereby a codon is changed to code for a different amino acid. The resulting protein may have reduced function or loss of function.

A “splice-site” mutation is a mutation in a nucleic acid sequence encoding a protein, whereby RNA splicing of the pre-mRNA is changed, resulting in an mRNA having a different nucleotide sequence and a protein having a different amino acid sequence than the wild type. The resulting protein may have reduced function or loss of function.

A “frame-shift” mutation is a mutation a nucleic acid sequence encoding a protein by which the reading frame of the mRNA is changed, resulting in a different amino acid sequence. The resulting protein may have reduced function or loss of function.

A mutation in a regulatory sequence, e.g. in a promoter of a gene, is a change of one or more nucleotides compared to the wild type sequence, e.g. by replacement, deletion or insertion of one or more nucleotides, leading for example to reduced or no mRNA transcript of the gene being made.

“Silencing” refers to a down-regulation or complete inhibition of gene expression of the target gene or gene family.

A “target gene” in gene silencing approaches is the gene or gene family (or one or more specific alleles of the gene) of which the endogenous gene expression is down-regulated or completely inhibited (silenced) when a chimeric silencing gene (or ‘chimeric RNAi gene’) is expressed and for example produces a silencing RNA transcript (e.g. a dsRNA or hairpin RNA capable of silencing the endogenous target gene expression). In mutagenesis approaches, a target gene is the endogenous gene which is to be mutated, leading to a change in (reduction or loss of) gene expression or a change in (reduction or loss of) function of the encoded protein.

A “sense” RNA transcript is generally made by operably linking a promoter to a double stranded DNA molecule wherein the sense strand (coding strand) of the DNA molecule is in 5′ to 3′ orientation, such that upon transcription a sense RNA is transcribed, which has the identical nucleotide sequence to the sense DNA strand (except that T is replaced by U in the RNA). An “antisense” RNA transcript is generally made by operably linking a promoter to the complementary strand (antisense strand) of the sense DNA, such that upon transcription an antisense RNA is transcribed.

A “transcription regulatory sequence” is herein defined as a nucleic acid sequence that is capable of regulating the rate of transcription of a (coding) sequence operably linked to the transcription regulatory sequence. A transcription regulatory sequence as herein defined will thus comprise all of the sequence elements necessary for initiation of transcription (promoter elements), for maintaining and for regulating transcription, including e.g. attenuators or enhancers. Although mostly the upstream (5′) transcription regulatory sequences of a coding sequence are referred to, regulatory sequences found downstream (3′) of a coding sequence are also encompassed by this definition.

As used herein, the term “promoter” refers to a nucleic acid fragment that functions to control the transcription of one or more genes, located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically (e.g. by external application of certain compounds) or developmentally regulated. A “tissue specific” promoter is only active in specific types of tissues or cells. A “tissue-preferred” promoter is mainly active in certain tissues (e.g. developing fruit tissue), but which may also have some activity in other tissues.

As used herein, the term “operably linked” refers to a linkage of polynucleotide elements in a functional relationship. A nucleic acid is “operably linked” when it is placed into a functional relationship with another nucleic acid sequence. For instance, a promoter, or rather a transcription regulatory sequence, is operably linked to a coding sequence if it affects the transcription of the coding sequence. Operably linked means that the DNA sequences being linked are typically contiguous and, where necessary to join two protein encoding regions, contiguous and in reading frame so as to produce a “chimeric protein”. A “chimeric protein” or “hybrid protein” is a protein composed of various protein “domains” (or motifs) which is not found as such in nature but which a joined to form a functional protein, which displays the functionality of the joined domains. A chimeric protein may also be a fusion protein of two or more proteins occurring in nature.

The term “domain” as used herein means any part(s) or domain(s) of the protein with a specific structure or function that can be transferred to another protein for providing a new hybrid protein with at least the functional characteristic of the domain. Specific domains can also be used to identify protein members belonging to the SlARF9 group of proteins, such as SlARF9 variants from tomato plants or SlARF9 orthologs from other plant species. Examples of domains found in SlARF9 proteins are the B3-derived DNA-binding domain comprising about amino acid 74-236 of SEQ ID NO: 2 or variants thereof, the Middle Region (MR) comprising about amino acids 237-564 of SEQ ID NO: 2 or variants thereof, the Auxin Response Region comprising about amino acids 256-332 of SEQ ID NO: 2 or variants thereof, or the Dimerization Domains III or IV comprising about amino acids 565-602 or 609-651 of SEQ ID NO: 2, respectively, or variants thereof.

The terms “target peptide” refers to amino acid sequences which target a protein to intracellular organelles such as plastids, preferably chloroplasts, mitochondria, or to the extracellular space (secretion signal peptide). A nucleic acid sequence encoding a target peptide may be fused (in frame) to the nucleic acid sequence encoding the amino terminal end (N-terminal end) of the protein.

A “nucleic acid construct” or “vector” is herein understood to mean a man-made nucleic acid molecule resulting from the use of recombinant DNA technology and which is used to deliver exogenous DNA into a host cell. The vector backbone may for example be a binary or superbinary vector (see e.g. U.S. Pat. No. 5,591,616, US2002138879 and WO9506722), a co-integrate vector or a T-DNA vector, as known in the art and as described elsewhere herein, into which a chimeric gene is integrated or, if a suitable transcription regulatory sequence is already present, only a desired nucleic acid sequence (e.g. a coding sequence, an antisense or an inverted repeat sequence) is integrated downstream of the transcription regulatory sequence. Vectors usually comprise further genetic elements to facilitate their use in molecular cloning, such as e.g. selectable markers, multiple cloning sites and the like (see below).

A “host cell” or a “recombinant host cell” or “transformed cell” are terms referring to a new individual cell (or organism) arising as a result of at least one nucleic acid molecule, especially comprising a chimeric gene encoding a desired protein or a nucleic acid sequence which upon transcription yields an antisense RNA or an inverted repeat RNA (or hairpin RNA) for silencing of a target gene/gene family, having been introduced into said cell. The host cell is preferably a plant cell or a bacterial cell. The host cell may contain the nucleic acid construct as an extra-chromosomally (episomal) replicating molecule, or more preferably, comprises the chimeric gene integrated in the nuclear or plastid genome of the host cell.

The term “selectable marker” is a term familiar to one of ordinary skill in the art and is used herein to describe any genetic entity which, when expressed, can be used to select for a cell or cells containing the selectable marker. Selectable marker gene products confer for example antibiotic resistance, or more preferably, herbicide resistance or another selectable trait such as a phenotypic trait (e.g. a change in pigmentation) or a nutritional requirements. The term “reporter” is mainly used to refer to visible markers, such as green fluorescent protein (GFP), eGFP, luciferase, GUS and the like.

The term “ortholog” of a gene or protein refers herein to the homologous gene or protein found in another species, which has the same function as the gene or protein, but (usually) diverged in sequence from the time point on when the species harbouring the genes diverged (i.e. the genes evolved from a common ancestor by speciation). Orthologs of the tomato SlARF9 gene may thus be identified in other plant species based on both sequence comparisons (e.g. based on percentages sequence identity over the entire sequence and/or over specific domains) and/or functional analysis.

The terms “homologous” and “heterologous” refer to the relationship between a nucleic acid or amino acid sequence and its host cell or organism, especially in the context of transgenic organisms. A homologous sequence is thus naturally found in the host species (e.g. a tomato plant transformed with a tomato gene), while a heterologous sequence is not naturally found in the host cell (e.g. a tomato plant transformed with a sequence from potato plants). Depending on the context, the term “homolog” or “homologous” may alternatively refer to sequences which are descendent from a common ancestral sequence (e.g. they may be orthologs).

“Stringent hybridisation conditions” can be used to identify nucleotide sequences, which are substantially identical to a given nucleotide sequence. Stringent conditions are sequence dependent and will be different in different circumstances. Generally, stringent conditions are selected to be about 5° C. lower than the thermal melting point (Tₘ) for the specific sequences at a defined ionic strength and pH. The Tₘ is the temperature (under defined ionic strength and pH) at which 50% of the target sequence hybridises to a perfectly matched probe. Typically stringent conditions will be chosen in which the salt concentration is about 0.02 molar at pH 7 and the temperature is at least 60° C. Lowering the salt concentration and/or increasing the temperature increases stringency. Stringent conditions for RNA-DNA hybridisations (Northern blots using a probe of e.g. 100 nt) are for example those which include at least one wash in 0.2×SSC at 63° C. for 20 min, or equivalent conditions. Stringent conditions for DNA-DNA hybridisation (Southern blots using a probe of e.g. 100 nt) are for example those which include at least one wash (usually 2) in 0.2×SSC at a temperature of at least 50° C., usually about 55° C., for 20 min, or equivalent conditions. See also Sambrook et al. (1989) and Sambrook and Russell (2001).

“Sequence identity” and “sequence similarity” can be determined by alignment of two peptide or two nucleotide sequences using global or local alignment algorithms. Sequences may then be referred to as “substantially identical” or “essentially similar” when they (when optimally aligned by for example the programs GAP or BESTFIT or the Emboss program “Needle” (using default parameters, see below) share at least a certain minimal percentage of sequence identity (as defined further below). These programs use the Needleman and Wunsch global alignment algorithm to align two sequences over their entire length, maximizing the number of matches and minimises the number of gaps. Generally, the default parameters are used, with a gap creation penalty=10 and gap extension penalty=0.5 (both for nucleotide and protein alignments). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may for example be determined using computer programs, such as the GCG Wisconsin Package, Version 10.3, available from Accelrys Inc., 9685 Scranton Road, San Diego, Calif. 92121-3752 USA or EMBOSS (http://www.ebi.ac.uk/Tools/webservices/services/emboss). Alternatively percent similarity or identity may be determined by searching against databases such as FASTA, BLAST, etc., but hits should be retrieved and aligned pairwise to compare sequence identity.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”. It is further understood that, when referring to “sequences” herein, generally the actual physical molecules with a certain sequence of subunits (e.g. amino acids) are referred to.

As used herein, the term “plant” includes the whole plant or any parts or derivatives thereof, such as plant organs (e.g., harvested or non-harvested storage organs, bulbs, tubers, fruits, leaves, etc.), plant cells, plant protoplasts, plant cell or tissue cultures from which whole plants can be regenerated, plant calli, plant cell clumps, and plant cells that are intact in plants, or parts of plants, such as embryos, pollen, ovules, ovaries, fruits (e.g., harvested tissues or organs, such as harvested tomatoes or parts thereof), flowers, leaves, seeds, tubers, bulbs, clonally propagated plants, roots, root-stocks, stems, root tips and the like. Also any developmental stage is included, such as seedlings, immature and mature, etc.

“Plant variety” is a group of plants within the same botanical taxon of the lowest grade known, which (irrespective of whether the conditions for the recognition of plant breeder's rights are fulfilled or not) can be defined on the basis of the expression of characteristics that result from a certain genotype or a combination of genotypes, can be distinguished from any other group of plants by the expression of at least one of those characteristics, and can be regarded as an entity, because it can be multiplied without any change. Therefore, the term “plant variety” cannot be used to denote a group of plants, even if they are of the same kind, if they are all characterized by the presence of 1 locus or gene (or a series of phenotypical characteristics due to this single locus or gene), but which can otherwise differ from one another enormously as regards the other loci or genes.

“F1, F2, etc.” refers to the consecutive related generations following a cross between two parent plants or parent lines. The plants grown from the seeds produced by crossing two plants or lines is called the F1 generation. Selfing the F1 plants results in the F2 generation, etc. “F1 hybrid” plant (or F1 seed) is the generation obtained from crossing two inbred parent lines. An “M1 population” is a plurality of mutagenized seeds/plants of a certain plant line or cultivar. “M1, M2, M3, M4, etc.” refers to the consecutive generations obtained following selfing of a first mutagenized seed/plant (M1).

The term “allele(s)” means any of one or more alternative forms of a gene at a particular locus, all of which alleles relate to one trait or characteristic at a specific locus. In a diploid cell of an organism, alleles of a given gene are located at a specific location, or locus (loci plural) on a chromosome. One allele is present on each chromosome of the pair of homologous chromosomes. A diploid plant species may comprise a large number of different alleles at a particular locus. These may be identical alleles of the gene (homozygous) or two different alleles (heterozygous).

The term “locus” (loci plural) means a specific place or places or a site on a chromosome where for example a gene or genetic marker is found. The SlARF9 locus is thus the location in the genome where the SlARF9 gene is found. Without limiting the invention, the SlARF9 locus is thought to be located in chromosome 8 of the tomato genome.

“Wild type allele” (WT) refers herein to a version of a gene encoding a functional protein (wild type protein). The wild type SlARF9 allele of tomato cultivar Moneymaker is, for example, depicted in SEQ ID NO: 1 (mRNA/cDNA) and in SEQ ID NO: 3 (genomic DNA, with the SlARF9 coding region ranging from nucleotide 2005 to 5879). The wild type SlARF9 allele of tomato cultivar Heinz 1706 is depicted in SEQ ID NO: 4 (genomic DNA, with the SlARF9 coding region ranging from nucleotide 1196 to 5869). “Mutant allele” refers herein to an allele comprising one or more mutations in the coding sequence (mRNA, cDNA or genomic sequence) compared to the wild type allele. Such mutation(s) (e.g. insertion, inversion, deletion and/or replacement of one or more nucleotides) may lead to the encoded protein having reduced in vitro and/or in vivo functionality (reduced function) or no in vitro and/or in vivo functionality (loss of function), e.g. due to the protein e.g. being truncated or having an amino acid sequence wherein one or more amino acids are deleted, inserted or replaced. Such changes may lead to the protein having a different 3D conformation, being targeted to a different sub-cellular compartment, having a modified catalytic domain, having a modified binding activity to nucleic acids or proteins, etc.

“Larger fruit size” or “significantly enhanced fruit size”, “significantly larger fruit size” or “plant producing significantly larger fruits” refers herein to the fruit size being (on average) significantly larger compared to suitable control plants (e.g. wild type plants), i.e. the average equatorial diameter and/or average volume and/or average fresh fruit weight being significantly higher than in the controls. The fruit size is preferably determined at the end of the fruit growth phase (i.e. when the fruit has reached its final size) or thereafter (in full-sized fruit, e.g. at breaker stage).

“More pericarp cells” or “fruits with more cells” refers herein to the number of pericarp cells and/or the number of cell layers in the pericarp (including epidermis, exocarp, mesocarp and endocarp cell layers) being significantly higher (on average) than in the controls. The cell number is preferably determined at the end of the cell division phase (e.g. at or after about 10 DAP in tomato) and/or at the end of the fruit growth phase or thereafter (in full-sized fruit, e.g. at breaker stage in tomato).

“Smaller pericarp cells” refers to the average cell size of the pericarp cells, especially of the mesocarp cells, being significantly smaller than in the controls (e.g. wild type fruits). Cell size number is preferably determined at the end of the cell division phase (e.g. at or after about 10 DAP in tomato) and/or at the end of the fruit growth phase or thereafter (in full-sized fruit, e.g. at breaker stage).

“Wild type plant” and “wild type fruits” refers herein to a plant comprising a wild type (WT) SlARF9 allele encoding a functional protein (e.g. in contrast to “mutant plants”, comprising a mutant slarf9) allele). Such plants are for example suitable controls in phenotypic assays. Preferably wild type and/or mutant plants are “cultivated plants”, i.e. varieties, breeding lines or cultivars of a species, cultivated by humans and having good agronomic characteristics; preferably such plants are not “wild plants”, i.e. plants which generally have much poorer yields and poorer agronomic characteristics than cultivated plants and e.g. grow naturally in wild populations. “Wild plants” include for example ecotypes, PI (Plant Introduction) lines, landraces or wild accessions or wild relatives of a species, or so-called heirloom varieties or cultivars, i.e. varieties or cultivars commonly grown during earlier periods in human history, but which are not used in modern agriculture.

“Variants” of the SlARF9 gene or protein include both natural allelic variants found within the species S. lycopersicum or in wild relatives of tomato, as well as orthologs found in other plant species, such as other dicotyledonous plant species, or monocotyledonous species.

Wild relatives of tomato include S. arcanum, S. chmielewskii, S. neorickii (=L. parviflorum), S. cheesmaniae, S. galapagense, S. pimpinellifolium, S. chilense, S. corneliomulleri, S. habrochaites (=L. hirsutum), S. huaylasense, S. sisymbriifolium, S. peruvianum, S. hirsutum or S. pennellii.

“Average” refers herein to the arithmetic mean.

### DETAILED DESCRIPTION OF THE INVENTION

The present inventors set out to study genes which are differentially expressed during tomato fruit set, by carrying out transcriptome analysis (cDNA-AFLP) of pollinated ovaries and GA₃ (gibberellic acid) treated ovaries (Vriezen et al. 2008, New Phytologist 177:60-76). One gene, which was highly expressed in pollinated ovaries was characterized further by generating transgenic plants, in order to study the role of this gene. The tomato gene was named SlARF9 (Solanum lycopersicum ARF9), as it encodes a protein of 658 amino acids which comprises an amino acid sequence most similar to the Arabidopsis ARF9 protein (52% amino acid sequence identity and 61% cDNA sequence identity, using the Emboss program ‘needle’, GAP opening=10 and GAP extension=0.5).

It was found that SlARF9 is highly expressed in ovules, placenta and pericarp of pollinated ovaries (see FIG. 1). More detailed analysis showed that SlARF9 was transcribed in other plant tissues as well, such as the axillary meristems and root meristems. In general, these are tissues in which many cell divisions occur. Transgenic plants with increased SlARF9 mRNA levels formed fruits that were smaller than wild-type fruits. While the fruits of transgenic lines in which SlARF9 mRNA levels were reduced formed bigger fruits due to increased cell division activity. The expression analysis, together with the phenotype of the transgenic lines, suggested that SlARF9 acts as a repressor of cell division during fruit development. Notably, reducing SlARF9 messenger RNA by expressing the RNAi molecule constitutively, under control of the CaMV 35S promoter, did not cause any other negative phenotypes. Thus, despite the fact that the SlARF9 gene cannot be considered a fruit-specific gene, the RNAi SlARF9 lines only displayed a fruit phenotype, indicating that in other plant tissues SlARF9 may act redundantly with other members of the ARF protein family.

The finding that SlARF9 is involved in fruit size can be used to generate transgenic and/or non-transgenic plants with larger fruits by either reducing the amount of wild type SlARF9 protein (or variants or orthologs thereof) and/or the functioning of wild type protein (or variants or orthologs thereof) during fruit growth, as will be described further below. In particular, cell division during fruit growth is hereby enhanced, leading to significantly more cells and/or significantly more cell layers in the pericarp and/or significantly smaller cells in the fruit. Plants, thus, produce larger fruits with more solid components.

The finding can also be used to significantly decrease fruit size compared to the control (e.g. the wild type plant) by overexpressing a SlARF9 gene, variant or ortholog, encoding a functional SlARF9 protein (or variant) in a plant as described elsewhere herein.

The different embodiments of the invention are described herein below and in the non-limiting Examples. Parts described herein as being applicable to transgenic approaches are generally also applicable to non-transgenic approaches and vice versa, unless indicated otherwise.

In one embodiment transgenic plants in which endogenous SlARF9 expression is down-regulated or silenced, at least during fruit growth, and which produce significantly larger fruit are provided. In another embodiment non-transgenic plants comprising one or more mutant slarf9 alleles (either in homozygous or heterozygous form) and wherein said mutant allele(s) encode(s) an SlARF9 protein which has reduced functionality in vitro and/or in vivo compared to the wild type protein, or even no functionality (e.g. through a translation stop codon or frame-shift mutation), and whereby the mutation results in the plants (mutant line or progeny thereof) having significantly larger fruits compared to plants lacking the mutant allele(s) (wild type plants), are provided herein. Such non-transgenic, large fruit producing plants are in one embodiment of the invention generated or identified using TILLING or Eco-TILLING, but can also be generated or identified using other known mutagenesis methods combined with breeding methods. Thus, in one embodiment the mutant slarf9 allele is induced and/or identified by humans, using mutagenesis techniques (“induced mutant”), while in another embodiment of the invention the mutant slarf9 allele is a “natural mutant”, meaning it is identified in natural plant populations (such as wild relatives of tomato) and is then introduced into elite germplasm. “Induced mutants” are preferably generated in cultivated germplasm and are thus directly present in agronomically valuable lines. On the other hand “natural mutants” or “spontaneous mutants” or “natural variants” or “natural allelic variants/variation” are based on natural variation (polymorphisms/mutations) found in a species and are, thus, likely present in plant material of inferior agronomic quality, not cultivated in modern agriculture, e.g. wild plants. The later alleles then need to be transferred into a cultivated plant having good agronomic characteristics, which is an embodiment of the invention.

### Nucleic Acid Sequences and Proteins for Uses According to the Invention

In one embodiment of the invention nucleic acid sequences and amino acid sequences of SlARF9 are provided, as well as methods for isolating or identifying “variants” thereof for example allelic variants within the species (Solanum lycopersicum) or within the genus Solanum (e.g. wild tomato relative species such as S. pennelli, S. habrochaites, etc.), or orthologs of SlARF9 of other plant species, such as other vegetable species (e.g. species in the family Solanaceae, e.g. pepper or eggplant; or Cucurbitaceae, e.g. melon, watermelon or cucumber) or field crop species (e.g. corn, wheat, rice).

The wild type SlARF9 transcription factor protein derived from tomato cultivar Moneymaker (fresh market tomato) is depicted in SEQ ID NO: 2. It is a protein of 658 amino acids which comprises several domains, namely a) a DNA-binding domain located in the N-terminal region (amino acids 74-236 of SEQ ID NO: 2), which likely is capable to bind to cis-regulatory elements in the promoter region of auxin-regulated genes, b) a middle region (“MR”, amino acids 237 to 564 of SEQ ID NO: 2) and c) two dimerization domains, domain III (amino acids 565-602 of SEQ ID NO: 2) and domain IV (amino acids 609 to 651 of SEQ ID NO: 2).

The protein of processing tomato cultivar Heinz 1706 is depicted in SEQ ID NO: 4. It has a sequence identity of 99.8% to the protein of SEQ ID NO: 2, as it only contains one different amino acid. The last amino acid, amino acid 658, is Histidine (His) in cultivar Moneymaker (SEQ ID NO: 2) and Serine (Ser) in cultivar Heinz 1706. The gene encoding the Heinz 1706 protein can thus be considered an allelic variant of the gene found in the fresh market cultivar Moneymaker.

An “SlARF9 protein” (including “variants” thereof, such as proteins encoded by allelic variants of the gene or by orthologs of the gene) may be defined by their amino acid sequence identity to SEQ ID NO: 2 over the entire length, i.e. proteins having a sequence identity of at least about 55%, 60%, 70%, 80%, 90%, 95%, 98%, 99% or more to SEQ ID NO: 2 (as determined by pairwise alignment using Emboss “Needle”, Blossum 62 matrix, GAP opening penalty=10, GAP extension penalty=0.5) and having an in vivo function which is essentially similar to that of SlARF9.

Also included herein are loss-of-function mutants of the wild type SlARF9 proteins (or of variants thereof, as defined above) or reduced-function mutants of wild type SlARF9 proteins (or of variants thereof), as described elsewhere, and plants or plant parts comprising one or more nucleotides sequences encoding such mutants, whereby the mutant allele causes the plant to produce significantly larger fruits compared to plants comprising a nucleic acid sequence encoding a wild type SlARF9 protein. Significantly larger fruits are preferably fruits which weigh (on average) at least about 105%, preferably at least about 110%, 120%, 130% 140%, 150%, or more, of the average fresh fruit weight of the fruits of wild type plants. Optionally also the average fruit diameter is at least 105%, 110%, 115%, or more of the fruit diameter of wild type plants.

Optionally also the average number of cells in the pericarp tissue and/or the number of cell layers of the pericarp tissue are significantly higher than in the controls (e.g. fruits of wild type plants). Also the average cell size of pericarp cells, especially of mesocarp cells) is preferably significantly smaller than in the controls. Preferably the average number of pericarp cells (as measurable per unit area, see Examples) is at least about 105%, 110%, 120%, 130%, 140%, 150%, 160% or more of the control. Preferably the number of cell layers is at least 105%, 110%, 120% or more of the control. Preferably also the average size of pericarp cells (especially mesocarp cells) is at least about 95%, 92%, 90%, 85%, 75%, 72% or less, of the average cell size of the controls.

A “function which is essentially similar to the function of SlARF9” refers herein to the protein having a proven function in determining fruit size. Plants overexpressing SlARF9, or a variant thereof, in at least developing fruit tissue, produce (on average) significantly smaller fruits compared to controls (e.g. wild type plants or plants transformed with an empty vector). Vice versa, plants with reduced levels of fully functional (wild type) SlARF9 protein, or a variant thereof, in at least developing fruit tissue, produce (on average) significantly larger fruits compared to controls (e.g. wild type plants or plants transformed with an empty vector). As shown in the examples, SlARF9 overexpressing lines had an average fruit weight which was less than 75% of the wild type weight and an average fruit diameter which was less than 90% of the wild type. In contrast, SlARF9 silenced lines had an average fruit weight which was higher than 125% of the wild type weight and a diameter which was above 105% of the wild type fruit diameter.

Thus, the function of a (putative) SlARF9 protein can be tested using a variety of known methods, e.g. by comparing the phenotype of transformants constitutively expressing the protein being tested to the phenotype of SlARF9 over-expressing transformants of the same host species (and variety) (preferably comprising a chimeric SlARF9 encoding gene stably integrated into the host's genome), allowing a direct comparison of the functional effect on the phenotype of the transformants.

Similarly, transformants in which the SlARF9 gene (or variant) is silenced or down-regulated (e.g. mRNA of SlARF9 is significantly reduced at least in developing fruit tissue compared to wild type or control transformants) can be used to determine the function. A “significant reduction” of the mRNA of SlARF9 transcript refers to the target mRNA being present at a level of less than or equal to 90%, 80%, 70%, 60%, 50% 40%, 30%, 20% or less (10%, 5% or 0%) of the transcript level found in the wild type or control transformants (e.g. empty vector transformant). It is understood that in any transformation experiments a certain degree of variation in the phenotype of transformants is seen, normally due to position effects in the genome and/or due to copy number. A skilled person will know how to compare transformants to one another, e.g. by selecting single copy number events and analysing their phenotypes. Other methods of determining or confirming in vivo gene/protein function include the generation of knock-out mutants or reduced-function mutants (e.g. by TILLING) or transient expression studies. Promoter-reporter gene expression studies may also provide information as to the spatio-temporal expression pattern and the role of the protein.

The above methods can be used to test whether any putative SlARF9 gene, such as an allele from a wild relative of tomato or from a cultivated tomato plant or from a tomato breeding line or PI (plant introduction) line or from a different species (e.g. watermelon, or other fruit or vegetable species or from field crop species) is indeed a SlARF9 variant or ortholog, which can then be used to generate transgenic and/or non-transgenic plants producing (significantly) larger fruits compared to suitable controls, such as the wild type plant. It is understood that regarding transgenic plants, preferably plants having good agronomic characteristics are transformed and regenerated, i.e. cultivated plants (for example high yielding cultivars or breeding lines) and that the most suitable controls are empty vector transformants of the same line or a plurality of plants of the non-transformed line as such.

The sequences provided herein can be used to identify, generate and/or isolate other SlARF9 or slarf9 alleles from other tomato plants, wild relatives of tomato, or orthologs from other species. The sequences can, thus, also be used to generate and/or identify plants comprising one or more mutant slarf9 alleles in their genome, whereby the mutation causes significantly increased fruit size. Thus, in one embodiment, the use of an SlARF9 gene to identify and/or generate plants comprising one or more mutant slarf9 alleles, capable of conferring larger fruit size, is provided.

Preferably the plants according to the invention, which comprise one or more mutant slarf9 alleles (or variants), and which produce larger fruits, do not produce fewer fruits than the wild type plants. Thus, fruit number per plant is preferably not reduced. In one embodiment of the invention the plants according to the invention produce fruits at the end of the fruit-production season which are similar in size or larger than wild type fruits are in the main fruit production season. In tomato, fruit size of wild type plants is smaller at the end of the production season due to environmental conditions. This end-of-season effect is compensated by the plants according to the invention.

Other putative SlARF9 genes/proteins can be identified in silico, e.g. by identifying nucleic acid or protein sequences in existing nucleic acid or protein database (e.g. GENBANK, SWISSPROT, TrEMBL) and using standard sequence analysis software, such as sequence similarity search tools (BLASTN, BLASTP, BLASTX, TBLAST, FASTA, etc.). Putative amino acid sequences or nucleic acid sequences comprising or encoding an SlARF9 protein (as defined above) are selected, cloned or synthesized de novo and tested for in vivo functionality by e.g. overexpression and/or silencing in a plant host. It is noted that the designation SlARF9 is also used herein for proteins which are derived from species other than Solanum lycopersicum, i.e. the prefix Sl does herein not limit the protein as being from a particular species.

In one embodiment reduced-function or loss-of-function mutant SlARF9 proteins (including variants or orthologs as defined) are provided and plants and plant parts comprising one or more slarf9 alleles in their genome, which encode reduced-function or loss-of-function mutants, whereby the reduced-function or loss-of-function confers significantly enhanced fruit size when present in the plant genome.

Any type of mutation may lead to a reduction in function or loss of function of the encoded SlARF9 protein, e.g. insertion, deletion or replacement of one or more nucleotides in the cDNA (SEQ ID NO: 1, or variants) or in the corresponding genomic SlARF9 sequence (nucleotides 2005-5879 of SEQ ID NO: 3, nucleotides 1196-5869 of SEQ ID NO: 4 or variants of these), especially in any of the 14 exon sequences (see SEQ ID NO: 3 and SEQ ID NO: 4) and/or intron/exon boundaries of SlARF9 proteins (or variants thereof, including orthologs). In a preferred embodiment, a slarf9 nucleic acid sequence capable of conferring enhanced fruit size is provided, whereby the nucleic acid sequence encodes a reduced-function or loss-of-function SlARF9 protein due to one or more mutations in the region encoding the DNA binding domain (amino acid 74-236 of SEQ ID NO: 2, encoded by exons 3-8), the MR (amino acids 237-564 of SEQ ID NO: 2, encoded by exons 8-12, and within the MR amino acids 256-332 are particularly preferred, encoded by exons 8-10), the dimerization domain III (amino acids 565-602 of SEQ ID NO: 2, encoded by exons 12 and 13) and/or the dimerization domain IV (amino acids 609-651 of SEQ ID NO: 2, encoded by exons 13 and 14).

The effect of a mutation on protein function (reduced-function or loss-of function) can be predicted by SIFT analysis (Pauline C. Ng and Henikoff 2003, Nucleic Acid Research Vol. 31, pp 3812-3814) or determined by assessing the effect on fruit size (phenotyping).

The in vivo reduced-function or loss-of-function of such proteins can be tested as described above, by determining the effect this mutant allele has on significantly enhancing fruit size. Plants comprising a nucleic acid sequence encoding such mutant reduced-function or loss-of-function proteins and producing significantly larger fruits, can for example be generated using TILLING or identified using EcoTILLING, as described further below.

In one embodiment of the invention (cDNA or genomic) nucleic acid sequences encoding such mutant proteins comprise one or more non-sense and/or mis-sense mutations, e.g. transitions (replacement of purine with another purine (A [symbol] G) or pyrimidine with another pyrimidine (C [symbol] T)) or transversions (replacement of purine with pyrimidine, or vice versa (C/T [symbol] A/G). In one embodiment the non-sense and/or mis-sense mutation(s) is/are in the nucleotide sequence encoding any of the SlARF9 exons, more preferably in regions encoding protein domains mentioned above (the DNA binding domain, MR, Dimerization domain III and/or dimerization domain IV) or an essentially similar domain of a variant SlARF9 protein, i.e. in a domain comprising at least 80%, 90%, 95%, 98%, 99% amino acid identity to the domain of SEQ ID NO: 2).

In one embodiment a slarf9 nucleotide sequence comprising one or more non-sense and/or mis-sense mutations in the exon 2-, exon 3-, exon 4-, exon 5-, exon 6- and/or exon 7-encoding sequence are provided, as well as a plant comprising such a mutant allele and producing significantly larger fruits than plants comprising only wild type alleles (encoding functional SlARF9 protein).

In a specific embodiment of the invention plants and plant parts (fruits, seeds, etc.) comprising a mutant loss-of-function or reduced-function slarf9 allele are provided.

In one embodiment, the loss-of-function or reduced-function SlARF9 protein is a truncated protein, i.e. a protein fragment of any one of the SlARF9 proteins defined further above (including variants thereof). In general EMS (Ethyl methanesulfonate) induces substitutions of guanine/cytosine to adenin/thymine. In case of a glutamine (Gln or Q, encoded by the nucleotides CAA or CAG) or arginine (Arg or R, encoded by the nucleotides CGA) codon, a substitution of the cytosine for thymine can lead to the introduction of a stop codon in the reading frame (for example CAA/CAG/CGA to TAA/TAG/TGA) resulting in a truncated protein.

Also provided are nucleic acid sequences (genomic DNA, cDNA, RNA) encoding SlARF9 proteins, such as for example SlARF9 depicted in SEQ ID NO:2 or variants thereof as defined above (including any chimeric or hybrid proteins or mutated proteins or truncated proteins), or any SlARF9 protein from another species. Due to the degeneracy of the genetic code various nucleic acid sequences may encode the same amino acid sequence. Any nucleic acid sequence encoding an SlARF9 protein (as defined above, including variants thereof) is herein referred to as SlARF9. The nucleic acid sequences provided include naturally occurring, artificial or synthetic nucleic acid sequences. A nucleic acid sequences encoding SlARF9 is provided for in SEQ ID NO: 1 (cDNA) and 3 (genomic sequence from tomato cv Moneymaker, with nucleotides 2005-5879 being the protein-coding region with introns) and in SEQ ID NO: 4 (genomic sequence from tomato cv Heinz 1706, with nucleotides 1196-5869 being the protein-coding region with introns).

It is understood that when sequences are depicted in as DNA sequences while RNA is referred to, the actual base sequence of the RNA molecule is identical with the difference that thymine (T) is replace by uracil (U).

Also provided are nucleic acid sequences (genomic DNA, cDNA, RNA) encoding mutant SlARF9 proteins, i.e. reduced function or loss-of-function SlARF9 proteins, as described above, and plants and plant parts comprising such mutant sequences. For example, slarf9 nucleic acid sequences comprising one or more non-sense and/or mis-sense mutations in the wild type SlARF9 coding sequence, rendering the encoded protein non-functional or having a reduced function in vivo. Also sequences with other mutations are provided, such as splice-site mutants, i.e. mutations in the genomic slarf9 sequence leading to aberrant splicing of the pre-mRNA, and/or frame-shift mutations, and/or insertions (e.g. transposon insertions) and/or deletions of one or more nucleic acids.

Two genomic wild type SlARF9 sequences are provided herein, one from fresh-market tomato cultivar Moneymaker (SEQ ID NO: 3) and the other from processing cultivar Heinz 1706 (SEQ ID NO: 4). As mentioned, the coding region is identical, except that the last codon is different, encoding a Histidine in SEQ ID NO: 3 and a Serine in SEQ ID NO: 4. The coding DNA sequences (without introns) have a sequence identity of 99.9%. The genomic coding DNA, including the introns (nucleotides 2005-5879 of SEQ ID NO: 3 and nucleotides 1996-5869 of SEQ ID NO: 4) have a sequence identity of 99.8%, as there are some nucleotide differences in the intron sequences. The promoters of the genes (nucleotides 1-2004 of SEQ ID NO: 3 and 1-1995 of SEQ ID NO: 4) also have a high sequence identity of 98.7%.

Also included are variants and fragments of SlARF9 nucleic acid sequences, such as nucleic acid sequences hybridizing to SlARF9 nucleic acid sequences, e.g. to SEQ ID NO: 1 or 3 (nucleotides 2005-5879), under stringent hybridization conditions as defined. Variants of SlARF9 nucleic acid sequences also include nucleic acid sequences which have a sequence identity to SEQ ID NO: 1 or to SEQ ID NO: 3 (nucleotides 2005-5879) or to SEQ ID NO: 4 (nucleotides 1996-5869) of at least 60% or more, preferably at least 65%, 70%, 80%, 85%, 90%, 95%, 98%, 99% or more (as determined by Emboss “needle” using default parameters, i.e. gap creation penalty=10, gap extension penalty=0.5, scoring matrix nwsgapdna). The variants include also mutant slarf9 variants as described. It is clear that many methods can be used to identify, synthesise or isolate variants or fragments of SlARF9 nucleic acid sequences, such as nucleic acid hybridization, PCR technology, in silico analysis and nucleic acid synthesis, and the like. Variants of SEQ ID NO: 1, SEQ ID NO: 3 (nucleotides 2005-5879) or SEQ ID NO: 4 (nucleotides 1996-5869) may either encode wild type, functional SlARF9 proteins (e.g. alleles from other tomato varieties or breeding lines or wild accessions, or orthologs from other species than tomato), or they may encode reduced-function or loss-of function mutant alleles of any of these, as for example generated or identified by methods such as TILLING or EcoTILLING, or other methods.

Fragments include parts of any of the above SlARF9 nucleic acid sequences (or variants), which may for example be used as primers or probes or in gene silencing constructs or to detect mutant slarf9 alleles (e.g. primers used in TILLING, for example primers of SEQ ID NO: 15-22). Parts may be contiguous stretches of at least about 10, 15, 19, 20, 21, 22, 23, 24, 25, 50, 60, 100, 200, 300, 420, 450, 500, 600, 700, 800, 900, or more, nucleotides in length, of either the coding strand (sense strand) or the complementary strand (anti-sense strand). Also sense-antisense constructs of such fragments are included, which are capable of forming double stranded RNA (optionally with a spacer sequence in between the sense and the antisense fragment) when transcribed in a plant cell (see gene silencing). Also included are, therefore, fragments of SlARF9 nucleic acid sequences, whereby a fragment of at least about 10, 15, 20, 30, 40, 50, 60, 100, 150, 200 300, 400, 420, 450, 500, 600, 700, 800, 900 nucleotides in length comprises at least 50, 60, 70, 75%, more preferably at least 80, 90, 95, 98, 99% or more (100%) nucleic acid sequence identity to another fragment of a SlARF9 nucleic acid sequence of about the same length (as determined by pairwise alignment using Emboss “needle” using default parameters, i.e. gap creation penalty=10, gap extension penalty=0.5, scoring matrix nwsgapdna).

Primer pairs can be used for PCR amplification of SlARF9 or slarf9 transcripts (mRNA or corresponding cDNA or genomic DNA) from plant tissue DNA sample. Primer pairs can be used to detect and/or quantify SlARF9 or slarf9 expression (mRNA levels) in plant tissue, e.g. in tomato fruit tissue, e.g. or to determine whether endogenous SlARF9 mRNA levels are significantly reduced or whether a mutant slarf9 allele is present in the genome. Likewise specific or degenerate primers can be designed based on SEQ ID NO: 1 or SEQ ID NO: 3 and used to amplify/detect variants alleles of SlARF9 (e.g. mutant slarf9 alleles) from/in other tomato lines, from wild relatives of tomato or from other plant species.

Once plant tissue comprising a specific mutant slarf9 allele has been generated and/or identified (e.g. by TILLING or EcoTILLING), also primers or probes specific for the mutant allele can be designed and an assay can be developed which detects the presence and/or absence of the mutant allele in a plant or plant part (using allele specific detection assays). Molecular marker assays for detection and/or transfer (e.g. by MAS, marker assisted selection) of the mutant allele can be developed. E.g. a SNP detection assay or a CAPS marker can be developed which detects the presence of mutant slarf9 nucleic acid sequence in DNA of plants and/or which can be used for transfer of the allele into other plants.

In one embodiment mutant slarf9 nucleic acid sequences are provided, whereby the slarf9 nucleic acid sequence comprises one or more mutations leading to either a loss-of-function mutant of the SlARF9 protein or a reduced-function mutant of the SlARF9 protein. This aspect of the invention is described in more detail elsewhere herein.

Plants can also be identified or generated (e.g. by homologous recombination, or by insertion, deletion or replacement of one or more nucleotides, etc.) which have one or more mutations in the SlARF9 regulatory region(s), e.g. the promoter, whereby SlARF9 gene expression, i.e. mRNA levels (of SEQ ID NO: 1 or variants) is/are significantly reduced in the plant compared to the wild type and whereby the plant produces significantly larger fruits than the wild type plant comprising the wild type regulatory region (e.g. promoter).

The promoter region of SlARF9 is depicted in nucleotides 1-2004 of SEQ ID NO: 3 and nucleotides 1-1995 in SEQ ID NO: 4. These two promoter sequences have 98.7% sequence identity. The wild type tomato SlARF9 promoter is active during early fruit development stages (in pericarp, ovules, placenta of pollinated ovaries), in axillary meristems, root tips and lateral root primordial. SEQ ID NO: 3 and 4 both comprise two auxin-response elements (AuxRE) in the promoter region, at position 612-617 (TGTCNC) and 1224-1229 (TGTCTN) of SEQ ID NO: 3 and at position 612-617 (TGTCNC) and 1229-1234 ((TGTCTN) of SEQ ID NO: 4. Further, SEQ ID NO: 3 comprises four NTBBF1ARROLB-elements (ACTTTA, at positions 888-893, 1541-1546, 1824-1829 and 1831-1836), while SEQ ID NO: 4 comprises only three such elements (at position 888-839, 1815-1820 and 1822-1827). These cis-acting regulatory elements may confer transcriptional regulation by other ARFs and/or by Dof-like proteins. Mutations in these elements may reduce the production of SlARF9 transcript, leading to plants with larger fruits. Thus in one embodiment plants, plant parts or tissues comprising one or more mutations in the endogenous (wild type) SlARF9 promoter region, especially in one or more AuxRE and/or NTBBF1ARROLB-elements, are provided, whereby these plants produce significantly larger fruits. Such plants can for example be generated by TILLING. Specifically mutations (transitions or transversions) in the G in the AuxRE and/or the C in the NTBBF1ARROLB element(s) are encompassed herein. Plants comprising mutations in the SlARF9 promoter, whereby the promoter activity is significantly reduced, at least during early fruit development, and whereby the plant produces significantly larger fruits can be identified e.g. by TILLING or by other known methods.

An “SlARF9 promoter” according to the invention is a promoter comprising at least 80%, preferably at least 90%, 95%, 98%, 99% or 100% nucleic acid sequence identity to nucleotides 1-2004 of SEQ ID NO: 3 or to nucleotides 1-1995 of SEQ ID NO: 4 (using pair wise alignment program Needle with default parameters). In vivo, in its natural environment, such a promoter drives expression of a SlARF9 nucleic acid sequence, or variant (e.g. a wild type SlARF9 or mutant slarf9 gene) according to the invention. Mutant SlARF9 promoters (and plants comprising these) are encompassed herein.

Also encompassed herein are chimeric genes and vectors comprising an SlARF9 promoter and transgenic plants comprising an SlARF9 promoter, operably linked to a protein encoding nucleic acid or a gene silencing construct (sense and/or antisense sequence). The promoter can be used to express chimeric genes in developing fruits. Active fragments of at least about 2000, 1700, 1600, 1500, 1200, 100, 800, 600, 500, 400, 300 nucleotides (obtained e.g. by making promoter deletions at the 5′ end) of the SlARF9 promoter may also be suitable for fruit-preferred expression.

The SlARF9 nucleic acid sequence described above, or fragments thereof, particularly DNA sequences encoding the SlARF9 proteins of this invention (or variants of these) can be inserted into expression vectors (in co-suppression approaches) or into gene silencing vectors to generate plants with larger fruits.

In one embodiment of the invention SlARF9 gene expression is downregulated in a host cell, plant or specific tissue(s), by e.g. RNAi approaches, as described elsewhere.

In another embodiment plants comprising one or more endogenous mutant slarf9 alleles are provided, whereby the mutation(s) confer larger fruit size on the plant compared to plants lacking the mutant allele(s). Mutant alleles are preferably generated by mutagenesis of the plant or seed and by identifying those plants or seeds which comprise one or more mutations in the target SlARF9 allele(s) and whereby the mutation results in reduction or abolishment of mRNA transcription and/or translation (so that no or reduced SlARF9 protein is produced), or in transcription of a mutant slarf9 allele, that is translated into a reduced-function or loss-of-function SlARF9 protein. The reduction or abolishment of functional, wild type SlARF9 protein, at least in fruit tissue (preferably at least during the early fruit development stage), confers the ability to produce significantly larger fruits on the plant or seed. In one embodiment of the invention the plant comprises two endogenous mutant slarf9 alleles, i.e. is homozygous for slarf9. Such a plant can be generated by selfing a plant comprising a single mutant slarf9 allele. Also fruits and seeds comprising at least one, or two, mutant slarf9 alleles in their genome are provided, whereby the fruits are significantly larger than those of plants comprising wild type SlARF9 alleles (encoding functional SlARF9 protein).

In another embodiment of the invention PCR primers and/or probes and kits for detecting the SlARF9 or slarf9 DNA sequences are provided. Degenerate or specific PCR primer pairs to amplify SlARF9 or slarf9 DNA from samples can be synthesized based on SEQ ID NO: 1 or SEQ ID NO: 3 or 4 as known in the art (see Dieffenbach and Dveksler (1995) PCR Primer: A Laboratory Manual, Cold Spring Harbor Laboratory Press, and McPherson at al. (2000) PCR-Basics: From Background to Bench, First Edition, Springer Verlag, Germany). Likewise, DNA fragments of SEQ ID NO: 1 or SEQ ID NO: 3 (or variants thereof) can be used as hybridization probes. A SlARF9 or slarf9 detection kit may comprise either SlARF9 and/or slarf9 specific primers and/or SlARF9 and/or slarf9 specific probes, and an associated protocol to use the primers or probe to detect SlARF9 and/or slarf9 DNA in a sample. Such a detection kit may, for example, be used to determine, whether a plant has been transformed with an SlARF9 gene (or part thereof) of the invention or whether a plant comprises one or more mutant slarf9 alleles. Because of the degeneracy of the genetic code, some amino acid codons can be replaced by others without changing the amino acid sequence of the protein.

In another embodiment antibodies that bind specifically to a SlARF9 protein, or mutant SlARF9 protein, according to the invention are provided. In particular monoclonal or polyclonal antibodies that bind to SlARF9, or to fragments or variants thereof (e.g. mutant proteins), are encompassed herein. An antibody can be prepared by using a SlARF9 protein according to the invention as an antigen in an animal using methods known in the art, as e.g. described in Harlow and Lane “Using Antibodies: A laboratory manual” (New York: Cold Spring Harbor Press 1998) and in Liddell and Cryer “A Practical Guide to Monoclonal Antibodies” (Wiley and Sons, 1991). The antibodies can subsequently be used to isolate, identify, characterize or purify the SlARF9 protein to which it binds, for example to detect the SlARF9 protein in a sample, allowing the formation of an immunocomplex and detecting the presence of the immunocomplex by e.g. ELISA (enzyme linked immunoassay) or immunoblot analysis. Also provided are immunological kits, useful for detecting the SlARF9 proteins, protein fragments or epitopes in a sample provided. Samples may be cells, cell supernatants, cell suspensions, tissues, etc. Such a kit comprises at least an antibody that binds to a SlARF9 protein and one or more immunodetection reagents. The antibodies can also be used to isolate/identify other SlARF9 proteins, for example by ELISA or Western blotting.

It is clear that many methods can be used to identify, synthesise or isolate variants or fragments of SlARF9 or slarf9 nucleic acid sequences, such as nucleic acid hybridization, PCR technology, in silico analysis and nucleic acid synthesis, and the like. Thus, an SlARF9-protein encoding nucleic acid sequence may be a sequence which is chemically synthesized or which is cloned from any plant species.

Transgenic Plants with Larger Fruits

Transgenic plants, seeds and plant parts are provided in which SlARF9 is silenced, preferably at least in fruit tissue, and which produce significantly larger fruits compared to wild type (non-transgenic) control plants or other control plants (e.g. empty vector transformants) as a result of the SlARF9 gene silencing.

In one embodiment of the invention a homologous or heterologous nucleic acid sequence is used to silence the endogenous SlARF9 gene(s) of the host species to be transformed. For example, a potato SlARF9 gene (or variant or fragment thereof) may be used to silence SlARF9 gene expression in transgenic tomato or aubergine or watermelon plants. Alternatively, homologous SlARF9 nucleic acid sequences may be used. For example a sequence originating from a particular plant species (e.g. from tomato) is reintroduced into said species (tomato). Thus, in one embodiment, the SlARF9 DNA corresponds to, or is a modification/variant of, the endogenous SlARF9 DNA of the species which is used as host species in transformation. Thus, a tomato SlARF9 cDNA or genomic DNA (or a variant or fragment thereof) is preferably used to transform tomato plants. In addition (for regulatory approval and public acceptance reasons) the homologous or heterologous nucleic acid sequence may be operably linked to a transcription regulatory sequence, especially a promoter, which also originates from a plant species or even from the same plant which is to be transformed. For example an SlARF9 promoter as defined above may be used in tomato.

To generate plants comprising a chimeric gene, which upon expression results in silencing of the expression of an endogenous SlARF9 gene or gene family, methods known in the art can be used.

“Gene silencing” refers to the down-regulation or complete inhibition of gene expression of one or more target genes, e.g. SlARF9 genes, in a host cell or tissue. It is understood that in any transformation experiments a certain degree of variation in the phenotype of transformants is seen, normally due to position effects in the genome and/or due to copy number. Generally, “weak” and “strong” gene silencing plants are distinguished herein (all of which are embodiments of the invention), wherein “weak” gene silencing (RNAi) events refer to plants or plant parts wherein the endogenous target gene expression is reduced by about 15, 20 or 30% compared to the control tissue and “strong” gene silencing (RNAi) events refer to plants or plant parts wherein the endogenous target gene expression is reduced by at least about 50, 60, 70, 80, 90% or more compared to the control tissue (e.g. wild type). Silencing can be quantified by, for example, quantifying the transcript level of the target gene (e.g. using quantitative RT-PCR) and/or by determining and optionally quantifying the enzymatic activity of the target protein and/or by assessing and optionally quantifying resulting phenotype (larger fruit size).

Without limiting the scope of the invention, plants having an optimal silencing level can be selected, so that resulting plants have significantly larger fruits under the climatic conditions to which they are exposed during fruit development, while having minimal negative side-effects, such as reduced yield, etc. compared to controls. Preferably yield is significantly is increased in the SlARF9 silenced plants.

The use of inhibitory RNA to reduce or abolish gene expression is well established in the art and is the subject of several reviews (e.g Baulcombe 1996, Plant Cell 8(2):179-188; Depicker and Van Montagu, 1997, Curr Opin Cell Biol. 9(3): 373-82). There are a number of technologies available to achieve gene silencing in plants, such as chimeric genes which produce antisense RNA of all or part of the target gene (see e.g. EP 0140308 B1, EP 0240208 B1 and EP 0223399 B1), or which produce sense RNA of the target gene (also referred to as “co-suppression”), see EP 0465572 B1.

The most successful approach so far has however been the production of both sense and antisense RNA of the target gene (“inverted repeats”), which forms double stranded RNA (dsRNA) or a stem-loop structure (hairpin RNA, hpRNA) in the cell and silences the target gene(s) upon transcription from an upstream promoter. Methods and vectors for dsRNA and hpRNA production and gene silencing have been described in EP 1068311, EP 983370 A1, EP 1042462 A1, EP 1071762 A1 and EP 1080208 A1.

A chimeric gene for plant transformation may, therefore, comprise a transcription regulatory region which is active in plant cells operably linked to a sense and/or antisense DNA fragment (or a complete nucleic acid sequence) of, or complementary or substantially similar to, a SLARF9 target gene or gene family.

Generally short (sense and anti-sense) stretches of the target gene sequence, such as 17, 18, 19, 20, 21, 22, 23, 24, 25 or 26 nucleotides of coding and/or non-coding sequence of the target gene are sufficient. Longer sequences can also be used, such as at least about 50, 100, 200, 250, 300, 400, 420, 450, 500, 1000 or more nucleotides. Even DNA corresponding to, or being complementary to, the complete transcript RNA or mRNA may be used to make a sense and/or antisense construct. Preferably, the sense and antisense fragments/sequences are separated by a spacer sequence, such as an intron, which forms a loop (or hairpin) upon dsRNA formation.

In principle, any SlARF9 gene or gene family can be targeted. For example, one or several specific SlARF9 alleles may be silenced by choosing a nucleic acid region of their primary or mRNA transcripts specific for these alleles (see Byzova et al. Plant 2004 218: 379-387 for allele specific silencing in an organ specific manner). Similarly, a whole gene family may be targeted for silencing by choosing one or more conserved regions of the transcripts for making the silencing construct. As mentioned above, the DNA region used in sense and/or antisense orientation does not need to be part of the coding region, but may also correspond to, or be complementary to, parts of the primary transcript (comprising a 5′ and 3′ untranslated sequence and introns; the primary transcript of SlARF9 from tomato cv Moneymaker is depicted in SEQ ID NO: 3, from nucleotide 1551 to 6323, with the coding region being present from nucleotide 2005 to 5879) or to parts of the mRNA transcript (where any introns have been removed and a polyA tail has been added). It is understood that in a DNA sequence which corresponds to an RNA sequence the U is replaced by a T. It is also noted that in a chimeric gene which transcribes a dsRNA or hpRNA targeting capable of silencing SlARF9 gene expression upon transcription in a host cell, the sense and antisense regions need not be of equal length and one region may be longer than the other.

Thus, for example SEQ ID NO: 1 or variants thereof as described above, or fragments of any of these, or the genomic sequence or primary transcript sequence of SEQ ID NO: 3 or 4, may be used to make a SlARF9 gene silencing gene and vector and a transgenic plant in which one or more SlARF9 genes are silenced in all or some tissues or organs (preferably at least in the developing fruits), or upon induction (see e.g. Wielopolska et al. Plant Biotechnol J. 2005 6:583-90). An example of a gene silencing vector is given in the Examples, whereby an inverted repeat of a 420 bp fragment of the Middle Region (MR) encoding part of SEQ ID NO: 1 was used to silence endogenous SlARF9 in tomato, using constitutive expression under the control of the CaMV 35S promoter.

A convenient way of generating hairpin constructs is to use generic vectors such as pHANNIBAL, pHELLSGATE, pSTARGATE vectors based on the Gateway® technology (see Wesley et al. 2004, Methods Mol Biol. 265:117-30; Wesley et al. 2003, Methods Mol Biol. 236:273-86 and Helliwell & Waterhouse 2003, Methods 30(4):289-95.), incorporated herein by reference. See also http://www.pi.csiro.au/rnai/ for other gene silencing vectors, such as inducible silencing vectors and vectors for silencing of multiple target genes and for the program MatchPoint which can be used to find the best sequence to use for silencing the target gene.

By choosing conserved nucleic acid sequences all SlARF9 gene family members in a host plant can be silenced. The silencing of all family members of a host plant is an embodiment.

In one embodiment the promoter, which is operably linked to the sense and/or antisense nucleic acid sequence (to make a chimeric silencing/RNAi gene) is selected from a constitutive promoter, an inducible promoter (e.g. chemically inducible, etc.), a hormone inducible promoter (e.g. auxin inducible) a fruit specific promoter or developmentally regulated promoter (e.g. active during fruit development). Also, the promoter of a SlARF9 gene itself may be used for silencing approaches, i.e. a SlARF9 promoter as defined above. Optionally a 3′ UTR may be operably linked to the 3′ end of the chimeric gene, so that the operably linked DNA elements include promoter-SlARF9 RNAi gene-3′UTR.

Preferred constitutive promoters include: the strong constitutive 35S promoters or enhanced 35S promoters (the “35S promoters”) of the cauliflower mosaic virus (CaMV) of isolates CM 1841 (Gardner et al., 1981, Nucleic Acids Research 9, 2871-2887), CabbB-S (Franck et al., 1980, Cell 21, 285-294) and CabbB-JI (Hull and Howell, 1987, Virology 86,482-493); the 35S promoter described by Odell et al. (1985, Nature 313, 810-812) or in U.S. Pat. No. 5,164,316, promoters from the ubiquitin family (e.g. the maize ubiquitin promoter of Christensen et al., 1992, Plant Mol. Biol. 18,675-689, EP 0 342 926, see also Cornejo et al. 1993, Plant Mol. Biol. 23, 567-581), the gos2 promoter (de Pater et al., 1992 Plant J. 2, 834-844), the emu promoter (Last et al., 1990, Theor. Appl. Genet. 81,581-588), Arabidopsis actin promoters such as the promoter described by An et al. (1996, Plant J. 10, 107.), rice actin promoters such as the promoter described by Zhang et al. (1991, The Plant Cell 3, 1155-1165) and the promoter described in U.S. Pat. No. 5,641,876 or the rice actin 2 promoter as described in WO070067; promoters of the Cassava vein mosaic virus (WO 97/48819, Verdaguer et al. 1998, Plant Mol. Biol. 37,1055-1067), the pPLEX series of promoters from Subterranean Clover Stunt Virus (WO 96/06932, particularly the S7 promoter), a alcohol dehydrogenase promoter, e.g., pAdh1S (GenBank accession numbers X04049, X00581), and the TR1′ promoter and the TR2′ promoter (the “TR1′ promoter” and “TR2′ promoter”, respectively) which drive the expression of the l′ and 2′ genes, respectively, of the T-DNA (Velten et al., 1984, EMBO J 3, 2723-2730), the Figwort Mosaic Virus promoter described in U.S. Pat. No. 6,051,753 and in EP426641, histone gene promoters, such as the Ph4a748 promoter from Arabidopsis (PMB 8: 179-191), or others.

Alternatively, a promoter can be utilized which is not constitutive but rather is specific for one or more tissues or organs of the plant (tissue preferred/tissue specific, including developmentally regulated promoters). For example, a promoter active in fruit tissue and/or during fruit development. E.g. the SlARF9 promoter, or an active fragment thereof, may be used. Alternatively, the TPTP-F1 promoter, which is ovary and young fruit specific, may be used (Carmi et al. 200, supra), or others.

The skilled person can easily test various promoters for their specificity and suitability in the methods according to the invention. In addition, the specificity of promoters may be modified by deleting, adding or replacing parts of the promoter sequence. Such modified promoters can be operably linked to reporter genes in order to test their spatio-temporal activity in transgenic plants.

Another alternative is to use a promoter whose expression is inducible. Examples of inducible promoters are chemical inducible promoters, such as dexamethasone as described by Aoyama and Chua (1997, Plant Journal 11: 605-612) and in U.S. Pat. No. 6,063,985 or by tetracycline (TOPFREE or TOP 10 promoter, see Gatz, 1997, Annu Rev Plant Physiol Plant Mol Biol. 48: 89-108 and Love et al. 2000, Plant J. 21: 579-88).

Optionally, the promoter-SlARF9 RNAi gene may further comprise a 3′end transcription regulation signals (“3′end” or “3′ UTR”) (i.e. transcript formation and polyadenylation signals). Polyadenylation and transcript formation signals include those of, the nopaline synthase gene (“3′ nos”) (Depicker et al., 1982 J. Molec. Appl. Genetics 1, 561-573.), the octopine synthase gene (“3′ ocs”) (Gielen et al., 1984, EMBO J 3, 835-845) and the T-DNA gene 7 (“3′ gene 7”) (Velten and Schell, 1985, Nucleic Acids Research 13, 6981-6998), which act as 3′-untranslated DNA sequences in transformed plant cells, and others. Also the 3′end of the SlARF9 gene may be used, i.e. a sequence comprising or consisting of nucleotides 5880 to 6323 of SEQ ID NO: 3.

The chimeric SlARF9 silencing gene (i.e. the promoter operably linked to a nucleic acid sequence which upon transcription in a plant cell is capable of silencing the endogenous SlARF9 gene expression) can be stably inserted in a conventional manner into the nuclear genome of a single plant cell, and the so-transformed plant cell can be used in a conventional manner to produce a transformed plant that has an altered phenotype due to SlARF91 silencing in certain cells at a certain time. In this regard, a T-DNA vector, comprising a promoter operably linked to a sense and/or antisense SlARF9 sequence (and optionally a 3′UTR), may be introduced into Agrobacterium tumefaciens and used to transform the plant cell, and thereafter, a transformed plant can be regenerated from the transformed plant cell using the procedures described, for example, in EP 0 116 718, EP 0 270 822, PCT publication WO84/02913 and published European Patent application EP 0 242 246 and in Gould et al. (1991, Plant Physiol. 95,426-434). The construction of a T-DNA vector for Agrobacterium mediated plant transformation is well known in the art. The T-DNA vector may be either a binary vector as described in EP 0 120 561 and EP 0 120 515 or a co-integrate vector which can integrate into the Agrobacterium Ti-plasmid by homologous recombination, as described in EP 0 116 718.

Preferred T-DNA vectors each contain a promoter operably linked to SlARF9 silencing gene between T-DNA border sequences, or at least located to the left of the right border sequence. Border sequences are described in Gielen et al. (1984, EMBO J 3,835-845). Of course, other types of vectors can be used to transform the plant cell, using procedures such as direct gene transfer (as described, for example in EP 0 223 247), pollen mediated transformation (as described, for example in EP 0 270 356 and WO85/01856), protoplast transformation as, for example, described in U.S. Pat. No. 4,684,611, plant RNA virus-mediated transformation (as described, for example in EP 0 067 553 and U.S. Pat. No. 4,407,956), liposome-mediated transformation (as described, for example in U.S. Pat. No. 4,536,475), and other methods such as those described methods for transforming certain lines of corn (e.g., U.S. Pat. No. 6,140,553; Fromm et al., 1990, Bio/Technology 8, 833-839; Gordon-Kamm et al., 1990, The Plant Cell 2, 603-618) and rice (Shimamoto et al., 1989, Nature 338, 274-276; Datta et al. 1990, Bio/Technology 8, 736-740) and the method for transforming monocots generally (PCT publication WO92/09696). For cotton transformation see also WO 00/71733, and for rice transformation see also the methods described in WO92/09696, WO94/00977 and WO95/06722. For sorghum transformation see e.g. Jeoung J M et al. 2002, Hereditas 137: 20-8 or Zhao Z Y et al. 2000, Plant Mol Biol. 44:789-98). For tomato or tobacco transformation see also An G. et al., 1986, Plant Physiol. 81: 301-305; Horsch R. B. et al., 1988, In: Plant Molecular Biology Manual A5, Dordrecht, Netherlands, Kluwer Academic Publishers. pp 1-9; Koornneef M. et al., 1986, In: Nevins D. J. and R. A. Jones, eds. Tomato Biotechnology, New York, N.Y., USA, Alan R. Liss, Inc. pp 169-178). For potato transformation see e.g. Sherman and Bevan (1988, Plant Cell Rep. 7: 13-16). Tomato transformation and regeneration can also be carried out according to De Jong et al. (2008) Plant Journal 57:160-170 and Sun et al. (2006) Plant Cell Physiol. 47: 426-431.

Likewise, selection and regeneration of transformed plants from transformed cells is well known in the art. Obviously, for different species and even for different varieties or cultivars of a single species, protocols are specifically adapted for regenerating transformants at high frequency.

Besides transformation of the nuclear genome, also transformation of the plastid genome, preferably chloroplast genome, is included in the invention. One advantage of plastid genome transformation is that the risk of spread of the transgene(s) can be reduced. Plastid genome transformation can be carried out as known in the art, see e.g. Sidorov V A et al. 1999, Plant J. 19: 209-216 or Lutz K A et al. 2004, Plant J. 37(6):906-13.

Any plant may be a suitable host, such as monocotyledonous plants or dicotyledonous plants, but most preferably plants which would benefit from producing larger fruits, such as but not limited to: tomato, pepper, cucumber, aubergine, melon, watermelon, squash, pumpkin, grape, and many others, such as corn, wheat, rice, sorghum, sunflower, fruit trees, strawberries, citrus fruits, bean, pea, soybean, etc. Basically, any flowering plant species, which produces edible fruits (in the botanical sense) from ovaries is encompassed herein as host. Particularly preferred are fleshy fruit species (producing fruits with a fleshy pericarp).

Preferred hosts are of the family Solanaceae, such as species of the genus Solanum, e.g. tomato (S. lycopersicum), tree tomato (S. betaceum, syn. Cyphomandra betaceae) and other Solanum species, such as aubergine/eggplant (Solanum melongena), pepino (S. muricatum), cocona (S. sessiliflorum) and naranjilla (S. quitoense). The family Solanaceae also includes peppers (Capsicum annuum, Capsicum frutescens).

In a preferred embodiment the host is of the family Solanaceae or Cucurbitaceae. In a more preferred embodiment the host is of the genus Solanum. In an even more preferred embodiment the host is of the species S. lycopersicum. Preferably, the host is a cultivated tomato of the species S. lycopersicum, i.e. a line or variety yielding high yields, such as fruit of at least 50 g average fresh weight or more, e.g. at least about 80 g, 90 g, 100 g, 200 g, 300 g, or even up to 600 g (beef tomato types). Also small types, such as cherry or cocktail tomato are encompassed, as are full-flesh tomatoes such as the Nunhems variety Intense, e.g. lacking gel in the seed cavities. The host tomato plant may be determinate or indeterminate, of various fruit sizes and shapes, such as Roma type, cluster type, round. It may be a processing type tomato or a fresh market type. Also both open pollinated and hybrids are encompassed herein. In one embodiment the tomato plant is an F1 hybrid plant, grown from an F1 hybrid seed. To make F1 hybrid seeds of a transgenic plant according to the invention, two inbred parent lines may be made, each comprising a copy of the transgene in their genomes. When these plants are cross-fertilized, the F1 seeds are collected, which produce transgenic F1 hybrid plants with high yield and large fruits due to the transgene.

The embodiments described herein for ‘host’ plants also apply to non-transgenic mutant plants described elsewehere herein, whereby instead of a transgene a mutant slarf9 allele is present endogenously in the genome.

Other suitable hosts are other vegetable species and various species bearing fleshy fruits (grapes, peaches, plums, strawberry, mango, papaya, etc.). Also Cucurbitaceae, such as melon (Citrullus lanatus, Cucumis melo) and cucumber (Cucumis sativus) and squashes and marrows (Cucurbita) are suitable hosts. Likewise Rosaceae are suitable hosts, such as apple, pear, plum, etc.

Also field crops with larger (SlARF9 silencing or mutating slarf9) or smaller (SlARF9 overexpression) fruits (in the botanical sense) are provided according to the invention. For example maize/corn (Zea species, e.g. Z. mays, Z. diploperennis (chapule), Zea luxurians (Guatemalan teosinte), Zea mays subsp. huehuetenangensis (San Antonio Huista teosinte), Z. mays subsp. mexicana (Mexican teosinte), Z. mays subsp. parviglumis (Balsas teosinte), Z. perennis (perennial teosinte) and Z. ramosa), wheat (Triticum species), barley (e.g. Hordeum vulgare), oat (e.g. Avena sativa), sorghum (Sorghum bicolor), rye (Secale cereale), soybean (Glycine spp, e.g. G. max), cotton (Gossypium species, e.g. G. hirsutum, G. barbadense), Brassica spp. (e.g. B. napus, B. juncea, B. oleracea, B. rapa, etc), rice (Oryza species, e.g. O. sativa indica cultivar-group or japonica cultivar-group), pearl millet (Pennisetum spp. e.g. P. glaucum).

Basically, any crop plant species is suitable. A crop plant or cultivated plant refers herein to a plant species which is cultivated and bred by humans and excludes weeds such as Arabidopsis thaliana, or wild relatives, such as the tomato relatives and others (although mutant slarf9 alleles may be derived from such plants and transferred into cultivated plants by breeding methods, see further down). A crop plant may be cultivated for food or feed purposes (e.g. vegetable crops or field crops) or for ornamental purposes. A crop plant as defined herein also includes plants from which non-food products are harvested, such as oil for fuel, plastic polymers, pharmaceutical products, cork, fibers and the like.

Thus, in one embodiment of the invention transgenic plants comprising a transcription regulatory element (especially a promoter as described above) operably linked to nucleic acid molecule which upon transcription is capable of silencing the endogenous SlARF9 gene expression in the host cells.

The construction of chimeric genes and vectors for, preferably stable, introduction of SlARF9 silencing gene into the genome of host cells is generally known in the art. To generate a chimeric gene the sense and/or antisense SlARF9 sequence is operably linked to a promoter sequence, suitable for expression in the host cells, using standard molecular biology techniques. The promoter sequence may already be present in a vector so that the nucleic sequence is simply inserted into the vector downstream of the promoter sequence. The vector is then used to transform the host cells and the chimeric gene is inserted in the nuclear genome or into the plastid, mitochondrial or chloroplast genome and expressed there using a suitable promoter (e.g., Mc Bride et al., 1995 Bio/Technology 13, 362; U.S. Pat. No. 5,693,507).

The resulting transformed plant can be used in a conventional plant breeding scheme to produce more transformed plants with the same characteristics or to introduce the gene part into other varieties of the same or related plant species. An “elite event” can be selected, which is a transformation event having the transgene inserted in a particular location in the genome, which results in good expression of the desired phenotype (e.g. optimal silencing and large fruit size).

The transgenic plants, or parts thereof, in which SlARF9 is silenced, have significantly larger fruits, preferably with significantly more cells and/or significantly more cell layers and/or significantly smaller cells in the pericarp tissue. Significantly larger fruits (as described above) is used herein to refer to an enhanced average fruit weight and/or (optionally) fruit diameter and/or fruit volume compared to controls. Fruit weight can for example be compared at the end of the fruit growth phase, when the fruit has reached the final size. Fruit diameter can also be easily measured in round fruits, but is more difficult to compare in other shapes. Fruit volume can easily be determined by e.g. measuring the volume of liquid (e.g. water) in a container displaced by the fruits or other methods.

It is understood that when mutant plants are analyzed for their phenotype, the control plants are preferably near isogenic lines of the mutant, which comprise the wild type allele(s).

Ultimately, field trials may be used to show that transformants (or mutant plants described further down) produce significantly larger fruits compared to wild type plants.

As already mentioned, transformants having an optimal silencing level can be selected by e.g. analysing copy number (Southern blot analysis), mRNA transcript levels (e.g. RT-PCR using SlARF9 primer pairs) or by analysing the presence and/or level of SlARF9 protein in various tissues (e.g. SDS-PAGE; ELISA assays, etc). Optimal transgenic events are then used for further crossing/backcrossing/selfing until a high performing elite event with a stable transgene is obtained.

Transformants expressing one or more SlARF9 genes (or silencing-genes) according to the invention may also comprise other transgenes, such as genes conferring drought tolerance or conferring tolerance to biotic or abiotic stresses, herbicide tolerance, etc. To obtain such plants with “stacked” transgenes, other transgenes may either be introgressed into the SlARF9 transformants, or the transformants may be transformed subsequently with one or more other genes, or alternatively several chimeric genes may be used to transform a plant line or variety. For example, several chimeric genes may be present on a single vector, or may be present on different vectors which are co-transformed. Also mini-chromosomes comprising several chimeric genes may be introduced into a plant (see e.g. Yu et al. 2007, PNAS 104: 8924-9 and Houben and Schubert 2007, Plant Cell 19: 2323-2327).

Whole plants, seeds, cells, tissues and progeny (such as F1, F2 seeds/plants, etc) of any of the transformed plants described above are encompassed herein and can be identified by the presence of the transgene in the DNA, for example by PCR analysis. Also “event specific” PCR diagnostic methods can be developed, where the PCR primers are based on the plant DNA flanking the inserted chimeric gene, see U.S. Pat. No. 6,563,026. Similarly, event specific AFLP fingerprints or RFLP fingerprints may be developed which identify the transgenic plant or any plant, seed, tissue or cells derived there from.

It is understood that the transgenic plants according to the invention preferably do not show non-desired phenotypes, such as reduced fruit quality, fewer fruits per plant, enhanced susceptibility to diseases or undesired architectural changes (dwarfing, deformations) etc. and that, if such phenotypes are seen in the primary transformants, these can be removed by normal breeding and selection methods (crossing/backcrossing/selfing, etc.). Any of the transgenic plants described herein may be homozygous or hemizygous for the transgene.

Non-Transgenic Plants Producing Larger Fruits and Methods for Making these

It is also an embodiment of the invention to use non-transgenic methods, e.g. target mutant generation and identification systems such as TILLING (Targeting Induced Local Lesions IN Genomics; McCallum et al., 2000, Nat Biotech 18:455, and McCallum et al. 2000, Plant Physiol. 123, 439-442, Henikoff et al. 2004, Plant Physiol. 135: 630-636 incorporated herein by reference) and selection to generate plant lines which comprise at least one mutation in an endogenous SlARF9 allele and whereby the plants comprising the mutant slarf9 allele in heterozygous or homozygous form produce significantly larger fruits compared to plants lacking the mutant allele (having wild type allele(s) at the SlARF9 locus). Thus, in one embodiment of the invention plants comprising one or more mutant slarf9 alleles in the genome and producing larger fruits compared to plants lacking said mutant allele(s), but comprising wild type alleles instead, are provided herein, as well as plant parts (e.g. harvested fruit, harvested leaves, etc.), seeds, clonal propagations of such plants, progeny of such plants comprising the mutant allele.

The term “significantly larger fruits refers herein to the fruit size being (on average) significantly larger compared to suitable control plants (e.g. wild type plants), i.e. the average equatorial diameter and/or average volume and/or average fresh fruit weight being significantly higher than in the controls. The fruit size is preferably determined at the end of the fruit growth phase or later on (i.e. when the fruit has reached its final size). For example in tomato, fruit size of at least about 5, 8, 10, 15, 20 or more fruits per plant may be determined by measuring the average fruit fresh weight at the end of the growth phase (e.g. at breaker stage) and/or by measuring the average equatorial diameter and comparing the values to the controls (fruits from plants comprising wild type SlARF9 alleles in their genome). Plants producing significantly larger fruits produce for example fruits which have an average weight of at least about 105, 110, 115, 120, 130, 140, 150% or more of the control fruits. Optionally or alternatively average equatorial diameter may be at least about 105, 110, 115, 120% or more of the equatorial diameter of the control fruits.

Optionally, also the number of cells and/or the number of cell layers in the pericarp tissue is significantly higher, and/or the cell size is significantly smaller compared to controls (e.g. wild type fruit), as described elsewhere.

Without limiting the invention, it is thought that fruits of plants comprising mutant slarf9 alleles encoding non-function or reduced function SlARF9 protein are significantly larger and have more cells due to increased cell division during fruit development (i.e. especially during the cell division phase of fruit development, such as in tomato the first 10-14 days after fertilization).

Preferably the plants producing larger fruits as described above are homozygous for the mutant slarf9 allele, although heterozygous plants may also produce significantly larger fruits. To generate plants comprising the mutant allele in homozygous form, selfing can be used, optionally combined with genotyping (detecting the presence of the mutant allele e.g. by PCR using allele specific primers and/or sequencing). If TILLING populations are used the mutant plants (M1) are preferably selfed one or more times to generate for example M2 populations or preferably M3 or M4 populations for phenotyping. In M2 populations the mutant allele is present in a ratio of 1 (homozygous for mutant allele):2 (heterozygous for mutant allele):1 (homozygous for wild type allele). Segregation of fruit size correlates with segregation of the mutant allele.

The plant comprising the mutant slarf9 allele, or a variant thereof, and producing significantly larger fruits may be of any species, as the tomato sequences provided herein can be used to generate and identify plants comprising mutations in homologs and orthologs of the gene, as described further below. The endogenous SlARF9 variant nucleic acid sequence in the plant can be identified, which can then be used as target gene in the generation and/or identification of plants comprising a mutant allele of the SlARF9 variant. Thus, the mutant plant (i.e. the plant comprising a mutant slarf9 allele) may be a dicotyledonous or monocotyledonous species. Preferably the plant is a cultivated plant, although it is also an embodiment herein to identify mutant alleles in wild plants or non-cultivated plants and transfer these by breeding techniques into cultivated plants.

In one embodiment, the plant comprising at least one mutant slarf9 allele (in homozygous or heterozygous form) and having significantly larger fruits is of the family Solanaceae, i.e. encompassing the genera Solanum, Capsicum, Nicotiana and others or Cucurbitaceae (encompassing Cucumis species such as melon and cucumber). In another embodiment the plant is of the genus Solanum, e.g. encompassing cultivated tomato, potato, eggplant, and others.

In a specific embodiment the plant is of the species S. lycopersicum. Any S. lycopersicum may be generated and/or identified having at least one mutant slarf9 allele in its genome and producing larger fruits as a result. The tomato plant may, thus, be any cultivated tomato, any commercial variety, any breeding line or other, it may be determinate or indeterminate, open pollinated or hybrid, producing fruits of any color, shape and size. The mutant allele generated and/or identified in a particular tomato plant, or in a sexually compatible relative of tomato, may be easily transferred into any other tomato plant by breeding (crossing with a plant comprising the mutant allele and then selecting progeny comprising the mutant allele).

The plant may be any species of the family Solanaceae or of the genus Solanum, which species is either mutagenized itself to generate the mutant allele (e.g. by TILLING or other methods) or in which one or more natural or spontaneous mutations in the slarf9 gene (or variant) is/are identified, e.g. by Ecotilling.

It is also an embodiment of the invention to use Zinc-finger nucleases to generate mutations in endogenous SlARF9 genes as described in Townsend et al. 2009, Nature 459: 442-445 and by Shukla et al. 2009, Nature 459: 437-441.

The mutant allele is in one embodiment generated or identified in a cultivated plant, but may also be generated and/or identified in a wild plant or non-cultivated plant and then transferred into an cultivated plant using e.g. crossing and selection (optionally using interspecific crosses with e.g. embryo rescue to transfer the mutant allele). Thus, a mutant slarf9 allele may be generated (human induced mutation using mutagenesis techniques to mutagenize the target slarf9 gene or variant thereof) and/or identified (spontaneous or natural allelic variation) in other Solanum species include for example wild relatives of tomato, such as S. cheesmanii, S. chilense, S. habrochaites (L. hirsutum), S. chmielewskii, S. lycopersicum×S. peruvianum, S. glandulosum, S. hirsutum, S. minutum, S. parviflorum, S. pennellii, S. peruvianum, S. peruvianum var. humifusum and S. pimpinellifolium, and then transferred into a cultivated Solanum plant, e.g. Solanum lycopersicum by traditional breeding techniques. The term “traditional breeding techniques” encompasses herein crossing, selfing, selection, double haploid production, embryo rescue, protoplast fusion, transfer via bridge species, etc. as known to the breeder, i.e. methods other than genetic modification by which alleles can be transferred.

Preferably the mutation(s) in the slarf9) allele cause(s) the plant to have significantly larger fruits compared to plants lacking the mutant allele(s) (i.e. comprising wild type SlARF9 alleles), as described above.

Without limiting the invention, the mutation in SlARF9 (SEQ ID NO: 1, or variants thereof, or in the corresponding genomic sequence, e.g. the genomic sequence of SEQ ID NO: 3 nucleotides 2005-5879 or SEQ ID NO: 4 nucleotides 1196-5869, or variants thereof), result in reduced functionality or loss-of function of the SlARF9 protein, for example through single base transition(s), mis-sense or non-sense mutations, or insertion or deletion of one or more amino acids or a frame-shift in the coding sequence, which in turn results in the changed phenotype. The presence and type of mutation(s) can be analyzed by sequencing the gene, using SlARF9 and/or slarf9) specific primers. A “significant reduction” of the SlARF9 protein's functionality is preferably determined indirectly in vivo by the phenotype (i.e. significantly larger fruits) in plants heterozygous or, preferably, homozygous for the mutant allele. The enhanced fruit size phenotype co-segregates with the mutant allele.

In one embodiment of the invention a plant (preferably a tomato plant) is provided, which comprises one or more mutations in SEQ ID NO:1, SEQ ID NO: 3 (nucleotides 2005-5879) or SEQ ID NO: 4 (nucleotides 1196-5869), or in a nucleic acid sequence comprising at least about 60%, 62%, 65%, 70%, 80%, 90%, 95%, 97%, 97.5%, 98%, 98.5%, 99% or more sequence identity to any of these sequences (as defined), whereby the mutation results in the encoded SlARF9 protein (or variant) having reduced activity (compared to the wild type functional protein) or no activity in vivo and wherein said plant produces significantly larger fruits compared to a plant (preferably tomato) comprising a nucleic acid sequence encoding a wild type, functional SlARF9 protein (or variant).

In one embodiment a plant (preferably a tomato plant) is provided, which comprises one or more mutations in the nucleotide sequence encoding the protein of SEQ ID NO: 2, or a protein comprising at least 53%, 54%, 55%, 58%, 60%, 70%, 80%, 90%, 95%, 98%, 99% or more sequence identity to SEQ ID NO: 2 (as defined), and wherein the (tomato) plant comprises significantly larger fruits compared to a (tomato) plant lacking said one or more mutations.

In one embodiment a larger-fruit producing plant (preferably a tomato plant) comprising a mutant slarf9 allele is provided, characterized in that the mutation is a loss-of-function or reduced-function mutation of the encoded SlARF9 protein, said protein being a protein comprising at least 53%, 54%, 55%, 58%, 60%, 70%, 80%, 90%, 95%, 98%, 99% or more amino acid sequence identity to SEQ ID NO: 2.

The plant (e.g. the tomato plant) is preferably homozygous for the mutant SLARF9 allele.

In one aspect, a Solanum lycopersicum plant comprising a mutant slarf9) allele in its genome, especially the allele having one or more single point mutations, in any one of the exons of SEQ ID NO: 3 (or a variant thereof) is provided, especially in exon 2 and/or exon 7. In one aspect the mutation is in the codon sequence of one of the following amino acids: amino acid 52, 191 and/or 193 of SEQ ID NO: 2. Tomato plants comprising an mutant slarf9 allele and producing significantly larger fruits, wherein the mutant allele comprises one or more of the following mutations, are an embodiment of the invention (denoting first the amino acid in the wild type SlARF9 protein being converted into a different amino acid in the mutant at the position indicated by subscript): Gly₅₂→Ser₅₂, Arg₁₉₁→Trp₁₉₁ or His₁₉₃→Tyr₁₉₃. In one aspect the mutation is in the sequence encoding a conserved domain of the SlARF9 protein, especially in the b3-derived DNA binding domain, i.e. amino acids 74 to 236 of SEQ ID No. 2 or of a variant thereof. In one aspect, tomato plants comprising a mutant slarf9) allele, as obtainable from seeds deposited under NCIMB 41827, 41828, 41829, 41839 and/or 41831 (or from plants derived from such seeds or from progeny of these plants) are provided herein, whereby the fruits of the tomato plants are significantly larger than the fruits of tomato plants having wild type SlARF9 alleles at the SlARF9 locus.

In another embodiment the plant comprising the endogenous mutant slarf9 allele is a watermelon plant, producing significantly larger watermelon fruits as a result, compared to watermelon plants comprising the wild type SlARF9 allele. In another embodiment the plant is a cucumber plant, melon plant or pepper plant.

Mutant plants can be distinguished from non-mutants by molecular methods, such as the mutation(s) present in the slarf9 genomic DNA or mRNA (cDNA), SlARF9 protein levels and/or protein activity, etc., and by the modified phenotypic characteristics compared to the wild type. The mutant allele can be transferred into other plants which are sexually compatible with the mutant plant using traditional crossing and selection. Thus, the mutant allele can be used to generate large-fruited tomato varieties of any type, e.g. open pollinate varieties, hybrid varieties, F1 hybrids, Roma type, cherry type, determinate or indeterminate types, etc. In one embodiment the plant (preferably S. lycopersicum) comprising the mutant slarf9) allele and having significantly larger fruits is an F1 hybrid plant or a F1 seed, from which an F1 hybrid plant is grown. The inbred parents used to make the F hybrid preferably both comprise the same mutant slarf9 allele in their genome in homozygous form.

In another embodiment, the plant comprising the mutant slarf9) allele (e.g. tomato) is crossed with another plant of the same species or of a closely related species, to generate a hybrid plant (hybrid seed) comprising the mutant slarf9 allele. Such a hybrid plant is also an embodiment of the invention. Also a method for transferring a mutant slarf9) allele to another plant is provided, comprising providing a plant comprising a mutant slarf9 allele in its genome, whereby the mutant allele confers larger fruit size (as described above), crossing said plant with another plant and obtaining the seeds of said cross. Optionally plants obtained from these seeds may be further selfed and/or crossed and progeny selected comprising the mutant allele and producing significantly larger fruits due to the presence of the mutant allele compared to plants comprising the wild type SlARF9 allele.

In one embodiment, the parents used to make the F1 hybrid each comprise different mutant slarf9 alleles in homozygous form, so that the hybrid comprises two different mutant slarf9 alleles. For example, parent 1 may comprise loss-of-function mutant while parent 2 comprises a reduced-function mutant. The F1 hybrid then comprises one allele from each parent. Thus, also tomato plants comprising two different mutant slarf9 alleles at the SlARF9 locus and producing significantly larger fruits are encompassed herein.

In one aspect, the mutant allele as obtainable from seeds deposited under NCIMB 41827, 41828, 41829, 41839 and/or 41831 (or from plants derived from such seeds or from progeny of these plants) may be homozygous in the plant, or may be combined with a wild type allele or with another mutant slarf9 allele, such as e.g. any of the alleles obtainable from seeds deposited under NCIMB 41827, 41828, 41829, 41839 and/or 41831 (or from plants derived from such seeds or from progeny of these plants). Thus, a tomato plant comprising the allele encoding the mutation Gly₅₂→Ser₅₂, may be combined with an allele Arg₁₉₁→Trp₁₉₁ or His₁₉₃→Tyr₁₉₃. Similarly, mutant alleles encoding Arg₁₉₁→Trp₁₉₁ and His₁₉₃→Tyr₁₉₃ may be combined in one plant. Also a splice-site mutant allele, such as obtainable from seeds deposited under Accession number NCIMB 41827 may be homozygous or in heterozygous form and optionally combined with a wild type SlARF9 allele or with another mutant slarf9 allele, such as an allele encoding the mutation Gly₅₂→Ser₅₂, Arg₁₉₁→Trp₁₉₁ or His₁₉₃→Tyr₁₉₃, as obtainable from seeds deposited under NCIMB 41828, 41829, 41839 and/or 41831 (or from plants derived from such seeds or from progeny of these plants).

Plants comprising a mutant slarf9 allele, encoding a loss-of-function or reduced-function protein (e.g. a truncated protein as a result of a non-sense mutation, a protein having a modified amino acid sequence, resulting e.g. in a modified catalytic site, a modified folding, etc., for example due to a mis-sense mutation, frame-shift mutation and/or a splice site mutation), can be generated and/or identified by using mutagenesis methods or by screening natural populations for natural variants in the slarf9 allele. In one embodiment of the invention TILLING is used to generate such plants and/or to identify such mutagenesis induced mutations and/or EcoTILLING is used to identify plants, such as wild plants or non-cultivated plants, comprising natural (spontaneous) mutations in the slarf9 gene, which can then be transferred into cultivated plants by traditional breeding techniques. However, any other mutagenesis method may be used and it is understood that both human induced mutants, UV or X-ray mutagenesis, chemical mutagens etc. and spontaneous mutants of the slarf9 gene generated in or transferred into cultivated plants or crop plants by traditional breeding are encompassed herein. Also targeted mutagenesis, using for example zinc finger endonucleases, can be used to mutate the endogenous SlARF9 gene and to generate slarf9 alleles encoding reduced-function or loss-of function SlARF9 proteins or mutations in the endogenous SlARF9 promoter leading to reduced or no SlARF9 protein being made at least during fruit development.

In one specific embodiment according to the invention the mutant plant (i.e. the plant comprising the mutant slarf9 allele) is a plant of a different species than tomato, e.g. a monocotyledonous cultivated plant, preferably a rice, maize, wheat or barley plant comprising a mutant slarf9 allele in its genome, or a vegetable- or fruit species, such as watermelon, melon, cucumber, pepper, squash, pumpkin, strawberry, apple, peach, cherry, plum, grape, lemon, orange, pear, raspberry, gooseberry, blueberry, etc. When using methods such as TILLING, the amplification of the target gene fragment may be based on SEQ ID NO: 1, or fragments thereof (e.g. using specific or degenerate primers, for example designed based on one or more of the conserved domains of SlARF9), or one may first isolate the SlARF9 ortholog and base primer design on the orthologous sequence. Primers for amplifying the target gene fragment may also be based on intron sequences or intron-exon boundary sequences. For example when a mutation in a large exon is screened for, the exon may be amplified using two PCR reactions and two primer pairs, whereby one or more of the primers may lie in the intron sequences flanking the exon. Primer pairs may, therefore, also be based on the genomic sequence of SlARF9, such as depicted in SEQ ID NO: 3 (especially nucleotides from about 1977 to 5940, or 2005 to 5879) and SEQ ID NO: 4 (especially nucleotides from about 1950 to 5909, or from 1996 to 5869).

TILLING (Targeting Induced Local Lesions IN Genomes) is a general reverse genetic technique that uses traditional chemical mutagenesis methods to create libraries of mutagenized individuals that are later subjected to high throughput screens for the discovery of mutations. TILLING combines chemical mutagenesis with mutation screens of pooled PCR products, resulting in the isolation of mis-sense and non-sense mutant alleles of the targeted genes. Thus, TILLING uses traditional chemical mutagenesis (e.g. EMS or MNU mutagenesis) or other mutagenesis methods (e.g. radiation such as UV) followed by high-throughput screening for mutations in specific target genes, such as SlARF9 according to the invention. 51 nucleases, such as CEL1 or ENDO1, are used to cleave heteroduplexes of mutant and wildtype target DNA and detection of cleavage products using e.g. electrophoresis such as a LI-COR gel analyzer system, see e.g. Henikoff et al. Plant Physiology 2004, 135: 630-636. TILLING has been applied in many plant species, such as tomato (see http://tilling.ucdavis.edu/index.php/Tomato_Tilling), rice (Till et al. 2007, BMC Plant Biol 7: 19), Arabidopsis (Till et al. 2006, Methods Mol Biol 323: 127-35), Brassica, maize (Till et al. 2004, BMC Plant Biol 4: 12), etc. Also EcoTILLING, whereby mutants in natural populations are detected, has been widely used, see Till et al. 2006 (Nat Protoc 1: 2465-77) and Comai et al. 2004 (Plant J 37: 778-86). In one embodiment herein, classical TILLING is modified and instead of using enzyme based mutant detection (enzymatic digestion with a single-strand specific nuclease and high resolution polyacrylamide gel electrophoresis), two different high throughput detection systems can be used which have previously only been used in humans. These detection protocols are adaptations of CSCE (Conformation Sensitive Capillary Electrophoresis, see Rozycka et al. 2000, Genomics 70, 34-40) or of HRM (High Resolution Melting, see Clin Chem 49, 853-860). See Gady et al. 2009, Plant Methods 5:13.

Thus, non-transgenic plants, seeds, fruits and tissues comprising a mutant slarf9 allele in one or more tissues and comprising one or more of the phenotypes conferred by a reduced-function or loss-of-function SlARF9 protein according to the invention (e.g. larger fruits as described above) and methods for generating and/or identifying such plants is encompassed herein.

Also a method for generating and/or identifying a mutant slarf9 allele suitable for generating plants that produce larger fruits and/or a method for generating a plant that produces larger fruits is provided, comprising the steps of:
(a) mutagenizing plant seeds (e.g. by EMS mutagenesis) to generate an M1 population or providing mutagenized plant seeds or providing plants comprising natural variation,
(b) optionally selfing the plants of (a) one or more times to generate an M2, M3 or M4 families,
(c) preparing DNA of the plants of (a) or (b) and pooling DNA of individuals, or pooling tissue samples of individuals and preparing DNA from the pooled samples,
(d) PCR amplification of all or part of the SlARF9 target gene (genomic or cDNA), or a variant thereof, from the DNA pools or from DNA from the pooled tissue samples,
(e) detecting the presence of mutated slarf9 allele(s) in the PCR amplification products and thereby also in the DNA pools or in the DNA from the pooled tissue samples,
(f) selecting the corresponding individual plants comprising the mutant slarf9 allele(s),
(g) optionally sequencing the mutant slarf9 allele of the plant;
(h) phenotyping the plants of (f), or progeny thereof, for larger fruit size production, and
(i) selecting plants producing larger fruits than the controls, and optionally
(j) breeding with the plant of (i) to generate cultivated plants producing large fruits and having good agronomic characteristics.

Between steps (e) and (f) optionally SIFT analysis may be carried out in order to predict which mutation will result in conferring larger fruit size on the plants.

In step (d) primers which amplify all or part of the target gene, SlARF9 (SEQ ID NO: 1, SEQ ID NO: 3 or 4) or a variant thereof, are designed using standard methods, such as CODDLE (http://www.proweb.org/doddle). Primers may be designed to amplify e.g. at least about 50, 100, 200, 250, 300, 400, 500, 600, 800 bp or at least about 1000 bp or more of the target gene, i.e. of SEQ ID NO: 1, 3 or 4, or of a variant of SEQ ID NO: 1, 3 or 4. Preferably a fragment comprising all or part of a conserved domain of the SlARF9 protein is amplified by the primer, e.g. the fragment encodes all or part of the DNA-binding domain, of the MR, of the dimerization domain III or of the dimerization domain IV.

For plant species other than tomato, it may be desirable to first identify the sequence of the endogenous SlARF9 gene in order to be able to design good primer sequences. The sequence may be identified in silico or by, for example, designing degenerate PCR primers and amplifying all or part of the SlARF9gene variant (ortholog of the tomato SlARF9 gene) from the genome of the plant species. The sequence of the endogenous SlARF9 gene is then preferably used to design suitable primers for TILLING.

Step (e) may make use of S1 nucleases, such as CEL1, to detect mismatches between the PCR amplification product, i.e. between the wild type SlARF9 PCR product and the mutant slarf9 PCR product which form heteroduplexes. Alternatively, step (e) may use CSCE or HRM for detection. In CSCE homoduplexes (WT/WT or mutant/mutant fragments) are formed and heteroduplexes (mutant/WT fragments). Because of the mismatch formed, heteroduplexes migrate at a different speed than the homoduplexes through capillaries, thus allowing the identification of pools containing a mutation within the target fragment. HRM is also a non-enzymatic technique. During the PCR amplification of the target gene fragments LCgreen Plus+™ molecules are incorporated between each annealed base pair of the double stranded DNA molecule, which—when captured in the molecule—will emit fluorescence. A LightScanner records the fluorescence intensity while the plate is progressively heated. At a certain temperature the PCR products start to melt and release the LCgreen Plus+™, whereby fluorescence decreases. DNA pools containing a mutation (heteroduplexes) are identified because their melting temperature is lower than that of homoduplexes.

Step (j) may involve traditional breeding methods and phenotypic and/or marker assisted selection methods. Many different varieties which comprise one or more mutant slarf9 alleles and produce significantly larger fruits than plants comprising one or more wild type SlARF9 alleles can be generated this way.

Extensive protocols for carrying out TILLING have been published, see e.g. http://blocks.fhcrc.org%7Esteveh/TILLING_publications.html and Till et al. (2006) Nature Protocols 1:2465-2477; Till et al. (2006) Methods Mol Biol. 323:127-135 and Till et al. (2003) Methods Mol Biol. 236:205-220, all incorporated herein by reference.

Once a plant comprising a mutant allele which confers the desired phenotype has been identified, this allele can be transferred to other plants by traditional breeding techniques, e.g. by crossing the plant with another plant and collecting progeny of the cross. In step (j) the allele may thus be used to generate plants which produce large fruits and which provide good agronomic characteristics.

As mentioned, it is understood that other mutagenesis and/or selection methods may equally be used to generate mutant plants according to the invention. Seeds may for example be radiated or chemically treated to generate mutant populations. Also direct gene sequencing of slarf9 may be used to screen mutagenized plant populations for mutant alleles. For example KeyPoint screening is a sequence based method which can be used to identify plants comprising mutant slarf9 alleles (Rigola et al. PloS One, March 2009, Vol 4(3):e4761).

Thus, non-transgenic mutant plants which produce lower levels of (functional) wild type SlARF9 protein in one or more tissues (particularly at least in fruit tissue) are provided, or which completely lack functional SlARF9 protein in specific tissues or which produce a non-functional SlARF9 protein in certain tissues, e.g. due to mutations in one or more endogenous slarf9 alleles. These mutants may be generated by mutagenesis methods, such as TILLING or variants thereof, or they may be identified by EcoTILLING or by any other method. Slarf9 alleles encoding non-functional or reduced-functional SlARF9 protein may be isolated and sequenced or may be transferred to other plants by traditional breeding methods.

Any part of the plant, or of the progeny thereof, is provided, including harvested fruit, harvested tissues or organs, seeds, pollen, flowers, ovaries, etc. comprising a mutant slarf9 allele according to the invention in the genome. Also plant cell cultures or plant tissue cultures comprising in their genome a mutant slarf9 allele are provided. Preferably, the plant cell cultures or plant tissue cultures can be regenerated into whole plants comprising a mutant slarf9 allele in its genome. Also double haploid plants (and seeds from which double haploid plants can be grown), generated by chromosome doubling of haploid cells comprising an slarf9 mutant allele, and hybrid plants (and seeds from which hybrid plants can be grown) comprising a mutant slarf9 allele in their genome are encompassed herein, whereby the double haploid plants and hybrid plants produce significantly larger fruits according to the invention.

Also provided are kits for detecting whether or not a plant comprises a mutant slarf9 allele according to the invention. Such a kit may comprise PCR primers or probes detection of the allele in a tissue sample or in DNA or RNA obtained from such tissue.

Preferably, the mutant plants also have good other agronomic characteristics, i.e. they do not have reduced fruit numbers and/or reduced fruit quality compared to wild type plants. Preferably yield of such plants is higher due to fruits being larger. Also the larger number of cells and/or smaller cell size in the pericarp tissue results in solid content being higher (more cell walls per gram fresh weight). The soluble and insoluble solid content of the fruits is thus higher. In a preferred embodiment the plant is a tomato plant and the fruit is a tomato fruit, such as a processing tomato, fresh market tomato of any shape or size or colour. Thus, also harvested products of plants or plant parts comprising one or two mutant slarf9 alleles are provided. This includes downstream processed products, such as tomato paste, ketchup, tomato juice, cut tomato fruit, canned fruit, dried fruit, peeled fruit, etc. The same applies for other plant species. The products can be identified by comprising the mutant allele in their genomic DNA.

### Further Uses According to the Invention

According to the instant invention the use of a nucleic acid sequence encoding an SlARF9 protein for modifying fruit size of plants is provided. In a preferred embodiment, this use involves modifying (increasing or decreasing) the level of functional SlARF9 protein in the plant or in specific plant parts (e.g. at least in the fruits).

In one embodiment the use of a nucleic acid sequence encoding an SlARF9 protein for the generation of transgenic or non-transgenic plants which produce large fruits is provided herein, whereby the SlARF9 protein comprises at least 60%, 70%, 75%, 80%, 85%, 90%, 95%, 98% or 99% amino acid sequence identity to SEQ ID NO: 2. Herein, such use encompasses any use involving a plants, seeds or plant cells or tissues comprising in the genome a slarf9 allele according to the invention with the purpose of producing or using larger fruits.

Similarly, the use of a nucleic acid sequence encoding an SlARF9 protein for increasing fruit size in plants is provided, whereby the SlARF9 protein comprises at least 60%, 70%, 75%, 80%, 85%, 90%, 95%, 98% or 99% amino acid sequence identity to SEQ ID NO: 2.

In one aspect, the use of a plant or a seed comprising a mutant slarf9 allele for producing fruits with increased size is provided herein, whereby an slarf9 allele is an allele which encodes a protein comprising at least 60%, 70%, 75%, 80%, 85%, 90%, 95%, 98% or 99% amino acid sequence identity to SEQ ID NO: 2. As a result of said one or more mutations, the plant comprising said mutant allele in its genome produces significantly larger fruits compared to the plant comprising a wild type SlARF9 allele in its genome.

In another aspect, the use of an in vitro plant cell or tissue culture comprising a mutant slarf9 allele for producing plants which produce fruits with increased size is provided herein, whereby an slarf9 allele is an allele which encodes a protein comprising at least 60%, 70%, 75%, 80%, 85%, 90%, 95%, 98% or 99% amino acid sequence identity to SEQ ID NO: 2. The plant cells or tissue culture can be regenerated into a whole plant using known methods.

Likewise, the use of fruits of increased size, comprising in their genome a mutant slarf9 allele for harvest, storage, processing or sale is provided, whereby an slarf9 allele is an allele which encodes a protein comprising at least 60%, 70%, 75%, 80%, 85%, 90%, 95%, 98% or 99% amino acid sequence identity to SEQ ID NO: 2

Also the use of a nucleic acid sequence encoding an SlARF9 protein for the generation of transgenic or non-transgenic plants which produce smaller fruits is provided herein. The SlARF9 protein expression is increased as described elsewhere herein and the SlARF9 protein is a functional protein and comprises at least 60%, 70%, 75%, 80%, 85%, 90%, 95%, 98% or 99% amino acid sequence identity to SEQ ID NO: 2.

The plant is preferably of the genus Solanum, Capsicum or Cucumis. In one aspect, the plant is preferably tomato, pepper, cucumber or melon.

In one aspect the mutant slarf9 allele is an allele obtainable from plants grown from seeds deposited under Accession number NCIMB 41827, 41828, 41829, 41830 or 41831.

Thus, in one aspect, the use of a nucleic acid sequence encoding a mutant slarf9 allele for the generation of non-transgenic plants which produce large fruits is provided herein and in one aspect the mutant slarf9 allele is an allele as obtainable from the above seed deposits.

### Sequences

SEQ ID NO 1: cDNA sequence of the wild type SlARF9 allele from tomato cv Moneymaker
SEQ ID NO 2: protein sequence of the wild type SlARF9 protein encoded by SEQ ID NO: 1. Amino acids 74-236 comprise the B3-derived DNA binding domain. Amino acids 237-564 comprise the middle region (MR). Amino acids 256-332 comprise a putative auxin response region. Amino acids 565-602 comprise the dimerization domain III. Amino acids 609-651 comprise the dimerization domain IV.
SEQ ID NO 3: Promoter region (nucleotides 1-2004) and genomic DNA of wild type SlARF9 from tomato cv Moneymaker. Transcription (mRNA) start is at nucleotide 1551 and transcription stop at 6323, with the translation start codon being the ATG at position 2005-2007 and the translation stop codon being the TAA at position 5877-5879. The 5′UTR is thus from base 1551 to 2004 and the 3′UTR from base 5880 to 6323.
SEQ ID NO 4: Promoter region (nucleotides 1-1995) and genomic DNA of wild type SlARF9 from tomato cv Heinz1706. Transcription (mRNA) start is at nucleotide 1543 and transcription stop at 6313, with the translation start codon being the ATG at position 1996-1998 and the translation stop codon being the TAA at position 5867-5869. The 5′UTR is thus from base 1543 to 1995 and the 3′UTR from base 5870 to 6313.
SEQ ID NO 5: actin primer, forward
SEQ ID NO 6: actin primer, reverse
SEQ ID NO 7: SlARF9 primer, forward, for mRNA detection
SEQ ID NO 8: SlARF9 primer, reverse, for mRNA detection
SEQ ID NO 9: SlARF9 primer, forward, for coding sequence amplification
SEQ ID NO 10: SlARF9 primer, reverse, for coding sequence amplification
SEQ ID NO 11: SlARF9 primer, forward, for RNAi fragment amplification
SEQ ID NO 12: SlARF9 primer, revers, for RNAi fragment amplification
SEQ ID NO 13: Promoter SlARF9 primer, forward, for promoter amplification
SEQ ID NO 14: Promoter SlARF9 primer, reverse, for promoter amplification
SEQ ID NO 15: Forward primer for screening plant populations for mutations in Exon 2
SEQ ID NO 16: Reverse primer for screening plant populations for mutations in Exon 2
SEQ ID NO 17: Forward primer for screening plant populations for mutations in Exon 2
SEQ ID NO 18: Reverse primer for screening plant populations for mutations in Exon 2
SEQ ID NO 19: Forward primer for screening plant populations for mutations in Exon 6
SEQ ID NO 20: Reverse primer for screening plant populations for mutations in Exon 6
SEQ ID NO 21: Forward primer for screening plant populations for mutations in Exon 7
SEQ ID NO 22: Reverse primer for screening plant populations for mutations in Exon 7

### FIGURE LEGENDS

FIG. 1—relative SlARF9 mRNA levels during tomato fruit set

(a) SlARF9 expression pattern by real-time quantitative PCR, 1, 2 and 3 days (d) after treatment, in placenta together with ovular tissue and the ovary wall. Total RNA was isolated from emasculated flowers (Control), from emasculated flowers treated with gibberellic acid (GA3), and from emasculated flowers after hand pollination (Pollinat.).

(b) Relative mRNA levels of SlARF9 in tomato ovaries collected throughout seven different stages of flower development. At stages 1-4, flower bud sizes were as indicated. Stage 4 represents the flower at the stage of emasculation. At stage 5, the flower is fully open (Anthesis). For stage 6, flowers were collected at 3 d after anthesis (DAA). For stage 7, flowers were collected 3 d after hand pollination. Standard errors are indicated (n=2).

(c) Relative mRNA levels of SlARF9 in unpollinated tomato ovaries at anthesis and ovaries collected 3 d after hand pollination, dissected into ovules, placenta and ovary wall tissue samples. Standard errors are indicated (n=2).

(d) Relative mRNA levels of SlARF9 in tomato ovaries of emasculated flowers collected 6 or 24 h after auxin treatment (IAA). Untreated ovaries were used as a control. Standard errors are indicated (n=2).

(e) Relative mRNA levels of SlARF9 in young flower buds, unpollinated ovaries and various other floral organs collected from flowers at the stage of emasculation, pollinated ovaries (3 DAP), and in the hypocotyl and root of 10 d old seedlings. Standard errors are indicated (n=2). In each of the above figures (a) to (e), the highest value was set to equal 1.

FIG. 2: SlARF9 mRNA levels in developing wild-type and transgenic fruits

(a) Relative mRNA levels of SlARF9 in ovaries and fruits collected from wild-type and SlARF9-OE lines. Standard errors are indicated (n=2). Wild type levels in 3-4 mm fruit (6 DAP) were set to 1.

(b) Relative mRNA levels of SlARF9 in ovaries and fruits collected from wild-type and RNAi SlARF9 lines. Standard errors are indicated (n=2). Wild type levels in 3-4 mm fruit (6 DAP) were set to 1.

FIG. 3:

Photograph of a tomato fruit from a SlARF9-RNAi plant (left) and from the wild type control (right).

FIG. 4:

Micrograph of pericarp cells of a tomato fruit (10 DAP) from a SlARF9-RNAi plant (left) and from the wild type control (right).

The following non-limiting Examples describe the use of SlARF9 and slarf9 genes for modifying plant phenotypes. Unless stated otherwise in the Examples, all recombinant DNA techniques are carried out according to standard protocols as described in Sambrook et al. (1989)Molecular Cloning: A Laboratory Manual, Second Edition, Cold Spring Harbor Laboratory Press, and Sambrook and Russell (2001) Molecular Cloning: A Laboratory Manual, Third Edition, Cold Spring Harbor Laboratory Press, NY; and in Volumes 1 and 2 of Ausubel et al. (1994) Current Protocols in Molecular Biology, Current Protocols, USA. Standard materials and methods for plant molecular work are described in Plant Molecular Biology Labfax (1993) by R. D. D. Croy, jointly published by BIOS Scientific Publications Ltd (UK) and Blackwell Scientific Publications, UK.

### Seed Deposits

A representative sample of seeds of five tomato TILLING mutants according to Example 3, were deposited by Nunhems B. V. on 14 Apr. 2011 at the NCIMB Ltd. (Ferguson Building, Craibstone Estate, Bucksburn Aberdeen, Scotland AB21 9YA, UK) according to the Budapest Treaty, under the Expert Solution (EPC 2000, Rule 32(1)). Seeds were given the following deposit numbers: NCIMB 41827 (mutant 1719), NCIMB 41828 (mutant 2484), NCIMB 41829 (mutant 3175), NCIMB 41830 (mutant 6725) and NCIMB 41831 (mutant 6932).

The Applicant requests that samples of the biological material and any material derived therefrom be only released to a designated Expert in accordance with Rule 32(1) EPC or related legislation of countries or treaties having similar rules and regulation, until the mention of the grant of the patent, or for 20 years from the date of filing if the application is refused, withdrawn or deemed to be withdrawn.

SEQ ID NO: 1 Solanum lycopersicum ARF9 coding
sequence from cv Moneymaker
atggcaacta taaatgggtg gtgttatgag tctcagccga atatgaattc tccaggtaaa
aaagatgctc tgtatcatga gctatggcag ttgtgtgcag gtccagtagt tgatgttccc
agggaaggag aaagagttta ttattttcct caaggccaca tggaacaatt ggtagcatca
attaatcaag aaatggatca aagagttcca tcattcaatc tcaaatcaaa ggtcctttgt
cgagttatca atagtcattt cctggctgaa gaagacaatg atgaggtcta tgtccagatc
actttgatgc cagaggcacc acatgtaccc gagccgacta ctccggatcc attaattccg
caggatgtaa agcctagatt ccattctttc tgcaaggtcc tgacagcctc cgatacaagc
actcatggtg gattttctgt tctaaggaaa catgctaatg aatgccttcc tccattggac
ttgaaccagc agactccgac ccaggaattg attgcgaaag accttcatga cgtggagtgg
cgcttcaagc atatatttag aggccaacct cggagacatt tacttaccac agggtggagt
acctttgttt cttcaaagaa attagtggca ggggattctt ttgtattctt gaggggtaat
aatggacagc tgcgagttgg ggtcaaaagg cttgttcgcc agcagagctc aatgccgtca
tcggtgatgt caagccagag catgcaccta ggagtcttgg ctacagcatc tcatgctgtt
acaacccaga caatgtttgt tgtttactac aaaccgagaa ccactcagtt catcgtaggc
gtcaacaaat acttagaggc tcttaaacat gaatatgcag ttggcatgcg attcaaaatg
cagtttgaag ccgaagggaa tcctgataga agatttatgg gcactatagt tggaattgat
gatctttctt cacagtggaa aaattctgcg tggcgatcct tgaaggtccg atgggacgag
cctgcagcca ttgcaaggcc tgacagagtt tctccttggg aaattaaacc ttatgtgtgt
tcaattccaa atgtccttgt cccaccaacc gcagagaaga acaaaaggca tcggctacat
agtgaaatca aaatatcaga acaaccttca tcctcaaatg cttcggcggt ttggaatcct
tcccttcgat cgcctcagtt taacaccttt ggcatcaaca gcagtactaa ttgcgcatta
gcgtctctta cagagagtgg ttggcagctt cctcatttaa atacttcagg tatgcttgtg
gatgagccag aagacggtag gagtgctcca acttggtgtg gttttccatg cgttttggcc
ccgcagttcg gtcaagggac taatcagccg attgttattc ctactgatgg gagaaaatgt
gataccaaaa aaacctgtag attatttggt attgacttga aaagttcctc aattagcact
actgaggcac gactacagct acagccagcc ggcatttctt gtgtctttgc agagagagca
cctccaaaca cggtgcctgc tggtgattca gatcaaaagt ctgagctttc agtagacttc
aaagatcaaa tgcaaggcca tttgcggtta cccctaaagg aggttcaaag caagcagagt
tgttccacca ggtctcgcac aaaggtgcaa atgcaaggcg tagctgtagg tcgtgcagtg
gatttaacca tattgaaagg atacgatgag cttacaaagg agcttgagga gatgtttgaa
atccaaggag agcttcagtc acgacagaaa tgggggatct tgtttacaga tgatgaaggg
gatacaatgc ttatgggtga ttatccgtgg caagactttt gcaatgtggt gaggaagatt
ttcatttgtt caagtcagga tatgaaaaaa ttgaccctgt ctcgcgcaga ccattaa

SEQ ID NO: 2—Solanum lycopersicum ARF9 protein from cv Moneymaker
DOMAIN (74) . . . (236) B3 derived DNA binding domain

### DOMAIN (237) . . . (564) Middle Region (MR)

DOMAIN (256) . . . (332) auxin response region, part of the Middle Region (MR)
DOMAIN (565) . . . (602) Dimerization domain III
DOMAIN (609) . . . (651) Dimerization domain IV

```
Met Ala Thr Ile Asn Gly Trp Cys Tyr Glu Ser Gln Pro Asn Met Asn
1 5 10 15
Ser Pro Gly Lys Lys Asp Ala Leu Tyr His Glu Leu Trp Gln Leu Cys
                        20 25 30
Ala Gly Pro Val Val Asp Val Pro Arg Glu Gly Glu Arg Val Tyr Tyr
                35 40 45
Phe Pro Gln Gly His Met Glu Gln Leu Val Ala Ser Ile Asn Gln Glu
        50 55 60
Met Asp Gln Arg Val Pro Ser Phe Asn Leu Lys Ser Lys Val Leu Cys
65 70 75 80
Arg Val Ile Asn Ser His Phe Leu Ala Glu Glu Asp Asn Asp Glu Val
                                85 90 95
Tyr Val Gln Ile Thr Leu Met Pro Glu Ala Pro His Val Pro Glu Pro
                        100 105 110
Thr Thr Pro Asp Pro Leu Ile Pro Gln Asp Val Lys Pro Arg Phe His
                115 120 125
Ser Phe Cys Lys Val Leu Thr Ala Ser Asp Thr Ser Thr His Gly Gly
        130 135 140
Phe Ser Val Leu Arg Lys His Ala Asn Glu Cys Leu Pro Pro Leu Asp
145 150 155 160
Leu Asn Gln Gln Thr Pro Thr Gln Glu Leu Ile Ala Lys Asp Leu His
                                165 170 175
Asp Val Glu Trp Arg Phe Lys His Ile Phe Arg Gly Gln Pro Arg Arg
                        180 185 190
His Leu Leu Thr Thr Gly Trp Ser Thr Phe Val Ser Ser Lys Lys Leu
                195 200 205
Val Ala Gly Asp Ser Phe Val Phe Leu Arg Gly Asn Asn Gly Gln Leu
        210 215 220
Arg Val Gly Val Lys Arg Leu Val Arg Gln Gln Ser Ser Met Pro Ser
225 230 235 240
Ser Val Met Ser Ser Gln Ser Met His Leu Gly Val Leu Ala Thr Ala
                                245 250 255
Ser His Ala Val Thr Thr Gln Thr Met Phe Val Val Tyr Tyr Lys Pro
                        260 265 270
Arg Thr Thr Gln Phe Ile Val Gly Val Asn Lys Tyr Leu Glu Ala Leu
                275 280 285
Lys His Glu Tyr Ala Val Gly Met Arg Phe Lys Met Gln Phe Glu Ala
        290 295 300
Glu Gly Asn Pro Asp Arg Arg Phe Met Gly Thr Ile Val Gly Ile Asp
305 310 315 320
Asp Leu Ser Ser Gln Trp Lys Asn Ser Ala Trp Arg Ser Leu Lys Val
                                325 330 335
Arg Trp Asp Glu Pro Ala Ala Ile Ala Arg Pro Asp Arg Val Ser Pro
                        340 345 350
Trp Glu Ile Lys Pro Tyr Val Cys Ser Ile Pro Asn Val Leu Val Pro
                355 360 365
Pro Thr Ala Glu Lys Asn Lys Arg His Arg Leu His Ser Glu Ile Lys
        370 375 380
Ile Ser Glu Gln Pro Ser Ser Ser Asn Ala Ser Ala Val Trp Asn Pro
385 390 395 400
Ser Leu Arg Ser Pro Gln Phe Asn Thr Phe Gly Ile Asn Ser Ser Thr
                                405 410 415
Asn Cys Ala Leu Ala Ser Leu Thr Glu Ser Gly Trp Gln Leu Pro His
                        420 425 430
Leu Asn Thr Ser Gly Met Leu Val Asp Glu Pro Glu Asp Gly Arg Ser
                435 440 445
Ala Pro Thr Trp Cys Gly Phe Pro Cys Val Leu Ala Pro Gln Phe Gly
        450 455 460
Gln Gly Thr Asn Gln Pro Ile Val Ile Pro Thr Asp Gly Arg Lys Cys
465 470 475 480
Asp Thr Lys Lys Thr Cys Arg Leu Phe Gly Ile Asp Leu Lys Ser Ser
                                485 490 495
Ser Ile Ser Thr Thr Glu Ala Arg Leu Gln Leu Gln Pro Ala Gly Ile
                        500 505 510
Ser Cys Val Phe Ala Glu Arg Ala Pro Pro Asn Thr Val Pro Ala Gly
                515 520 525
Asp Ser Asp Gln Lys Ser Glu Leu Ser Val Asp Phe Lys Asp Gln Met
        530 535 540
Gln Gly His Leu Arg Leu Pro Leu Lys Glu Val Gln Ser Lys Gln Ser
545 550 555 560
Cys Ser Thr Arg Ser Arg Thr Lys Val Gln Met Gln Gly Val Ala Val
                                565 570 575
Gly Arg Ala Val Asp Leu Thr Ile Leu Lys Gly Tyr Asp Glu Leu Thr
                        580 585 590
Lys Glu Leu Glu Glu Met Phe Glu Ile Gln Gly Glu Leu Gln Ser Arg
                595 600 605
Gln Lys Trp Gly Ile Leu Phe Thr Asp Asp Glu Gly Asp Thr Met Leu
        610 615 620
Met Gly Asp Tyr Pro Trp Gln Asp Phe Cys Asn Val Val Arg Lys Ile
625 630 635 640
Phe Ile Cys Ser Ser Gln Asp Met Lys Lys Leu Thr Leu Ser Arg Ala
                                645 650 655
Asp His
```

### EXAMPLES

### Example 1

### Isolation and Characterization of SlARF9

### 1.1 Materials and Methods

### 1.1.1 Plant Materials and Growth Conditions

The tomato plants (Solanum lycopersicum cv. Moneymaker) were grown as described in de Jong et al. (2009, The Plant Journal 57, 160-170). Also the in vitro culture was performed following the protocol in de Jong et al. (2009, supra). For expression analysis of SlARF9 in ovaries, flowers were emasculated 3 days (d) before anthesis. Hand pollination or hormone treatments were carried out at the stage of anthesis. SlARF9 expression under the influence of auxin was analysed in ovaries of flowers treated with 2 μl of 1 mM 4-C1-IAA (Sigma-Aldrich, http://www.sigmaaldrich.com) in 2% ethanol. The treatment was repeated 6 h after the first application. Control flowers were collected at the stage of anthesis.

For analysis of SlARF9 expression in the transgenic lines, pericarp tissue was collected from ovaries and fruit that were formed by the second generation (T2) of the SlARF9-OE lines (overexpressing lines), and the first generation (T1) of RNAi SlARF9 lines. All collected tissues were frozen in N2 and stored at −80° C. until RNA extraction.

### 1.1.2 Real-Time Quantitative PCR

Total RNA was extracted from the frozen tomato plant tissues using a NucleoSpin® RNA plant kit (Macherey-Nagel, http://www.macherey-nagel.com) and was treated with RNasefree DNase I (Fermentas, http://www.fermentas.com). The total DNA-free RNA (400 ng) was used as a template for cDNA synthesis (iScript™ cDNA synthesis kit, Bio-Rad, http://www.bio-rad.com).

For real-time quantitative PCR, 5 μL of 25-fold diluted cDNA were used in a 25 μL PCR reaction containing 400 nM of each primer and 12.5 μL iQ™ SYBR Green Supermix (Bio-Rad). The PCR reactions were performed in a 96-well iCycler (Bio-Rad), with a temperature programme starting with 3 min at 95° C., then 40 cycles of 15 sec at 95° C. and 45 sec at 60° C. At the end, the melting temperature of the product was determined to verify the specificity of the amplified fragment.

The primers used for real-time quantitative PCR were designed with a computer program (Beacon Designer 5.01, Premier Biosoft International, http://www.premierbiosoft.com) as follows:

Slactin Forward primer:
(SEQ ID NO: 5)
5′-GGACTCTGGTGATGGTGTTAG-3′
Slactin Reverse primer:
(SEQ ID NO: 6)
5′-CCGTTCAGCAGTAGTGGTG-3′
SlARF9 Forward primer:
(SEQ ID NO: 7)
5′-CGTAGGCGTCAACAAATACTTAGAGG-3′
SlARF9 Reverse primer:
(SEQ ID NO: 8)
5′-TCCACTGTGAAGAAAGATCATCAATTCC-3′

The SlARF9 primer pair amplifies a 146 nucleotide fragment of the SlARF9 mRNA transcript (nucleotides 834-979 of SEQ ID NO: 1).

### 1.1.3 Isolation of the Genomic SlARF9 Sequences

For the structural characterization of SlARF9, several PCR products were amplified on genomic tomato DNA, isolated from young leaf tissue. The primers were derived from the coding sequence of SlARF9 (Genbank accession number BT013639). The PCR-products were completely sequenced and aligned to provide the information on the exon-intron structure of SlARF9. The genomic sequence is shown in SEQ ID NO: 3. The genomic sequence from cv Heinz 1706 (SEQ ID NO: 4) was derived from the SGN database, scaffold SL1.03sc03144 (http:solgenomics.net).

Genome walking (Genome Walker universal kit, BD Biosciences, http://www.bdbiosciences.com) on the SnaI (Fermentas) Genome Walker library using the gene-specific primer 5′-TTCTTCAGCCAGGAAATGACTATTGATAACTCG-3′ (reverse), and nested primer 5′-GGAGAATTCATATTCGGCTGAGAC-3′ (reverse) resulted in the isolation of a 3 kb fragment comprising the SlARF9 promoter (shown in SEQ ID NO: 3, upstream of the ATG codon). The Erase-a-Base system (Promega, http://www.promega.com) was used to generate subclones containing progressive unidirectional deletions of this fragment. Subsequently, these subclones were sequenced and aligned, using ClustalW (http://www.ebi.ac.uk/clustalW).

### 1.1.4 Plant Transformation

To generate SlARF9 over-expression lines (OE), the coding sequence of SlARF9 (forward 5′-CACCATGGCAACTATAAATGGGTGGTG-3′ (SEQ ID NO: 9), reverse 5′-TTAACTGTCTGCGCGAGACAGGG-3′- SEQ ID NO: 10) was cloned into the pENTR™/D-TOPO entry vector (Invitrogen). This clone was recombined with the pGD625 binary vector (Dr S. de Folter, Wageningen University, the Netherlands), in which the cauliflower mosaic virus (CaMV) 35S promoter was replaced for the ovary and young fruit specific TPRP-F1 promoter (Carmi et al., 2003, supra) by M. Busscher (Plant Research International, the Netherlands).

For the generation of the RNAi SlARF9 lines, a cDNA fragment of the SlARF9 middle region (amino acids 367-506, forward 5′-AAAAAGCAGGCTGTCCCACCAACCGCAGAGAAGAAC-3′- SEQ ID NO: 11; reverse 5′-AGAAAAGCTGGGTGCTGTAGTCGTGCCTCAGTAGTGC-3′- SEQ ID NO: 12) was cloned into the pDONR™221 entry vector (Invitrogen), which was subsequently recombined with the binary vector pK7GWIWG2(I) (Karimi et al., 2002, Trends in Plant Sciences 7, 193-195) in both sense and antisense orientation under the transcriptional regulation of the CaMV 35S promoter and terminator.

To generate the pSlARF9::GUS lines, the promoter fragment of SlARF9 (2200 bp, forward 5′-CACCTTTTCAAAGAGGTGTGACATTTTCAATAAC-3′- SEQ ID NO: 13; reverse 5′-CAACCTTCAATTCCAAAAACTAAAGAACACCC-3′- SEQ ID NO: 14) was cloned into the pENTR™/D-TOPO entry vector. This entry clone was recombined with the destination vector pKGWFS7 (Karimi et al., 2002, supra).

The transgenic tomato plants were generated by Agrobacterium tumefaciens mediated transformation, as described in de Jong et al. (2009, supra). Although grown on kanamycin-containing medium, possible escapes were detected by PCR with primers specific for the kanamycin resistance gene (forward 5′-GACTGGGCACAACAGACAATCG-3′, reverse 5′-GCTCAGAAGAACTCGTCAAGAAGG-3′) on genomic DNA.

Subsequently, lines were tested for tetraploidy, as only diploid lines were used for further analysis.

### 1.1.5 Histochemical Analysis of GUS Activity

Tissues of first generation adult plants (T1) and 15 d old seedlings (T2) of the pSlARF9::GUS lines were submerged in GUS-staining buffer containing 0.1% Triton X-100, 0.5 mM Fe2+CN, 0.5 mM Fe3+CN, 10 mM EDTA, 1 mg ml-1 X-Gluc, 0.1 mg ml-1 in 50 mM phosphate buffer, pH 7.0. After incubation at 37° C., the tissues were cleared with 70% ethanol and viewed under a stereomicroscope (Leica MZFL III, Leica Microsystems, http://leica-microsystems.com). For detailed analysis of lateral roots and ovules by light microscopy, the GUS-stained tissues were embedded in Technovit 7100 (Heraeus Kulzer, http://www.heraeus-kulzer.com). The embedded tissues were sliced into sections of 5 μm. The sections of the lateral roots were counterstained with 0.5% safranine, and subsequently partly de-stained with 70% ethanol. The sections were viewed under a Leitz Orthoplan microscope (Leica Microsystems). Images were made with a Leica digital camera (model DFC 420C; Leica Microsystems).

### 1.1.6 Quantification Methodology of Cell Area and Number of Cell Layers

Pericarp tissues of 7-8 mm diameter fruits were fixed in a 2% glutaraldehyde, 0.1 M phosphate buffer pH 7.2 solution for overnight at 4° C. Subsequently, the tissues were dehydrated in an ethanol series and embedded in Spurr. Sections of 1 μm were stained with a toluidine blue solution (0.1% in 1% borax).

Pericarp tissue of mature fruits at the breaker stage, were fixed in FAA (5% acetic acid, 3.7-4.1% formaldehyde solution and 50% ethanol), dehydrated in an ethanol series and subsequently embedded in Technovit. Sections of 5 μm were stained with a toluidine blue solution. The sections were viewed under a Leitz Orthoplan microscope (Leica Microsystems), and micrographs were made with a Leica digital camera (model DFC 420C; Leica Microsystems). These micrographs were used for further analysis.

For analysis of the 7-8 mm fruits, square sections of 0.16 mm² were delimited and positioned approximately 0.1 mm from the inner pericarp, including the epidermal layer. For analysis of the mature fruits, sections of 9 mm² were delimited and positioned approximately 1 mm from the inner pericarp. Then the total number of cells inside these squares was counted. Cells that were positioned in the sections for ⅔ of their size or more were included. For estimations on the number of cell layers within the pericarp, a line was drawn across the pericarp sections, and the number of cells along this line, including cell layers of the epidermis, exocarp, mesocarp and endocarp were scored. In total, 1 region per fruit and 5 fruits per line were analysed.

### 1.2. Results

### 1.2.1 Expression of SlARF9 in Tomato

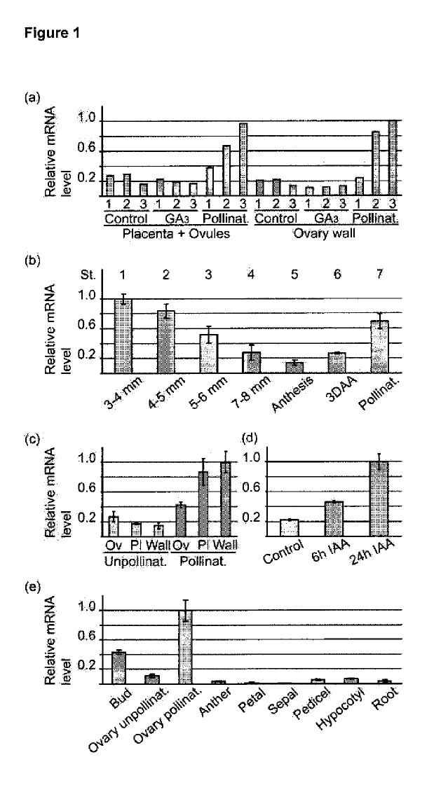

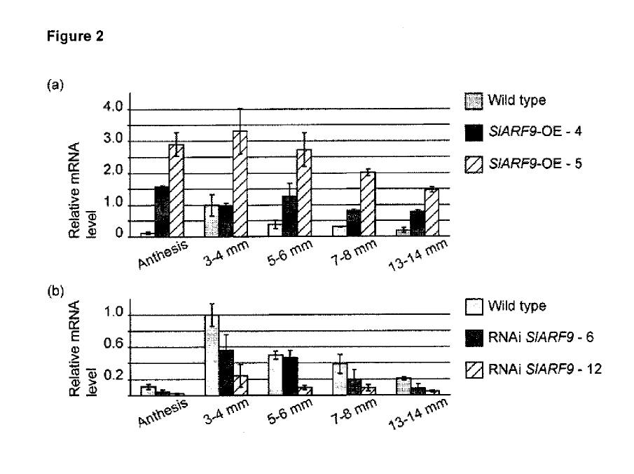

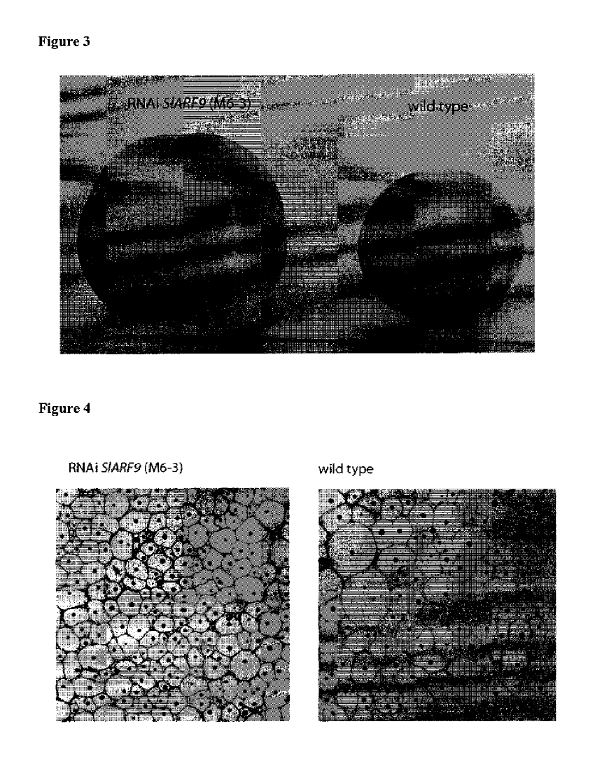

The relative transcript levels of SlARF9 increased within 2 days after pollination, but not after treatment with the plant hormone Gibberellic Acid (GA3). SlARF9 was expressed in the placental and ovular tissues as well as the ovary wall. See real-time quantitative PCR of FIG. 1a.

Analysis of ovary mRNA collected at various stages of flower development showed that the SlARF9 transcript was also highly abundant in the early stages of flower development, but decreased during the later stages, reaching the lowest level at anthesis (FIG. 1b). SlARF9 transcript level remained low, unless successful pollination and fertilization occurred. These processes increased the SlARF9 transcript levels mainly in the placental tissue and in the ovary wall (FIG. 1c).

Although GA treatment of unpollinated mature ovaries had no effect, auxin (IAA) application induced the expression of SlARF9 (FIG. 1d), suggesting that SlARF9 itself is responsive to auxin. So far, only the gene expressions of AtARF4, AtARF19 and Oryza sativa ARF23 were found to be auxin responsive (Ulmasov et al., 1999, Proceedings of the National Academy of Sciences, USA 96, 5844-5849; Okushima et al., 2005, The Plant Cell 17, 444-463; Overvoorde et al., 2005, The Plant Cell 17, 3282-3300; Wang et al., 2007, Gene 394, 13-24).

In other plant tissues, the levels of SlARF9 transcript were very low (FIG. 1e), suggesting that the SlARF9 function might be predominantly fruit specific.

To investigate the expression of SlARF9 in more detail, a SlARF9 promoter-GUS fusion was constructed, using the 2200 bp 5′ flanking sequence of the SlARF9 coding region ligated in front of the β-glucuronidase (GUS) coding sequence. Subsequently, this pSlARF9::GUS construct was introduced in tomato by Agrobacterium-mediated gene transfer. In 7 out of the 14 independent lines that were generated, GUS expression was observed in several tissues that were analysed after histochemical GUS staining.

In tomato fruits of 5-6 mm diameter, corresponding to approximately 8 DAP (Days after pollination), GUS staining was observed in the pericarp, in the outer cell layers of the placenta which develop into a gel-like substance, and in the ovules (data not shown).

Microscopic analysis of cross-sections through the ovules showed that the GUS staining is located at the micropylar end of the embryo sac. The area and location of the staining, suggest that GUS was not expressed by the embryo proper, which is at the 4-16 cell stage of development (Al-Hammadi et al., 2003, Plant Physiology 133, 113-125), but by the suspensor or by the wall ingrowths that developed quickly around its base (Briggs, 1995, Annals of Botany 76, 429-439). GUS was also expressed in the glandular hairs at the surface of leaf and stem, and in the axillary meristems, located in the shoot at the base of the leaves. Furthermore, GUS staining was observed in the primary root tips, early lateral root primordia and outgrowing lateral roots. The staining was located in the meristematic zone of the root tips, in the pericycle, and in a few cell layers of parenchyma.

Altogether, these results show that the activity of the SlARF9 promoter is not restricted to the fruit. Interestingly, most of the tissues in which this gene is transcribed are those in which many cell divisions occur.

### 1.2.2 Over-Expression and Silencing of SlARF9 in Tomato

To explore the physiological role of SlARF9 in tomato fruit set and development, transgenic tomato lines were generated in which the gene was over-expressed. For the production of these SlARF9 over-expression lines (SlARF9-OE), the coding sequence of SlARF9 was ligated to TPRP-F1 promoter, specific for the ovary and young fruit (Carmi et al., 2003, supra). From the 11 independent transgenic lines that were generated, the two SlARF9-OE lines with the highest expression, -4 and -5, respectively, were selected for further analysis.

In addition, transgenic tomato lines were generated in which the SlARF9 gene was silenced by RNA interference (RNAi) approach, using a 420 bp fragment based on the middle region of SlARF9 (amino acids 367-506). The specificity of this fragment was tested by genomic DNA Southern blot analysis, which resulted in one strong hybridization signal (data not shown).

The fragment was cloned into an RNAi binary vector, under the transcriptional regulation of the CaMV 35S promoter, and transferred to tomato by Agrobacterium-mediated transformation. In two out of the 12 transgenic lines that were generated, SlARF9 transcript levels were reduced. These two RNAi SlARF9 lines, -6, and -12, were used for further analysis.

Expression analysis of SlARF9 during several stages of early fruit development showed that in wild type, the relative mRNA level of SlARF9 rapidly increased after pollination and fertilization, and was highest in fruits of 3-4 mm in diameter, corresponding to 6 DAP. In the subsequent stages, transcript levels decreased again (FIG. 2).

In the SlARF9-OE lines, SlARF9 transcript levels were already high at anthesis, independently of pollination, and remained high for a longer period of time as compared to transcript levels in wild-type fruits (FIG. 2a).

In the RNAi SlARF9 lines, the expression pattern of SlARF9 was similar to that in wild type, but the overall transcript level was reduced with 40-70% (FIG. 2b).

Although the RNAi construct was under the regulation of the constitutive 35S promoter, no vegetative phenotypes, for example in root development or shoot branching, were observed. Nevertheless, both SlARF9-OE and RNAi SlARF9 lines showed a clear phenotype in fruit development. Histological cross-sections of fruits that were 7-8 mm in diameter were studied. These fruits were collected approximately 10 DAP, at the end of the cell division phase. Both number of cells per surface unit and number of cell layers in the pericarp were quantified. In general, the pericarp is differentiated in three layers: the endocarp, mesocarp and exocarp (Gillaspy et al., 1993, supra). In the pericarp of SlARF9-OE fruits the number of mesocarp cells per mm2 appeared to be significantly lower (P<0.05, Student's t test) as compared to the number of mesocarp cells in wild-type fruits, while the number of cells per mm² in the pericarp of the RNAi SlARF9 lines was significantly higher (P<0.05, Student's t test) (Table 1). Furthermore, the number of cell layers in the pericarp of the transgenic fruits seemed to be affected. In the SlARF9-OE fruits, this number was lower than in wild-type fruits, while in the RNAi SlARF9 fruits this number was increased. However, due to the great variation among the fruits, these differences were not statistically significant for all the transgenic lines (Table 1).

TABLE 1
Quantification of number of cells per surface unit or number of cell layers in the pericarp of
wild-type and transgenic fruits, 7-8 mm in diameter (10 DAP). The data represent the means ±
standard error of five fruits. For all measurements the differences between wild-type and
transgenic lines were tested for statistical significance. P-values (Student's t test) are indicated.

| | Cells/mm2 | | | Number of cell layers | | |
|---|---|---|---|---|---|---|
| | | | Percentage | | | Percentage |
| Line | | | of WT | | | of WT |
| Wild type | 781 ± 50 | | 100% | 29 ± 1 | | 100% |
| (control) | | | | | | |
| SlARF9-OE-4 | 452 ± 70 | (P < 0.05) | 58% | 27 ± 2 | (P = 0.41) | 93% |
| SlARF9-OE-5 | 353 ± 88 | (P < 0.05) | 54% | 22 ± 2 | (P < 0.05) | 76% |
| RNAi SlARF9-6 | 1246 ± 54 | (P < 0.05) | 160% | 35 ± 1 | (P < 0.05) | 121% |
| RNAi SlARF9-12 | 1256 ± 151 | (P < 0.05) | 161% | 32 ± 1 | (P = 0.13) | 110% |

In all, these findings suggest that with the over-expression of SlARF9, the total number of cells in the pericarp was decreased, while with the reduction of SlARF9 transcript levels by RNAi approach, the total number of cells in the pericarp was increased.

Analysis of fruit weight and diameter of mature fruit at breaker stage showed that fruits of SlARF9-OE lines were significantly smaller (weight P<0.05, diameter P<0.05, Student's t test) than wild-type fruits. In contrast, fruits of the RNAi SlARF9 lines were significantly bigger (weight P<0.05, diameter P<0.05) than wild-type fruits (Table 2).

TABLE 2
Analysis of fruit weight, and size (diameter) of mature
wild-type and transgenic fruits, collected at breaker stage.
The data represent the means ± standard error of 5-20 fruits.
For all measurements, the differences between
wild-type and transgenic lines were statistically
significant (P < 0.05, Student's t test).

| | Weight (g) | | Diameter (mm) | |
|---|---|---|---|---|
| Line | | % of WT | | % of WT |
| Wild type | 77 ± 3.0 | 100% | 54 ± 0.9 | 100% |
| (control) | | | | |
| SlARF9-OE-4 | 53 ± 3.4 | 67% | 48 ± 1.1 | 89% |
| SlARF9-OE-5 | 55 ± 3.6 | 71% | 48 ± 1.2 | 89% |
| RNAi SlARF9-6 | 119 ± 13.3 | 154% | 63 ± 2.5 | 117% |
| RNAi SlARF9-12 | 102 ± 7.2 | 132% | 59 ± 1.4 | 109% |

Moreover, microscopic analysis showed that the number of cell layers in the pericarp of RNAi SlARF9 fruits and the number of cells per surface unit was higher than in wild-type fruits (Table 3). The greater fruit size of the RNAi SlARF9 fruits (see FIG. 3) is probably mainly caused by extra anticlinal cell divisions in the pericarp.

The micrographs also showed that the pericarp cells of RNAi SlARF9 fruits were not only more, but also smaller than in wild type fruits (see FIG. 4). In Table 3 below cell size (cell surface) was calculated from Cells/mm².

TABLE 3
Quantification of number of cells per surface unit or number of cell
layers in the pericarp of mature wild-type and RNAi SlARF9 fruits, collected
at breaker stage. The data represent the means ± standard error
of five fruits. The p-values (P; Student's t tests) are indicated.

| | | % of wild | Cell surface | % of wild | Number of | % of wild |
|---|---|---|---|---|---|---|
| Line | Cells/mm² | type (WT) | (mm²) | type (WT) | cell layers | type (WT) |
| Wild type | 6.88 ± 0.51 | 100% | 0.15 ± 0.01 | 100% | 27 ± 2 | 100% |
| RNAi | 7.44 ± 0.55 | 108% | 0.13 ± 0.01 | 87% | 32 ± 3 | 118% |
| SlARF9-6 | (P = 0.48) | | | | (P = 0.20) | |
| RNAi | 9.60 ± 1.19 | 139% | 0.10 ± 0.01 | 67% | 33 ± 2 | 122% |
| SlARF9-12 | (P = 0.08) | | | | (P = 0.05) | |

### Discussion

When the ovary transforms into a fruit after pollination and fertilization, several genes involved in cell cycle and cell expansion are induced (Vriezen et al., 2008, supra; Pascual et al., 2009, BMC Plant Biology 9, 67; Wang et al., 2009, The Plant Cell 21, 1428-1452). The induction of these genes is likely to be mediated by the hormones auxin and gibberellin, since treatments of unpollinated ovaries with auxin resulted in the formation of fruits with a higher number of pericarp cells, whereas the pericarp of GA-induced fruits contained fewer but bigger cells (Bünger-Kibler and Bangerth, 1982, supra; Serrani et al., 2007, supra). In accordance, our previous work to identify genes involved in fruit set showed that the expression of both auxin- and GA-related genes were up-regulated after pollination (Vriezen et al., 2008, supra).

Here we describe the functional analysis of a gene designated SlARF9. In Arabidopsis, AtARF9 has been characterized as a transcriptional repressor (Ulmasov et al., 1999 supra; Tiwari et al., 2003, supra), but the function of this transcription factor is still largely unknown as most T-DNA insertion mutant lines did not show an obvious phenotype (Okushima et al., 2005, supra). However, AtARF9 mutant lines lacking the 3′-end of the transcript over-responded after gravistimulation, suggesting that AtARF9 might be involved in the gravitropic signal transduction. Furthermore, AtARF9 was found to be expressed in the suspensor of the embryo, and double knock-out lines, in which both ARF9 and ARF13 were silenced, showed that AtARF9 is necessary for the control of suspensor development (Liu et al., 2008, supra).

AtARF9 function has not been related to Arabidopsis fruit development. The only ARF known to be involved in this process is FRUIT WITHOUT FERTILIZATION (FWF)/ARF8, as fwf/arf8 mutant lines formed parthenocarpic siliques (Goetz et al., 2006, supra). In tomato, transgenic lines with reduced SlARF7 transcript levels also formed parthenocarpic fruits, which indicate that SlARF7 acts as a negative regulator of fruit set (de Jong et al., 2009, supra). The only other member of the tomato ARF family characterized so far is the DEVELOPMENTALLY REGULATED GENE 12 (DR12), the homologue of AtARF4. The mRNA levels of DR12 increased throughout fruit development, and reached the highest level at the early red-stage fruit. Down-regulation of this gene by anti-sense approach affected the fruit firmness at the red stage (Jones et al., 2002, supra).

In contrast, SlARF9 was found to be mainly expressed at the early stages of fruit development. These stages correspond to the period in which tomato fruit growth mainly depends on cell division (Mapelli et al., 1978, supra; Bünger-Kibler and Bangerth, 1982, supra; Gillaspy et al., 1993, supra). SlARF9 expression was also induced in unpollinated ovaries treated with auxin, while SlARF9 transcript levels did not increase in parthenocarpic fruits formed after gibberellin application.

Furthermore, the results of the GUS analysis showed that SlARF9 was also expressed in other plant tissues in which many cell divisions occur. In all, these findings suggest that SlARF9 regulates cell division activity.

Despite the fact that SlARF9 cannot be considered as a fruit specific gene, the RNAi SlARF9 lines only displayed a fruit phenotype, indicating that in other plant tissues SlARF9 may act redundantly with other members of the ARF protein family.

Decreased SlARF9 transcript levels resulted in the formation of significantly bigger fruits probably mainly due to extra anticlinal cell divisions in the pericarp, whereas increased SlARF9 transcript levels led to the formation of smaller fruits as compared to wild type. These opposite indicate that SlARF9 regulates cell division, i.e. the SlARF9 protein is a negative regulator (repressor) of cell division.

The finding that downregulation of SlARF9 leads to significantly bigger fruit compared to wild type plants makes this gene useful for modifying fruit size in plants, either transgenically or through the provision of (non-transgenic) plants comprising mutant slarf9 alleles or mutations in the SlARF9 promoter whereby SlARF9 transcript levels and protein levels are reduced or abolished.

### Example 2

### SlARF9 Promoter Analysis in Arabidopsis

### 2.1 Material and Methods

### 2.1.1 Plant Materials and Growth Conditions

The Arabidopsis thaliana transgenic plants in Col-0 background were grown under standardized greenhouse conditions, with a temperature of 22° C. and a 16 h light/8 h dark cycle. Seeds that resulted from floral dip transformation were sterilized by treatment with 100% ethanol for 1 min and with a 2% hypochloride solution for 10 min. After rinsing three times with sterile distilled water, seeds were sown on ½ Murashige and Skoog (MS) culture medium, including Gamborg B5 vitamins, 0.05% (w/v) MES, 0.7% (w/v) phytoagar and 30 mg L-1 kanamycin, pH 5.7. After 10 d incubation in a growth chamber (16 light/8 h dark, 22° C.), resistant plants were transferred to soil.

Tomato plants (Solanum lycopersicum cv. Moneymaker) were grown as previously described in de Jong et al. (2009, supra). To analyse the expression of the auxin response genes, ovaries of emasculated flowers were treated with 2 μL of 1 mM 4-C1-IAA (Sigma-Aldrich, http://www.sigmaaldrich.com) in 2% ethanol. The treatment was repeated 6 h after the first application. Control flowers were collected at the stage of anthesis. The collected tissues were frozen in N2 and stored at −80° C. until RNA extraction.

2.1.2 Plant transformation

To generate transgenic promoter::uidA lines, promoter fragments of SlARF9 (2200 bp, forward 5′-CACCTTTTCAAAGAGGTGTGACATTTTCAATAAC-3′- SEQ ID NO: 13; reverse 5′-CAACCTTCAATTCCAAAAACTAAAGAACACCC-3′- SEQ ID NO: 14) and AtARF9 (2466 bp, forward 5′-AAAAAGCAGGCTTGGTGGTGGGTTTTAAGGCATC-3′; reverse 5′-AGAAAAGCTGGGTCACACAGTCTCTCTATCTCTCTCC-3′) were cloned into the pENTR™/D-TOPO or pDONR™221 entry vector (Invitrogen, http://www.invitrogen.com). Subsequently, the entry clones were recombined with the destination vector pKGWFS7 (Karimi et al., 2002, Trends in Plant Sciences 7, 193-195). These constructs were transformed into Agrobacterium tumefaciens strain EHA105 using freeze-thaw transformation (Chen et al., 1994, Biotechniques 16, 664-670). The transformation of Arabidopsis plants was performed using the floral dip method as described by Clough and Bent (1998, The Plant Journal 16, 735-743).

### 2.1.3 Hormone Response Assay

To test the auxin responsiveness of the SlARF9 and AtARF9 promoters, 10-d-old seedlings and mature leaves of the Arabidopsis pSlARF9::GUS lines were incubated in 0.05% ethanol or 5004 indole-acetic-acid (IAA) in 0.05% ethanol. After 3 h and 9 h, the tissues were frozen in N2 and stored at −80° C. until RNA extraction.

### 2.1.4 Real-Time Quantitative PCR

The total RNA was isolated and reverse transcribed to cDNA, following the protocol described in Example 1.1.2. Also for the real-time quantitative PCR, the same conditions were used as in Example 1.1.2. The sequences of the primers used for real-time quantitative PCR were SEQ ID NO 7 and 8 for AlARF9 and the following for AtARF9:

Forward 5′-AGAAGCCATGAGCAATAAGTTCTCTGTAGG-3′
Reverse R 5′-GGGAGCAGTCTTTCACACCAATAACC-3′

And for GUS (uidA):

Forward 5′-CTCCTACCGTACCTCGCATTAC-3′
Reverse 5′-CCGTTGACTGCCTCTTCGC-3′

### 2.1.5 Histochemical Analysis of GUS Activity

Tissues of adult plants (T1) and 10-d-old seedlings (T2) of the Arabidopsis pSlARF9::GUS and pAtARF9::GUS lines were submerged in GUS staining buffer containing 0.1% Triton X-100, 0.5 mM Fe2+CN, 0.5 mM Fe3+CN, 10 mM EDTA, 1 mg mL-1 X-Gluc, 0.1 mg mL-1 in 50 mM phosphate buffer, pH 7.0. After incubation at 37° C., the tissues were cleared in 70% ethanol. The stained tissues were viewed under a stereomicroscope (Leica MZFL III, http://leica-microsystems.com). Images were made with a Leica digital camera (model DFC 420C; Leica Microsystems).

### 2.1.6 Promoter Analysis in Silico

The promoter sequences of SlARF9 and AtARF9 (At4g2323980) including the 5′ untranslated regions of these genes were analyze using PlantCARE (Lescot et al., 2002 Nucleic Acids Research 30, 325-327 http://bioinformatics.psb.ugent.be/webtools/plantcare/html) and PLACE (Higo et al., 1999, Nucleic Acids Research 27, 297-300, http://www.dna.affrc.go.jp/PLACE/).

### 2.2 Results

### 2.2.1 In Silico Promoter Analysis of SlARF9 and AtARF9

The transcript levels of Solanum lycopersicum ARF9 (SlARF9) increased within 48 h after pollination. However, the expression of SlARF9 was also induced in unpollinated ovaries treated with the hormone auxin (FIG. 1c). So far, only the gene expressions of AtARF4, AtARF19 and Oryza sativa ARF23 were found to be auxin induced (Ulmasov et al., 1999, supra; Okushima et al., 2005, supra; Overvoorde et al., 2005, supra; Wang et al., 2007, supra). In this study, we analysed the 1500 bp 5′ region upstream from the SlARF9 gene with the PlantCARE (Lescot et al., 2002, supra) and PLACE (Higo et al., 1999, supra) software for the presence of auxin-related cis-acting regulatory elements, which resulted in the identification of two degenerated Auxin Response Elements (AuxREs). These elements are typically found in the promoter sequences of auxin response genes and are bound by the ARF transcription factors (Ulmasov et al., 1999, supra). Furthermore, the promoter sequence contained several NTBBF1ARROLB-elements. These elements were first identified in the promoter sequence of rolB, one of the oncogenes present in the T-DNA sequence of Agrobacterium rhizogenes, and are involved in the auxin-inducible expression of rolB in plants (Baumann et al., 1999, The Plant Cell 11, 323-333). Both AuxRE's and NTBBF1ARROLB-elements were also overrepresented in the promoter sequences of SlIAA2 and SlIAA14. These genes are two members of the tomato Aux/IAA gene family, a family of transcriptional repressors that regulate the expression of auxin-responsive genes. However, many Aux/IAAs are induced by auxin themselves (Reed, 2001, Trends in Plant Science 6, 420-425). SlIAA2 and SlIAA14 expression was up-regulated after pollination (Vriezen et al., 2008, supra), but also in unpollinated ovaries treated with auxin, similar to SlARF9 (FIG. 1). Analysis of the AtARF9 promoter sequence resulted in the identification of several auxin-related cis-acting elements (results not shown). AuxREs, degenerate AuxREs, and NTBBF1ARROLB-elements were present. Furthermore, the element ASF1MOTIFCAMV was overrepresented. This element was found in a number of auxin-responsive genes, and was originally detected in the CaMV 35S promoter (Liu and Lam, 1994, The Journal of Biological Chemistry 269, 668-675). Similar auxin-related elements were present in the promoter sequences of the auxin-inducible AtIAA1 and AtIAA5 (Abel et al., 1995, Journal of Molecular Biology 251, 533-549).

The in silico promoter analysis showed, that the 5′-end upstream regions of the ARF9 genes hold the same auxin-related cis-acting elements as found in the promoter regions of the auxin-inducible Aux/IAA genes, suggesting that the expression of both SlARF9 and AtARF9 is regulated by auxin. Moreover, most of the identified cis-elements were similar in the tomato and Arabidopsis promoter sequences.

### 2.2.2 Auxin-Inducible Expression of SlARF9 and AtARF9

As similar auxin-related cis-acting elements were detected in the promoter sequences of SlARF9 and AtARF9, one would expect that the auxin-inducibility of the SlARF9 promoter is maintained in Arabidopsis. Therefore, the 2200 bp 5′-end flanking sequence of the SlARF9 coding region was ligated in front of the β-glucuronidase (GUS) coding sequence of the uidA gene. Subsequently, this pSlARF9::uidA construct was introduced in Arabidopsis by Agrobacterium-mediated gene transfer. Mature rosette leafs of the generated transgenic lines were mock-treated or treated in a solution of 50 μM indole-acetic-acid (IAA). After 3 h and 9 h of incubation, the leaf samples were analysed for uidA-expression by real-time quantitative PCR. Furthermore, these tissue samples were used to study the auxin-inducibility of AtARF9 expression.

Although the expression of uidA could be detected, the levels were too low to be quantified reliably. In contrast, AtARF9 transcript levels could be quantified. The transcript levels of AtARF9 increased 3 h and 9 h after IAA-treatment. However, expression was also up-regulated in the mock-treated samples (data not shown). Therefore, the expressions of AtIAA1 and AtIAA5 were analysed. The transcript levels of these genes were strongly induced in the IAA-treated samples, while the transcript levels remained low in the mock-treated samples (data not shown). These results showed that the experimental set-up was correct. The same experiment was repeated on 10-d-old seedlings of the pSlARF9::uidA lines, but similar results were obtained. The findings indicate that despite the putative auxin-related elements that were present in the promoter sequence, expression of AtARF9 is not induced by auxin.

### 2.2.3 Expression Patterns of SlARF9 and AtARF9 in Arabidopsis

Since the expression of the pSlARF9::uidA lines was too low to be quantified, the question raised whether the regulatory elements present in the promoter sequence of SlARF9 were still functional in Arabidopsis. Therefore, the pSlARF9::uidA lines were analysed after histochemical GUS staining In 10-d-old seedlings the stipules, young developing leaves, trichomes of the developing leaves, the early lateral root primordia, and the root tips of the lateral roots stained blue. Furthermore, GUS activity was detected in several tissues during flower morphogenesis. The youngest flower buds displayed no GUS activity, but in larger buds GUS-expression was observed in the stigma and the tip of the sepals. After pollination, GUS activity was also detected in the developing seeds. However, in the siliques collected approximately 6 d after pollination (DAP), no GUS activity was observed.

To investigate the expression of AtARF9 in more detail, transgenic lines were generated, using the 2466 bp 5′-end flanking sequence of the AtARF9 coding region ligated in front of the uidA coding sequence, and analysed after histochemical GUS staining. In 10-d-old seedlings, the stipules and trichomes of the developing leaves were stained. Moreover, GUS staining could be detected in the central cylinder of the roots. During flower morphogenesis, no GUS expression was observed in the youngest buds. In the larger buds only the stamens were stained. A closer view showed that this staining was located in the developing pollen grains, the tapetum cells and the parenchym cells of the anthers. In mature flower buds collected just before anthesis, the GUS expression in the stamen was decreased, but increased throughout the gynoecium. After pollination, the GUS expression of the gynoecium became more apparent, and was maintained in the developing silique. Furthermore, the silique abscission zone was stained.

These results demonstrated that the regulatory elements present in the promoter sequence of SlARF9 were still functional in Arabidopsis. However, the SlARF9 promoter and AtARF9 promoter are active in different tissues.

### Example 3

### slarf9 TILLING Mutants

### 3.1 Tomato TILLING Population

A highly homozygous inbred line used in commercial processing tomato breeding was used for mutagenesis treatment with the following protocol. After seed germination on damp Whatman® paper for 24 h, ˜20,000 seeds, divided in 8 batches of 2500 respectively, were soaked in 100 ml of ultra pure water and ethyl methanesulfonate (EMS) at a concentration of 1% in conical flasks. The flasks were gently shaken for 16 h at room temperature. Finally, EMS was rinsed out under flowing water. Following EMS treatment, seeds were directly sown in the greenhouse. Out of the 60% of the seeds that germinated, 10600 plantlets were transplanted in the field. From the 8810 M1 lines that gave fruits, two fruits per plant were harvested. DNA was isolated from seeds coming from the first fruit, constituting the M2 population DNA stock. These were selfed and M3 seeds were isolated from the fruits and the seeds were used for DNA isolation and constitute the M3 population DNA bank.

3.2 Target SlARF9 Gene for PCR Amplification from TILLING Population

DNA of the tomato TILLING population described above was screened for single nucleotide polymorphisms in the SlARF9 target gene. For this purpose the following PCR primer pairs were designed to amplify conserved parts of the N-terminal B3 superfamily DNA binding domain (DBD). Mutations in this region can lead to substitution of conserved residues which can lead to a decreased affinity of the mutated SlARF9 protein for promoter sequences of its target genes. In addition, introduction of a potential stop codon in this region will lead to very short truncated SLARF9 protein that will very likely to be inactive/non-functional.

Primers designed to screen the EMS mutagenized tomato
population for mutations in the SlARF9
DNA binding domain. F = Forward primer;
R = reverse primer. AA targeted
are the residues encoded by the amplification product.

| | | exon nr | AA |
|---|---|---|---|
| primer name | Primer sequence 5′-3′-end | targeted | targeted |
| F-4008 | AATTTGAGAATTTTGGAGCTTTTT | 2 | K20-Q56 |
| | (SEQ ID NO: 15) | | |
| R-4009 | AGAACTTAGCCTTGAATCAACCA | 2 | K20-Q56 |
| | (SEQ ID NO: 16) | | |
| F-4010 | TGAGTTTCAGGTAAAAAAGATGC | 2 | K20-Q56 |
| | (SEQ ID NO: 17) | | |
| R-4011 | ACAGAAAAAAACAACAACACAGAA | 2 | K20-Q56 |
| | (SEQ ID NO: 18) | | |
| F-4030 | CCCCTGTTTTGACAAGTTGTTG | 6 | D160-R187 |
| | (SEQ ID NO: 19) | | |
| R-4031 | CATTGATATCTTCTGTTACACTCTAC | 6 | D160-R187 |
| | (SEQ ID NO: 20) | | |
| F-4032 | GTAGAGTGTAACAGAAGATATCAATG | 7 | G188-R218 |
| | (SEQ ID NO: 21) | | |
| R-4033 | CCAGAGAGAGATACCTCAAGAATAC | 7 | G188-R218 |
| | (SEQ ID NO: 22) | | |

Other primer pairs are designed to screen less conserved parts of the DBD if necessary.

The primer pairs were used to amplify target sequences from the M2 or M3 DNA of the TILLING population and heteroduplexes between mutant and wild type target sequences were detected using CSCE or HRM as described below. The ID number of the DNA samples is linked to seed batches of plants carrying the wild type allele or the mutated allele either in heterozygous or in homozygous form.

Seeds were germinated and the presence of the particular mutation in individual plants is confirmed by PCR using primers flanking the mutated site and genomic DNA of these plants as templates. DNA sequencing of the fragments identifies mutants homozygous and heterozygous for the expected mutation. Homozygous mutants were selected or obtained after selfing and subsequent selection and the effect of the mutation on the corresponding protein and phenotype of the plant is determined.

The following mutants were identified using primer pair SEQ ID NO: 19 and 22 (mutant 1719, mutant 2484, mutant 6725 and mutant 6932) or primer pair SEQ ID NO: 15 and 16 (mutant 3175) and seeds were deposited at the NCIMB under the Accession numbers given below.

| | | | Mutation in |
|---|---|---|---|
| Mutant | NCIMB | Mutation in ARF9 | ARF9 protein |
| tomato | Accession | genomic DNA | (SEQ ID NO: |
| plant | Number | (SEQ ID NO: 3) | 2 or 3) |
| Mutant 1719 | NCIMB 41827 | Nucleotide 3529 | In intron between |
| | | is changed from c | exon 6 and 7, next |
| | | to a: cag → aag | to nucleotides ag, |
| | | | necessary for |
| | | | correct pre-mRNA |
| | | | splicing |
| Mutant 2484 | NCIMB 41828 | nucleotide 3540 is | amino acid 191 in |
| | | changed from c to t: | exon 7 is changed |
| | | cgg → tgg | from Arg to Trp |
| Mutant 3175 | NCIMB 41829 | Nucleotide 2254 is | amino acid 52 in |
| | | changed from g to | exon 2 is changed |
| | | a: ggc → agc | from Gly to Ser |
| Mutant 6725 | NCIMB 41830 | Nucleotide 3546 is | amino acid 193 in |
| | | changed from c to t: | exon 7 is changed |
| | | cat → tat | from His to Tyr |
| Mutant 6932 | NCIMB 41831 | Nucleotide 3546 is | amino acid 193 in |
| | | changed from c to t: | exon 7 is changed |
| | | cat → tat | from His to Tyr |

Thus, one slarf9 mutant may affect pre-mRNA splicing (mutant 1719), one mutant lies in exon 2 (mutant 3175) and three mutants lie in exon 7 (mutants 2484, 6725 and 6932), which is part of the b3-derived DNA binding domain of SlARF9.

Plants comprising mutations in the target sequence, such as the above mutant plants or plants derived therefrom (e.g. by selfing or crossing) and comprising the mutant slarf9 allele, are screened phenotypically for the development of significantly larger fruits.

Two mutant alleles can also be combined in one plant by crossing plants having different mutations, to determine the effect on fruit size.

### 3.3 Conformation Sensitive Capillary Electrophoresis (CSCE)

Multiplex PCR reactions are performed in 10 μl volume with 0.15 ng, 4 times pooled genomic DNA. Labeled primers are added to the PCR master mix to a concentration 5 times lower (1 μM) than that of the unlabeled primers. Post PCR, samples are diluted 10 times. Before the CSCE run, 2 μl of the diluted products are added to 38 μl of MQ water.

The samples are loaded on 50 cm capillaries (injection time and voltage: 16 seconds, 10 KVolts; Run voltage: 15 KVolts) from the ABI 3130x1 apparatus filled with semi-denaturating polymers of the following composition: 5 g Conformation Analysis Polymer (CAP) (Applied Biosystems, 434037, 9%), 2.16 g Ureum, 0.45 g 20×TTE (national diagnostics, EC-871), completed with MQ water up to 9 g. The running buffer is prepared with 1× diluted TTE and 10% glycerol. The oven temperature is set to 18° C.)

Raw data is analysed with the HeteroDuplex Analysis (HDA) software from BioNumerics, The program differentiates peak patterns of hetero-duplexes (mutant) and homo-duplex molecules (wild type) thus providing the possibility of selecting DNA-pools containing an individual line mutated in the target gene.

### 3.4 High Resolution Melt Curve Analysis (HRM)

The LCgreen PCRs are performed on 8× flat pools in FramStar 96-wells plates (4titude, UK). 2 μl (15 ng) of pooled DNA is mixed with 2 μl of F-524 Phire™ 5× reaction buffer (FINNZYMES, Finland), 0.1 μl Phire™ Hot Start DNA Polymerase (FINNZYMES, Finland), 1 μl LCGreen™ Plus+ (BioChem, USA), 0.25 μl of 5 mM primers, and completed to 10 μl with MQ water) according to manufacturer recommendations. Pools containing a mutation are screened using a LightScanner® System (Idaho Technology Inc., USA). Positive pools are selected by analyzing the melting temperature profiles; when the pool contains a mutation it will show a lower melting temperature.

### Example 4

### Transfer of Mutant Slarf9 Alleles into Tomato Cultivars

TILLING mutants comprising a mutant slarf9 allele, such as any one of the above mutant alleles, are crossed with different tomato lines in order to transfer the mutant allele into these lines, generating tomato plants with good agronomic characteristics and significantly larger fruits.

A TaqMan® SNP Genotyping Assays (Applied Biosystems) marker is developed to identify the presence of the modified nucleotide. This assay is used for Marker-assisted foreground selection which is effective for the transfer of recessive genes to a required background, for example commercial tomato parent lines, since their classical transfer requires additional recurrent selfing generations (Ribaut et al. Plant Molecular Biology Reporter 15:154-162).

1. A non-transgenic plant, fruit, seed, or part thereof comprising an Auxin Response Factor 9 (slarf9) allele in its genome, whereby the slarf9 allele encodes a S1ARF9 protein comprising at least 60% amino acid sequence identity to SEQ ID NO: 2 and, comprises one or more mutations in its nucleotide sequence and whereby the plant comprising said mutant allele in its genome produces significantly larger fruits compared to the plant comprising a wild type SlARF9 allele in its genome.

2. The plant according to claim 1, wherein said one or more mutations confer loss-of-function or reduced-function onto the encoded SlARF9 protein.

3. The plant according to claim 1, wherein said mutant slarf9 allele is present in homozygous form.

4. The plant according to claim 1, wherein said plant belongs to the family Solanaceae.

5. The plant according to claim 1, wherein average fruit size is at least 110% of the fruit size of the plant comprising the wild type SlARF9 allele.

6. The plant according to claim 1, wherein the plant is a hybrid plant.

7. The fruit, seed, or a part of a plant according to claim 1.

8. A method of generating transgenic or non-transgenic plants which produce large fruits comprising introducing into said plants a nucleic acid sequence encoding an SlARF9 protein, wherein the SlARF9 protein comprises at least 60% amino acid sequence identity to SEQ ID NO: 2.

9. A method of producing fruit with increased size comprising introducing into a fruit producing plant a nucleic acid sequence encoding a SlARF9 protein, wherein the S1ARF9 protein comprises at least 60% amino acid sequence identity to SEQ ID NO: 2.

10. A transgenic plant comprising integrated in its genome a chimeric gene, wherein said chimeric gene comprises a promoter active in plant cells operably linked to a nucleic acid sequence comprising a sense and/or antisense sequence of a SlARF9 gene which upon transcription silences endogenous SlARF9 gene expression and wherein said plant produces larger fruit compared to a plant lacking said chimeric gene.

11. The plant according to claim 10, wherein said endogenous SlARF9 gene encodes a protein comprising at least 60% amino acid sequence identity to SEQ ID NO: 2.

12. The plant according to claim 10, wherein said transcription regulatory sequence is a constitutive promoter, an inducible promoter, a tissue-specific promoter, or a developmentally regulated promoter.

13. The plant according to claim 10, wherein the plant is from a genus of: Solanum, Cucumis, Citrullus, Capsicum, Malus, Zea, Oryza, Triticum, Hordeum, Avena, or Sorghum.

14. A seed, a fruit, or part of a plant according to claim 10, wherein said seed, fruit, or part of a plant comprises a chimeric gene comprising a promoter active in plant cells operably linked to a nucleic acid sequence comprising a sense and/or antisense sequence of a SlARF9 gene which upon transcription silences endogenous SlARF9 gene expression.

15. A chimeric gene comprising a constitutive-, tissue specific-, inducible- or developmentally regulated promoter active in plant cells, operably linked to a nucleic acid sequence comprising a sense and/or antisense fragment of a SlARF9 gene which upon transcription silences endogenous SlARF9 gene, wherein said endogenous SlARF9 gene comprises at least 70% sequence identity to SEQ ID NO: 1 or SEQ ID NO:3 from nucleotide 2005 to 5879.

16. A non-transgenic tomato plant, or seeds, progeny or tomato fruits thereof, said plant being obtainable by tilling, comprising a mutant slarf9 allele in its genome, wherein said mutant allele encodes an SlARF9 protein having reduced-function or loss-of-function compared to the wild type SlARF9 protein.

17. The plant according to claim 4, wherein the plant belongs to the genus Solanum.

18. The plant according to claim 17, wherein the plant is the species Solanum lycopersicum.
